SEQUENCE LISTING (158 sequences)

SEQ ID NO: 1 - protein, 163 aa, Escherichia coli
MKLKAIILATGLINCIVFSAQAVDTTITVTGNVLQRTCNVPGNVDVSLGNLYVSDFPNAGSGSPWVNFDLSLTGCQNMNTVRATFSGTADGQTYYANTGNAGGIKIEIQDRDGSNASYHNGMFKTLNVQNNNATFNLKARAVSKGQVTPGNISSVITVTYTYA

SEQ ID NO: 2 - protein, 673 aa, Escherichia coli
MKMTRLYPLALGGLLLPAIANAQTSQQDESTLVVTASKQSSRSASANNVSSTVVSAPELSDAGVTASDKLPRVLPGLNIENSGNMLFSTISLRGVSSAQDFYNPAVTLYVDGVPQLSTNTIQALTDVQSVELLRGPQGTLYGKSAQGGIINIVTQQPDSTPRGYIEGGVSSRDSYRSKFNLSGPIQDGLLYGSVTLLRQVDDGDMINPATGSDDLGGTRASIGNVKLRLAPDDQPWEMGFAASRECTRATQDAYVGWNDIKGRKLSISDGSPDPYMRRCTDSQTLSGKYTTDDWVFNLISAWQQQHYSRTFPSGSLIVNMSQRWNQDVQELRAATLGDARTVDMVFGLYRQNTREKLNSAYDMPTMPYLSSTGYTTAETLAAYSDLTWHLTDRFDIGGGVRFSHDKSSTQYHGSMLGNPFGDQGKSNDDQVLGQLSAGYMLTDDWRVYTRVAQGYKPSGYNIVPTAGLDAKPFVAEKSINYELGTRYETADVTLQAATFYTHTKDMQLYSGPVGMQTLSNAGKADATGVELEAKWRFAPGWSWDINGNVIRSEFTNDSELYHGNRVPFVPRYGAGSSVNGVIDTRYGALMPRLAVNLVGPHYFDGDNQLRQGTYATLDSSLGWQATERMNISVYVDNLFDRRYRTYGYMNGSSAVAQVNMGRTVGINTRIDFF

SEQ ID NO: 3 - protein, 246 aa, Escherichia coli
MNKVFVVSVVAAACVFAVNAGAKEGKSGFYLTGKAGASVMSLSDQRFLSGDEEETSKYKGGDDHDTVFSGGIAVGYDFYPQFSIPVRTELEFYARGKADSKYNVDKDSWSGGYWRDDLKNEVSVNTLMLNAYYDFRNDSAFTPWVSAGIGYARIHQKTTGISTWDYEYGSSGRESLSRSGSADNFAWSLGAGVRYDVTPDIALDLSYRYLDAGDSSVSYKDEWGDKYKSEVDVKSHDIMLGMTYNF

SEQ ID NO: 4 - protein, 166 aa, Escherichia coli
MKLKAIILATGLINCIAFSAQAVDTTITVTGRVLPRTCTIGNGGNPNATVVLDNAYTSDLIAANSTSQWKNFSLTLTNCQNVNNVTSFGGTAENTNYYRNTGDATNIMVELQEQGNGNTPLKVGSTKVVTVSNGQATFNLKVRAVSKGNAGAGSINSQITVTYTYA

SEQ ID NO: 5 - protein, 1295 aa, Escherichia coli
MNKIYSLKYSAATGGLIAVSELAKRVSGKTNRKLVATMLSLAVAGTVNAANIDISNVWARDYLDLAQNKGIFQPGATDVTITLKNGDKFSFHNLSIPDFSGAAASGAATAIGGSYSVTVAHNKKNPQAAETQVYAQSSYRVVDRRNSNDFEIQRLNKFVVETVGATPAETNPTTYSDALERYGIVTSDGSKKIIGFRAGSGGTSFINGESKISTNSAYSHDLLSASLFEVTQWDSYGMMIYKNDKTFRNLEIFGDSGSGAYLYDNKLEKWVLVGTTHGIASVNGDQLTWITKYNDKLVSELKDTYSHKINLNGNNVTIKNTDITLHQNNADTTGTQEKITKDKDIVFTNGGDVLFKDNLDFGSGGIIFDEGHEYNINGQGFTFKGAGIDIGKESIVNWNALYSSDDVLHKIGPGTLNVQKKQGANIKIGEGNVILNEEGTFNNIYLASGNGKVILNKDNSLGNDQYAGIFFTKRGGTLDLNGHNQTFTRIAATDDGTTITNSDTTKEAVLAINNEDSYIYHGNINGNIKLTHNINSQDKKTNAKLILDGSVNTKNDVEVSNASLTMQGHATEHAIFRSSANHCSLVFLCGTDWVTVLKETESSYNKKFNSDYKSNNQQTSFDQPDWKTGVFKFDTLHLNNADFSISRNANVEGNISANKSAITIGDKNVYIDNLAGKNITNNGFDFKQTISTNLSIGETKFTGGITAHNSQIAIGDQAVVTLNGATFLDNTPISIDKGAKVIAQNSMFTTKGIDISGELTMMGIPEQNSKTVTPGLHYAADGFRLSGGNANFIARNMASVTGNIYADDAATITLGQPETETPTISSAYQAWAETLLYGFDTAYRGAITAPKATVSMNNAIWHLNSQSSINRLETKDSMVRFTGDNGKFTTLTVNNLTIDDSAFVLRANLAQADQLVVNKSLSGKNNLLLVDFIEKNGNSNGLNIDLVSAPKGTAVDVFKATTRSIGFSDVTPVIEQKNDTDKATWTLIGYKSVANADAAKKATLLMSGGYKAFLAEVNNLNKRMGDLRDINGESGAWARIISGTGSAGGGFSDNYTHVQVGADNKHELDGLDLFTGVTMTYTDSHAGSDAFSGETKSVGAGLYASAMFESGAYIDLIGKYVHHDNEYTATFAGLGTRDYSSHSWYAGAEVGYRYHVTDSAWIEPQAELVYGAVSGKQFSWKDQGMNLTMKDKDFNPLIGRTGVDVGKSFSGKDWKVTARAGLGYQFDLFANGETVLRDASGEKRIKGEKDGRMLMNVGLNAEIRDNLRFGLEFEKSAFGKYNVDNAINANFRYSF

SEQ ID NO: 6 - protein, 142 aa, Escherichia coli
MINIPSPTAVVMALVAISTLPSPSRVKLMPYPPRAHNTTGLLPVREICFPHHGDDGRNSIEPSISRAAHTDRLRFVCMTRTGSTTSRPFCPIPRSPALNASGQQDSGFWGVSSIPGDILMFQLHVLIVFICKINLSDNNISY

SEQ ID NO: 7 - protein, 318 aa, Escherichia coli
MYAREYRSTRPHKAIFFHLSCLTLICSAQVYAKPDMRPLGPNIADKGSVFYHFSATSFDSVDGTRHYRVWTAVPNTTAPASGYPILYMLDGNAVMDRLDDELLKQLSEKTPPVIVAVGYQTNLPFDLNSRAYDYTPAAESRKTDLHSGRFSRKSGGSNNFRQLLETRIAPKVEQGLNIDRQRRGLWGHSYGGLFVLDSWLSSSYFRSYYSASPSLGRGYDALLSRVTAVEPLQFCTKHLAIMEGSATQGDNRETHAVGVLSKIHTTLTILKDKGVNAVFWDFPNLGHGPMFNASFRQALLDISGENANYTAGCHELSH

SEQ ID NO: 8 - protein, 725 aa, Escherichia coli
MRINKILWSLTVLLVGLNSQVSVAKYSDDDNDETLVVEATAEQVLKQQPGVSVITSEDIKKTPPVNDLSDIIRKMPGVNLTGNSASGTRGNNRQIDIRGMGPENTLILIDGVPVTSRNSVRYSWRGERDTRGDTNWVPPEQVERIEVIRGPAAARYGSGAAGGVVNIITKRPTNDWHGSLSLYTNQPESSEEGATRRANFSLSGPLAGDALTTRLYGNLNKTDADSWDINSPVGTKNAAGHEGVRNKDINGVVSWKLNPQQILDFEVGYSRQGNIYAGDTQNSSSSAVTESLAKSGKETNRLYRQNYGITHNGIWDWGQSRFGVYYEKTNNTRMNEGLSGGGEGRILAGEKFTTNRLSSWRTSGELNIPLNVMVDQTLTVGAEWNRDKLDDPSSTSLTVNDRDISGISGSAADRSSKNHSQISALYIEDNIEPVPGTNIIPGLRFDYLSDSGGNFSPSLNLSQELGDYFKVKAGVARTFKAPNLYQSSEGYLLYSKGNGCPKDITSGGCYLIGNKDLDPEISVNKEIGLEFTWEDYHASVTYFRNDYQNKIVAGDNVIGQTASGAYILKWQNGGKALVDGIEASMSFPLVKERLNWNTNATWMITSEQKDTGNPLSVIPKYTINNSLNWTITQAFSASFNWTLYGRQKPRTHAETRSEDTGGLSGKELGAYSLVGTNFNYDINKNLRLNVGVSNILNKQIFRSSEGANTYNEPGRAYYAGVTASF

SEQ ID NO: 9 - protein, 1014 aa, Escherichia coli
MGNQWQQKYLLEYNELVSNFPSPERVVSDYIKNCFKTDLPWFSRIDPDNAYFICFSQNRSNSRSYTGWDHLGKYKTEVLTLTQAALINIGYRFDVFDDANSSTGIYKTKSADVFNEENEEKMLPSEYLHFLQKCDFAGVYGKTLSDYWSKYYDKFKLLLKNYYISSALYLYKNGELDEREYNFSMNALNRSDNISLLFFDIYGYYASDIFVAKNNDKVMLFIPGAKKPFLFKKNIADLRLTLKELIKDSDNKQLLSQHFSLYSRQDGVSYAGVNSVLHAIENDGNFNESYFLYSNKTLSNKDVFDAIAISVKKRSFSDGDIVIKSNSEAQRDYALTILQTILSMTPIFDIVVPEVSVPLGLGIITSSMGISFDQLINGDTYEERRSAIPGLATNAVLLGLSFAIPLLISKAGINQEVLSSVINNEGRTLNETNIDIFLKEYGIAEDSISSTNLLDVKLKSSGQHVNIVKLSDEDNQIVAVKGSSLSGIYYEVDIETGYEILSRRIYRTEYNNEILWTRGGGLKGGQPFDFESLNIPVFFKDEPYSAVTGSPLSFINDDSSLLYPDTNPKLPQPTSEMDIVNYVKGSGSFGDRFVTLMRGATEEEAWNIASYHTAGGSTEELHEILLGQGPQSSLGFTEYTSNVNSADAASRRHFLVVIKVHVKYITNNNVSYVNHWAIPDEAPVEVLAVVDRRFNFPEPSTPPDISTIRKLLSLRYFKESIESTSKSNFQKLSRGNIDVLKGRGSISSTRQRAIYPYFEAANADEQQPLFFYIKKDRFDNHGYDQYFYDNTVGLNGIPTLNTYTGEIPSDSSSLGSTYWKKYNLTNETSIIRVSNSARGANGIKIALEEVQEGKPVIITSGNLSGCTTIVARKEGYIYKVHTGTTKSLAGFTSTTGVKKAVEVLELLTKEPIPRVEGIMSNDFLVDYLSENFEDSLITYSSSEKKPDSQITIIRDNVSVFPYFLDNIPEHGFGTSATVLVRVDGNVVVRSLSESYSLNADASEISVLKVFSKKF

SEQ ID NO: 10 - protein, 454 aa, Escherichia coli
MVDMINESARQTPVIAQTDVLVIGGGPAGLSAAIAAGRLGARTMIVERYGSLGGVLTQVGVESFAWYRHPGTEDCEGICREYEGRARALGFTRPEPQSISEVIDTEGFKVVADQMITESGVEPLYHSWVVDVIKDGDTLCGVIVENKSGRGAILAKRIVDCTGDADIAARAGAPWTKRSKDQLMGVTVMFSCAGVDVARFNRFVAEELKPTYADWGKNWTIQTTGKEDPMFSPYMEDIFTRAQQDGVIPGDAQAIAGTWSTFSESGEAFQMNMVYAFGFDCTDVFDLTKAEIAGRQQALWAIDALRHYVPGFENVRLRNFGATLGTRESRLIEGEIRIADDYVLNQGRCSDSVGIFPEFIDGSGYLILPTTGRFFQIPYGCLVPQKVENLLVAGRCISAGVVAHTSMRNMMCCAVTGEAAGTAAVVSLQQNCTVRQVAIPDLQNTLQQQGVRLA

SEQ ID NO: 11 - protein, 253 aa, Escherichia coli
MSAKRRLLIACTLITAIYHFPAYSSLEYKGTFGSINAGYADWNSGFVNTHRGEVWKVTADFGVNFKEAEFYSFYESNVLNHAVAGRNHTVSAMTHVRLFDSDMTFFGKIYGQWDNSWGDDLDMFYGFGYLGWNGEWGFFKPYIGLHNQSGDYVSAKYGQTNGWNGYVVGWTAVLPFTLFDEKFVLSNWNEIELDRNDAYTEQQFGRNGLNGGLTIAWKFYPRWKASVTWRYFDNKLGYDGFGDQMIYMLGYDF

SEQ ID NO: 12 - protein, 492 aa, Escherichia coli
MASLIGLAVCTGNAFSPALAAEAKQPNLVIIMADDLGYGDLATYGHQIVKTPNIDRLAQEGVKFTDYYAPAPLSSPSRAGLLTGRMPFRTGIRSWIPSGKDVALGRNELTIANLLKAQGYDTAMMGKLHLNAGGDRTDQPQAQDMGFDYSLANTAGFVTDATLDNAKERPRYGMVYPTGWLRNGQPTPRADKMSGEYVSSEVVNWLDNKKDSKPFFLYVAFTEVHSPLASPKKYLDMYSQYMSAYQKQHPDLFYGDWADKPWRGVGEYYANISYLDAQVGKVLDKIKAMGEEDNTIVIFTSDNGPVTREARKVYELNLAGETDGLRGRKDNLWEGGIRVPAIIKYGKHLPQGMVSDTPVYGLDWMPTLAKMMNFKLPTDRTFDGESLVPVLEQKALKREKPLIFGIDMPFQDDPTDEWAIRDGDWKMIIDRNNKPKYLYNLKSDRYETLNLIGKKPDIEKQMYGKFLKYKTDIDNDSLMKARGDKPEAVTWG

SEQ ID NO: 13 - protein, 345 aa, Escherichia coli
LISLSFIPVMSALPGPIAKGFRNERGFVTTTICAMGELLAEFLSRNPHQKFTQPGEFIGPFPSGAPAIFAAQVAKLSHRAIFFGCVGNDDFARLIIERLRHEGVITDGIHVMNNAVTGTAFVSYQNPQQRDFVFNIPNSACGLFTAEHIDKDLLKQCNHLHIVGSSLFSFRMIDVMRKAITTIKSAGGTVSFDPNIRKEMLSIPEMAQALDYLIEYTDIFIPSESELPFFARHKNLSEEQIVSDLLHGGVKHVAIKRAQRGASYYKLKNGTLHAQHVAGHDIEIIDPTGAGDCFGATFITLFLSGFPAHKALQYANASGALAVMRQGPMEGISSLADIEDFLQQH

SEQ ID NO: 14 - protein, 192 aa, Escherichia coli
MYMPGKQMLCCILISIISEGDMKIFISLFLFIISTNSFADDITHAGVVRIEGLITEKTCIISDESKNFTVNMPDVPSSSVRSAGDVTEKVYFSITLTRCGSDVGNAYIKFTGNTVSEDASLYKLEDGSVEGLALTIFDKNKGSISNDVKSMVFSLTSSVDNILHFFAAYKALKNNVQPGDANASVSFIVTYD

SEQ ID NO: 15 - protein, 201 aa, Escherichia coli
MIKFRLYIPPVILGFVIVPLLVWPTVIALAVLIFTLTFLAEIIFSFPLLVVRISLQELQLELLVVYALFFSVMGGIGWQFSRRTPPELKNRLHCWLVFSPVYFWLILSNFILYISPEKSALLENIRNFFLTFVWLPLNFSPFWPQPWTDFVGPISAQLGFALGYYCQWRSKNRSHRKKWGDWVTCLSLAILALGPLFNYLQ

SEQ ID NO: 16 - protein, 234 aa, Escherichia coli
MKFNLSNLSAVLLASGMLMSTAVTAAPGDATQFGGADTDWSTVDYPRLTDMDDNVDSMGGKIRFTGRVVKATCKVATDSKQIEVVLPVVPSNLFTGIDVEAQGASNQTDFNINLTECSNTDDQKIEFRFTGTADSANKTLANEVEGSTDADNSGNAGATGVGIRIYSKGTTNNGLINLNTTAAEGSASTAAYTIPGNATTHDFSAAFTAGYAQNGSTVAPGVVKSTASFVVLYE

SEQ ID NO: 17 - protein, 336 aa, Escherichia coli
MRIHTYWYRRYFILLIIIFSNVLSSIANAEDMGRERAYCYPGSPSNNTTPASFSYNFGTIVVSDVNKNAPGTVLPSQIWKVGTYKAYCNSLDDYEIYFSAVSGIDPSGASGDHQGSDVFIPLTHEISVSTHIKLYNQNGTMTDKIVPFENYNTNYPGDRSKPSNWASGTEGYIKIRIDKKIISDVSLSNVLLVSLYVSQIPTEHGPIPVFNAYIGNLNIQVPQGCTINEGTSFTVNMPDVWASELSRAGAGAKPAGVTPVATTIPINCTNKDTDAVMTLVFDGNISATRDTNGKQSIIQAQDNPDVGIMIMDSQQNSVDLNALATSVGVPFRLVEN

SEQ ID NO: 18 - protein, 864 aa, Escherichia coli
MNLKLKRCEYWMAAQKQMKRVVPLLLVIMPACSIAGMRFNPAFLSGDTEAVADLSRFEKGMTYLPGSYEVEVWVNDSPLLSRTVTFKADDENQLIPCLSLADLLSLGINKNALPEQALASSENSCLDLRIWFPDVHYMPELDAQRLKLTFPQAIIKRDARGYIPPEQWDNGITAFLLNYDFSGNNDRGDYSSNNYYLNLRAGINIGAWRFRDYSTWSRGSNSAGKLEHISSTLQRVIIPFRSELTLGDTWSSSDVFDSVSIRGIKLESDENMLPDSQSGFAPTVRGIAKSRAQVTIKQNGYVIYQTYMPPGPFEISDLNPTSSAGDLEVTIKESDNSETVYTVPYAAVPILQREGHLKYSTTVGQYRSNSYNQKSPYVFQGELIWGLPWDITAYGGAQFSEDYRALALGLGLNLGVFGATSFDVTQANSSLVDGSKHQGQSYRFLYSKSLVQTGTAFHIIGYRYSTQGFYTLSDTTYQQMSGTVVDPKTLDDKDYVYNWNDFYNLRYSKRGKFQASVSQPFGNYGSMYLSASQQTYWNTDKKDSLYQVGYNTSIKGIYLNVAWNYSKSPGTNADKIVSLNVSLPISNWLSSTNDGRSSSNAMTATYGYSQDNHGQVNQYTGVSGSLLEQHNLSYNIQHGFANQDNSSSGSVGVNYRGAYGSLNSAYSYDNEGNQQINYGISGALVVHENGLTLSQPLGETNVLIKAPGANNVDVQRGTGISTDWRGYAVVPYATEYRRNNISLDPMSMNMHTELDITSTEVIPGKGALVRAEFAAHIGIRGLFTVRYRNKSVPFGATASAQIKNSSQITGIVGDNGQLYLSGLPLEGVINIQWGDGVQQKCQANYKLPETELDNPVSYATLECR

SEQ ID NO: 19 - protein, 169 aa, Escherichia coli
MGAIYVKRLILSVALIIPIASNASDALNQPSSSLNDGVETFFISCFDMPQETTTDMDACQRVQLAQVSWVKNKYSVAALNRLKQDNKDDPQRLQELTASFNAESEAWTELIEKASKSVQVDYVGGTIAGTAVASRQIGLLELQSHDIWEHWLRSRGLNSSSFARTKVQI

SEQ ID NO: 20 - protein, 713 aa, Escherichia coli
MAMFTPSFSGLKGRALFSLLFAAPMIHATDSVTTKDGETITVTADANTATEATDGYQPLSTSTATLTDMPMLDIPQVVNTVSDQVLENQNATTLDEALYNVSNVVQTNTLGGTQDAFVRRGFGANRDGSIMTNGLRTVLPRSFNAATERVEVLKGPASTLYGILDPGGLINVVTKRPEKTFHGSVSATSSSFGGGTGQLDITGPIEGTQLAYRLTGEVQDEDYWRNFGKERSTFIAPSLTWFGDNATVTMLYSHRDYKTPFDRGTIFDLTTKQPVNVDRKIRFDEPFNITDGQSDLAQLNAEYHLNSQWTARFDYSYSQDKYSDNQARVTAYDATTGTLTRRVDATQGSTQRMHATRADLQGNVDIAGFYNEILGGVSYEYYDLLRTDMIRCKKAKDFNIYNPVYGNTSKCTTVSASDSDQTIKQENYSAYAQDALYLTDNWIAVAGIRYQYYTQYAGKGRPFNVNTDSRDEQWTPKLGLVYKLTPSVSLFANYSQTFMPQSSIASYIGDLPPESSNAYEVGAKFELFDGITADIALFDIHKRNVLYTESIGDETIAKTAGRVRSRGVEVDLAGALTENINIIASYGYTDAKVLEDPDYAGKPLPNVPRHTGSLFLTYDIHNMPGNNTLTFGGGGHGVSRRSATNGADYYLPGYFVADAFAAYKMKLQYPVTLQLNVKNLFDKTYYTSSIATNNLGNQIGDPREVQFTVKMEF

SEQ ID NO: 21 - protein, 606 aa, Escherichia coli
MKISWNYIFKNKWRFHITSISLFLIMLAVSIAFLHLRFNTLSSTDKMRLEMYKSTLYSTIEQFYVLPYMLSTDHIIRQAVITPDDMTSSELNQRIAHFNTQLKTAAIFILDTQGKAIASSNWQDPGSYVGQNYSYRPYYKHAMSGLNGRFYGIGSTTNTPGFFLSTSIKDKGKIVGVVVVKISLNEIEKAWAEGPENIIVNDEHGIIFLSSKSPWRMRTLQPLPVQAKQKLQSTRQYSLDNLLPADYYPCYTVSNFTFLKDKKEQLCLFPQYYTQQIAIPEFNWKMTIMVPLDNLYWSWAISLVITLIIYLLFLLFIKYWRMRSHAQQLLTLANETLEKQVKERTSALELINQKLIQEIKERSQAEQVLQITRSELAESSKLAALGQMATEIAHEQNQPLAAIHALTDNARTMLKKEMYPQVEQNLKHIISVIERMTQLISELKAFASRHRVPKGSADVIKVMYSAVALLNHSMEKNNIERRIKAPSMPLFVNCDELGLEQIFSNLISNALDSMEGSSYKRLDIAIRQANNKVIITIKDSGGGFAPEVVDRIFEPFFTTKRRGMGLGLAIVSEIVRNSNGALHASNHPEGGAVMTLTWPEWGEEHE

SEQ ID NO: 22 - protein, 101 aa, Escherichia coli
VLTPQHLRCVLTCSDLLTLLSGTVMSQMPLYFLNTQKKLTAHYEWLQINLTDTYELVKRLMPIPSLDVVVKVGKLVLPEKGHHGFYPEAGVVYRTVAPENP

SEQ ID NO: 23 - protein, 263 aa, Escherichia coli
MMKNTGYILALCLTASGHVLAHDVWITGKQAENNVTAEIGYGHNFPSKGTIPDRRDFFENPRLYNGKETITLKPASTDYVYKTESASKDNGYVLSTYMKPGYWSRTSSGWKPVSREGRNDVAYCEFVTKYAKSFIPGEQQMPAQLYQSPTGHELEIIPLSDISRFSENVKLKVLYKTSPLAGAIMELDSVSYLTSSRHTHAVEHKHPVHKAELTFVTNEDGIVTVPSLHIGQWLAKVQNKKSFQDKSLCDETVDVATLSFSRN

SEQ ID NO: 24 - protein, 378 aa, Escherichia coli
MGKIKYWLIVGFIILFAIFYIAISDRDSTLSRLKSAGENGDVEAQYALGLMYLYGEILDVDYQQAKIWYEKAADQNDPRAQAKLGVMYANGLGVNQDYQQSKLWYEKAAAQNDVDAQFLLGEMYDDGLGVSQDYQHAKMWYEKAAAQNDERAQVNLAVLYAKGNGVEQDYRQAKSWYEKAAAQNSPDAQFALGILYANANGVEQDYQQAKDWYEKAAEQNFANAQFNLGMLYYKGEGVKQNFRQAREWFEKAASQNQPNAQYNLGQIYYYGQGVTQSYRQAKDWFEKAAEKGHVDAQYNLGVIYENGEGVSQNYQQAKAWYEKAASQNDAQAQFELGVMNELGQGESIDLKQARHYYERSCNNGLKKGCERLKELLYK

SEQ ID NO: 25 - protein, 654 aa, Escherichia coli
MNVIRTVICTLIILPVGLQAATSHSSMVKDTITIVATGNQNTVFETPSMVSVVTNDTPWSQNAVTSAGMLKGVAGLSQTGAGRTNGQTFNLRGYDKSGVLVLVDGVRQLSDMAKSSGTYLDPALVKRIEVVRGPNSSLYGSGGLGGVVDFRTADAADFLPPGETNGLSLWGNIASGDHSTGSGLTWFGKTGKTDALLSVIMRKRGNIYQSDGEHAPNKEKPAALFAKGSVGITDSNKAGASLRLYRNNTTEPGNSTQTHGDSGLRDRKTVQNDVQFWYQYAPVDNSLINVKSTLYLSDITIKTNGHNKTAEWRNNRTSGVNVVNRSHTLIFPGAHQLSYGAEYYRQQQKPEGSATLYPEGNIDFTSLYFQDEMTMKSYPVNIIVGSRYDRYKSFNPRAGELKAERLSPRAAISVSPTDWLMMYGSISSAFRAPTMAEMYRDDVHFYRKGKPNYWVPNLNLKPENNITREIGAGIQLDGLLTDNDRLQLKGGYFGTDARNYIATRVDMKRMRSYSYNVSRARIWGWDMQGNYQSDYVDWMLSYNRTESMDASSREWLGSGNPDTLISDISIPVGHRGVYAGWRAELSASATHVKKGDPHQAGYTIHSFSLSYKPVSVKGFEASVTLDNAFNKLAMNGKGVPLSGRTVSLYTRYQW

SEQ ID NO: 26 - protein, 1376 aa, Escherichia coli
MNKIYALKYCYITNTVKVVSELARRVCKGSTRRGKRLSVLTSLALSALLPTVAGASTVGGNNPYQTYRDFAENKGQFQAGATNIPIFNNKGELVGHLDKAPMVDFSSVNVSSNPGVATLINPQYIASVKHNKGYQSVSFGDGQNSYHIVDRNEHSSSDLHTPRLDKLVTEVAPATVTSSSTADILNPSKYSAFYRAGSGSQYIQDSQGKRHWVTGGYGYLTGGILPTSFFYHGSDGIQLYMGGNIHDHSILPSFGEAGDSGSPLFGWNTAKGQWELVGVYSGVGGGTNLIYSLIPQSFLSQIYSEDNDAPVFFNASSGAPLQWKFDSSTGTGSLKQGSDEYAMHGQKGSDLNAGKNLTFLGHNGQIDLENSVTQGAGSLTFTDDYTVTTSNGSTWTGAGIIVDKDASVNWQVNGVKGDNLHKIGEGTLVVQGTGVNEGGLKVGDGTVVLNQQADSSGHVQAFSSVNIASGRPTVVLADNQQVNPDNISWGYRGGVLDVNGNDLTFHKLNAADYGATLGNSSDKTANITLDYQTRPADVKVNEWSSSNRGTVGSLYIYNNPYTHTVDYFILKTSSYGWFPTGQVSNEHWEYVGHDQNSAQALLANRINNKGYLYHGKLLGNINFSNKATPGTTGALVMDGSANMSGTFTQENGRLTIQGHPVIHASTSQSIANTVSSLGDNSVLTQPTSFTQDDWENRTFSFGSLVLKDTDFGLGRNATLNTTIQADNSSVTLGDSRVFIDKKDGQGTAFTLEEGTSVATKDADKSVFNGTVNLDNQSVLNINEIFNGGIQANNSTVNISSDSAVLENSTLTSTALNLNKGANVLASQSFVSDGPVNISDATLSLNSRPDEVSHTLLPVYDYAGSWNLKGDDARLNVGPYSMLSGNINVQDKGTVTLGGEGELSPDLTLQNQMLYSLFNGYRNTWSGSLNAPDATVSMTDTQWSMNGNSTAGNMKLNRTIVGFNGGTSSFTTLTTDNLDAVQSAFVMRTDLNKADKLVINKSATGHDNSIWVNFLKKPSDKDTLDIPLVSAPEATADNLFRASTRVVGFSDVTPTLSVRKEDGKKEWVLDGYQVARNDGQGKAAATFMHISYNNFITEVNNLNKRMGDLRDINGEAGTWVRLLNGSGSADGGFTDHYTLLQMGADRKHELGSMDLFTGVMATYTDTDASAGLYSGKTKSWGGGFYASGLFRSGAYFDLIAKYIHNENKYDLNFAGAGKQNFRSHSLYAGAEVGYRYHLTDTTFVEPQAELVWGRLQGQTFNWNDSGMDVSMRRNSVNPLVGRTGVVSGKTFSGKDWSLTARAGLHYEFDLTDSADVHLKDAAGEHQINGRKDGRMLYGVGLNARFGDNTRLGLEVERSAFGKYNTDDAINANIRYSF

SEQ ID NO: 27 - protein, 349 aa, Escherichia coli
MITLFRLLAILCLFFNVSAFAVDCYQDGYRGTTLINGDLPTFKIPENAQPGQKIWESGDINITVYCDNAPGWSSNNPSENVYAWIKLPQINSADMLNNPYLTFGVTYNGVDYEGTNEKIDTHACLDKYEQYYNGYYHDPVCNGSTLQKNVTFNAHFRVYVKFKSRPAGDQTVNFGTVNVLQFDGEGGANMAPNAKNLRYAITGLDNISFLDCSVDVRISPESQIVNFGQIAANSIATFPPKAAFSVSTIKDIASDCTEQFDVATSFFTSDTLYDNTHLEIGNGLLMRITDQKTQEDIKFNQFKLFSTYIPGQSAAMATRDYQAELTQKPGEPLVYGPFQKDLIVKINYH

SEQ ID NO: 28 - protein, 840 aa, Escherichia coli
MNNKNTFSRDKLSHAIKNALSGVVCSLLFVLPVHAVEFNVDMIDAEDRENIDISRFEKKGYIPPGRYLVRVQINKNMLPQTLILEWVKADNESGSLLCLTKENLTNFGLNTEFIESLQNIAGSECLDLSQRQELTTRLDKATMILSLSVPQAWLKYQATNWTPPEFWDTGITGFILDYNVYASQYAPHHGDSTQNVSSYGTLGFNLGAWRLRSDYQYNQNFADGRSVNRDSEFARTYLFRPIPSWSSKFTMGQYDLSSNLYDTFHFTGASLESDESMLPPDLQGYAPQITGIAQTNAKVTVAQNGRVLYQTTVAPGPFTISDLGQSFQGQLDVTVEEEDGRTSTFQVGSASIPYLTRKGQVRYKTSLGKPTSVGHNDINNPFFWTAEASWGWLNNVSLYGGGMFTADDYQAITTGIGFNLNQFGSLSFDVTGADASLQQQNSGNLRGYSYRFNYAKHFESTGSQITFAGYRFSDKDYVSMSEYLSSRNGDESIDNEKESYVISLNQYFETLELNSYLNVTRNTYWDSASNTNYSVSVSKNFDIGDFKGISASLAVSRIRWDDDEENQYYFSFSLPLQQNRNISYSMQRTGSSNTSQMISWYDSSDRNNIWNISASATDDNIRDGEPTLRGSYQHYSPWGRLNINGSVQPNQYNSVTAGWYGSLTATRHGVALHDYSYGDNARMMVDTDGISGIEINSNRTVTNGLGIAVIPSLSNYTTSMLRVNNNDLPEGVDVENSVIRTTLTQGAIGYAKLNATTGYQIVGVIRQENGRFPPLGVNVTDKATGKDVGLVAEDGFVYLSGIQENSILHLTWGDNTCEVTPPNQSNISESAIILPCKTVK

SEQ ID NO: 29 - protein, 169 aa, Escherichia coli
LMNTKQSVAQLAVPHRKRLSSTMVVALLLCVVAGAVMINAADFPATAIETDPGASAFPTFYACALIVLAVLLVIRDLLQAKPASCANAQEKPAFRKTATGIAATAFYIVAMSYCGYLITTPVFLIVIMTLMGYRRWVLTPGIALLLTAILWLLFVEALQVPLPVGTFFE

SEQ ID NO: 30 - protein, 311 aa, Escherichia coli
MVLLAGAALSIAPVQAASYPTKQIELVVPYAAGGGTDLVARAFADAAKNHLPVSIGVINKPGGGGAIGLSEIAAARPNGYKIGLGTVELTTLPSLGMVRFKTSDFKPIARLNADPAAITVRADAPWNSYEEFMAYSKANPGKVRIGNSGTGAIWHLAAAALEDKTGTKFSHVPYDGAAPAITGLLGGHIEAVSVSPGEVINHVNGGKLKTLVVMADERMKTMPDVPTLKEKGVDLSIGTWRGLIVSQKTPQDVVDVLAKAAKETAEEPAFQDALQKLNLNYAWLDAASFQTQISEQEKYFDELLTRLGLKK

SEQ ID NO: 31 - protein, 722 aa, Escherichia coli
MLRWKRCIILTFISGAAFAAPEINVKQNESLPDLGSQAAQQDEQTNKGKSLKERGADYVINSATQGFENLTPEALESQARSYLQSQITSTAQSYIEDTLSPYGKVRLNLSIGQGGDLDGSSIDYFVPWYDNQTTVYFSQFSAQRKEDRTIGNIGLGVRYNFDKYLLGGNIFYDYDFTRGHRRLGLGAEAWTDYLKFSGNYYHPLSDWKDSEDFDFYEERPARGWDIRAEVWLPSYPQLGGKIVFEQYYGDEVALFGTDNLEKDPYAVTLGLNYQPVPLLTVGTDYKAGTGDNSDVSINATLNYQFGVPLKDQLDSDKVKAAHSLMGSRLDFVERNNFIVLEYKEKDPLDVTLWLKADATNEHPECVIKDTPEAAVGLEKCKWTINALINHHYKIVAASWQAKNNAARTLVMPVIKENTLTEGNNNHWNLVLPAWQYSSDQAEQEKLNTWRVRLALEDEKGNRQNSGVVEITVQQDRKIELIVNNIANPEENNHSHEASAQADGVDGVVMDLDVTDSFGDNTDRNGDALPEDNLTPQLYDAQDKRVTLTNKPCSTDNPCVFIAKQDKEKGTVTLSSTLPGTYRWKAKAAPYDDSNYVDVTFLGAEIGGLNAFIYRVGAAKPSNLIGKDKEPLPSTTFIDLFYGATTIKTVSSSRSKNLTKRWCSTTTSGNLPARASMVSGCTGEHSNEDIVIPATNREAAQTYGAQAGDGLQGYGLRVLYTKK

SEQ ID NO: 32 - protein, 319 aa, Escherichia coli
MKQDKRRGLTRIALALALAGYCVAPVALAEDSAWVDSGETNIFQGTIPWLYSEGGSATTDADRVTLTSDLKGARPQGMKRTSVFTRVINIGDTEGDVDLGGLGDNAKTIDTIRWMSYKDAQGGDPKELATKVTSYTLTDADRGRYIGIEITPTTQTGTPNVGTALHLYDVSTASGGGSDSDNVAPGPVVNQNLKVAIFVDGTSINLINGSTPIELGKTYVAKLYSDENKNGKFDAGTDADVTANYDFRWVLSGSSQQLGTSGGIVNSSFDNNNLVIPATNDEARTNLNGPARDGKEALSIPTNGDGVQGYKLHIIYKHK

SEQ ID NO: 33 - protein, 629 aa, Escherichia coli
MKKVLTLSLLALCVSHSAVAANYTFNNDNIALSFDDTNSTIVLKDRRTNHPITPQELFFLTLPDETKIHTADFKIKHIKKQDNAIVIDFTRPDFNVTVQLNLVKGKYASIDYTIAAVGQPRDVAKITFFPTKKQFQAPYVDGAITSSPIIADSFFILPNKPIVNTYAYEATTNLNVELKTPIQPETPVSFTTWFGTFPETSQLRRSVNQFINAVRPRPYKPYLHYNSWMDIGFFTPYTEQDVLGRMDEWNKEFISGRGVALDAFLLDDGWDDLTGRWLFGPAFSNGFSKVREKADSLHSSVGLWLSPWGGYNKPQRRSRFACKRVWVRNRGRQAGAFGSELLKNFNEQIINLIKNEHITSFKLDGMGNASSHIKGSPFASDFDASIALLHNMRRANPNLFINLTTGTNASPSWLFYADSIWRQGDDINLYGPGTPVQQWITYRDAETYRSIVRKGPLFPLNSLMYHGIVSAENAYYGLEKVQTDSDFADQVWSYFATGTQLQELYITPSMLNKVKWDTLAKAAKWSKENASVLVDTHWIGGDPTALAVYGWASWSKDKAILGLRNPSDKPQTYYLDLAKDFEIPAGNAAQFSLKAVYGSNKTVPVEYKNATVITLQPLETLVFEAVTIN

SEQ ID NO: 34 - protein, 1778 aa, Escherichia coli
MNKIFKVIWNPATGSYTVASETAKSRGKKSGRSKLLISALVAGGLLSSFGASADNYTGQPTDYGDGSAGDGWVAIGKGAKANTFMNTSGASTALGYDAIAEGEYSSAIGSKTLATGGASMAFGVSAKAMGDRSVALGASSVANGDRSMAFGRYAKTNGFTSLAIGDSSLADGEKTIALGNTAKAYEIMSIALGDNANASKEYAMALGASSKAGGADSLAFGRKSTANSTGSLAIGADSSSSNDNAIAIGNKTQALGVNSMALGNASQASGESSIALGNTSEASEQNAIALGQGSIASKVNSIALGSNSLSSGENAIALGEGSAAGGSNSLAFGSQSRANGNDSVAIGVGAAAATDNSVAIGAGSTTDASNTVSVGNSATKRKIVNMAAGAISNTSTDAINGSQLYTISDSVAKRLGGGATVGSDGTVTAVSYALRSGTYNNVGDALSGIDNNTLQWNKTAGAFSANHGANATNKITNVAKGTVSATSTDVVNGSQLYDLQQDALLWNGTAFSAAHGTEATSKITNVTAGNLTAGSTDAVNGSQLKTTNDNVTTNTTNIATNTTNITNLTDAVNGLGDDSLLWNKAAGAFSAAHGTEATSKITNVTAGNLTAGSTDAVNGSQLKTTNDNVTTNTTNIATNTTNITNLTDAVNGLGDDSLLWNKTAGAFSAAHGTDATSKITNVTAGNLTAGSTDAVNGSQLKTTNDNVTTNTTNIATNTTNITNLTDAVNGLGDDSLLWNKTAGAFSAAHGTDATSKITNVKAGDLTAGSTDAVNGSQLKTTNDNVSTNTTNITNLTDAVNGLGDDSLLWNKTAGAFSAAHGTDATSKITNVKAGDLTAGSTDAVNGSQLKTTNDNVSTNTTNITNLTDSVGDLKDDSLLWNKAAGAFSAAHGTEATSKITNLLAGKISSNSTDAINGSQLYGVADSFTSYLGGGADISDTGVLSGPTYTIGGTDYTNVGDALAAINTSFSTSLGDALLWDATAGKFSAKHGINNAPSVITDVANGAVSSTSSDAINGSQLYGVSDYIADALGGNAVVNTDGSITTPTYAIAGGSYNNVGDALEAIDTTLDDALLWDTTANGGNGAFSAAHGKDKTASVITNVANGAVSATSNDAINGSQLYSTNKYIADALGGDAEVNADGTITAPTYTIANTDYNNVGEALDALDNNALLWDEDAGAYNASHDGNASKITNVAAGDLSTTSTDAVNGSQLNATNILVTQNSQMINQLAGNTSETYIEENGAGINYVRTNDSGLAFNDASASGIGATAVGYNAVASHASSVAIGQDSISEVDTGIALGSSSVSSRVIVKGTRNTSVSEEGVVIGYDTTDGELLGALSIGDDGKYRQIINVADGSEAHDAVTVRQLQNAIGAVATTPTKYYHANSTAEDSLAVGEDSLAMGAKTIVNGNAGIGIGLNTLVLADAINGIAIGSNARANHADSIAMGNGSQTTRGAQTNYTAYNMDAPQNSVGEFSVGSEDGQRQITNVAAGSADTDAVNVGQLKVTDAQVSQNTQSITNLNTQVTNLDTRVTNIENGIGDIVTTGSTKYFKTNTDGADANAQGKDSVAIGSGSIAAADNSVALGTGSVADEENTISVGSSTNQRRITNVAAGVNATDAVNVSQLKSSEAGGVRYDTKADGSIDYSNITLGGGNSGTTRISNVSAGVNNNDAVNYAQLKQSVQETKQYTDQRMVEMDNKLSKTESKLSGGIASAMAMTGLPQAYTPGASMASIGGGTYNGESAVALGVSMVSANGRWVYKLQGSTNSQGEYSAALGAGIQW

SEQ ID NO: 35 - protein, 227 aa, Escherichia coli
MNLKKTLLSVLMILQLCLLVGCDYIEKASKVDDLVTQQELQKSKIEALEKQQELDKRKIEHFEKQQTTIINSTKTLAGVVKAVKNKQDEFVFTEFNPAQTQYFILNNGSVGLAGKILSIDAVENGSVIRISLVNLLSVPVSNMGFYATWGGEKPTDINALAKWQQLLFSTAMNSSLKLLPGQWQDINLTLKGVSPNNLKYLKLAINMANIQFDRLQPAESPQRKNKK

SEQ ID NO: 36 - protein, 1109 aa, Escherichia coli
MKRVVRLLGVGLLLLVVLLLILFVLAQTTPLISAQDEHAVWLRLLITAIVICLLSMCIFFLFSFRQNEASTISLYAQPTDIKEINTEQPNYASLLTIYLRDRYGPFWRRKVRLLLVTGEPEQAEAIAPGLTGQHWLEGDHTVLIYGGRPTAEPDVTLLTALKKLRRSRPLDGIIWALTEEQSRQTAQLDKGWRGLINGGKRLGFQAPLYLWQVCDDGDYQTGRPLQSVGCLLPERCTPEQLAVMLEAAADGTGHVAATDRYRMFSAASGSYPCRAGYCSLADRPETAAGRRRIFFPAPARPDVQPAACRRAGGQHLMQWLPSPVWAGVTVITRAGARWVFLWLRTALMSAVCVLVIWGAGMTTSFFANRALVQETGIQTARALDTRLPLAEQLVALHTLQGELERLQYRIREGAPWYQRFGLERNQQLLAAAFPGYAQAANRLVRDVAVDHLQQQLNAFVALPPNSPQRTATGEQRYKQLKALLMTSRPEKADAAFFSTTLMADGLRYENIPEGVRQSVLPSLLTFWTANLPEHPQWKTSPPPELTGAVRKILLRQIGVRNAENTLYQNVLQQVSRNYADMTLADMTGDTLTESLFSTEQTVPGMFTRQAWEGQVREAIEQVVTARREEIDWVLSDRQQDTSADISPDTLRNRLTSRYFTDFAGSWLAFLNSIHWKKEDSLSGILDQLTLMADARQSPLIALTDTLAWQAATGRENRGLSDSLAKSAQELFNGKEKTPQQSREGDDVPVGPLDKTFTPLLRLLGDKAGGGDSQLSLQTYLTRVTRVRLKLQQVTNAPDPQEMTQQLAQTVLQGKTVDLTDTRDYGRLIAASLGEEWSGFGQALFVRPVEQSWRQVLTPAADSLNRQWQRAIVSHWNQDFAGRYPFKASQNDASLPLLAQYLRDDGRINLFIAANLSGVLKREGRYWVADAMNTQGLTVNPDFIRALNRLRDVADTAFASGDAGIHFELRAKPARDVMKTHLVIDGQELEYFNQKERWQRFNWPDEQWQPGASLSWTSTQAMERILADYRGSWSLIRLLEQAQVTPVDSSTFKVVWKAQDGLPLNYLLRVEQGKGPLALLELKNFRLPGQVFLTGKSMKDVEEYGEDADE

SEQ ID NO: 37 - protein, 178 aa, Escherichia coli
MFPIRFKRPALLCMAMLTVVLSGCGLIQKVVDESKSVASAVFYKQIKILHLDFFSRSALNTDAEDTPLSTMVHVWQLKTREDFDKADYDTLFMQEEKTLEKDVLAKHTVWVKPEGTASLNVPLDKETQFVAIIGQFYHPDEKSDSWRLVIKRDELEADKPRSIELMRSDLRLLPLKDK

SEQ ID NO: 38 - protein, 280 aa, Escherichia coli
MISGGNMLKEWMIFTCSLLTLAGASLPLSGCISRGQESISEGAAFGAGILREPGATKKADTKDLNVPPPVYGPPQVIFRIDDNRYFTLENYTHCENGQTFYNNKAKNIHVKILDASGYLFKGRLFWLSTRDDFLAFPATLNTRHASCMGSNKGCMNAVIVTTDGGKRRSGVPYGSYTQNPTGATRDYDMLVMNDGFYLLRYRGGQGRFSPVILRWILSTEDSSGVVRSEDAYELFRPGEEVPSTGFYKIDLSRFYPKNNVMEMQCDRTLEPVQPSESKIQ

SEQ ID NO: 39 - protein, 501 aa, Escherichia coli
MEHVSIKTLYHLLCCMLLFISAMCALAQEHEPIGAQDERLSTLIHQRMQEAKVPALSVSVTIKGVRQRFVYGVADVASQKANTLDTVYELGSMSKAFTGLVVQILIQEGRLRQGDDIITYLPEMRLNYQGKPASLTVADFLYHTSGLPFSTLARLENPMPGSAVAQQLRNENLLFAPGAKFSYASANYDVLGAVIENVTGKTFTEVIAERLTQPLGMSATVAVKGDEIIVNKASGYKLGFGKPVLFHAPLARNHVPAAYIHSTLPDMEIWIDAWLHRKALPATLREAMSNSWRGNSDVPLAADNRILYASGWFIDQNQGPYISHGGQNPNFSSCIALRPDQQIGIVALANMNSNLILQLCADIDNYLRIGKYADGAGDAITATDTLFVYLTLLLCFWGAVVVVRGAFRVYRATAHGPGKQQRLRLRVRDYIIALAVPGLVAAMLYVAPGILSPGLDWRFILVWGPSSVLAIPFGIILLAFVLTLNHQIKRILLHNKEWDDE

SEQ ID NO: 40 - protein, 682 aa, Escherichia coli
MKNKYIIAPGIAVMCSAVISSGYASSDKKEDTLVVTASGFTQQLRNAPASVSVITSEQLQKKPVSDLVDAVKDVEGISITGGNEKPDISIRGLSGDYTLILVDGRRQSGRESRPNGSGGFEAGFIPPVEAIERIEVIRGPMSSLYGSDAIGGVINIITKPVNNQTWDGVLGLGGIIQEHGKFGNSTTNDFYLSGPLIKDKLGLQLYGGMNYRKEDSISQGTPAKDNKNITATLQFTPTESQKFVFEYGKNNQVHTLTPGESLDAWTMRGNLKQPNSKRETHNSRSHWVAAWNAQGEILHPEIAVYQEKVIREVKSGKKDKYNHWDLNYESRKPEITNTIIDAKVTAFLPENVLTIGGQFQHAELRDDSATGKKTTETQSVSIKQKAVFIENEYAATDSLALTGGLRLDNHEIYGSYWNPRLYAVYNLTDNLTLKGGIAKAFRAPSIREVSPGFGTLTQGGASIMYGNRDLKPETSVTEEIGIIYSNDSGFSASATLFNTDFKNKLTSYDIGTKDPVTGLNTFIYDNVGEANIRGVELATQIPVYDKWHVSANYTFTDSRRKSDDESLNGKSLKGEPLERTPRHAANAKLEWDYTQDITFYSSLNYTGKQIWAAQRNGAKVPRVRNGFTSMDIGLNYQILPDTLINFAVLNVTDRKSEDIDTIDGNWQVDEGRRYWANVRVSF

SEQ ID NO: 41 - protein, 164 aa, Escherichia coli
MGFRKTIITSVGLIFISFSFVAKCSQLKNLNNYSVMLCGKVSNNILDDIGGYKERNILMLRAIKKIIIMTIVNIIFFYSFQSTADEMVLIKKYGFGLERDIKGRPLIYPIENYDECKKKCNHMNYIADVNAQLAMSKKNNRIFANITFTNNSSTTYFFLNIIYL

SEQ ID NO: 42 - protein, 218 aa, Escherichia coli
MNQIKDNKVIMKIKNLISVILLSGGIMGTGLYSSDNHQKIRSRFNIQESYCAIKTNGVLGFSNRKDVLRENGDSTGTTSSSTNAMMLMENGENEISLEIGALRWFSDKPASTEERGHFSQKAGCSLDLVRFVKQEETILSSIKVTINQQGIPEAQPDSMHPVIRKEILAEQAEPGFIDPDYFNETYFPKGMKVYQFTQKVSVAGLPDGPGRSTPFTGA

SEQ ID NO: 43 - protein, 2732 aa, Escherichia coli
MHQPPVRFTYRLLSYLVSAIIAGQPLLPAVGAVITPQNGAGMDKAANGVPVVNIATPNGAGISHNRFTDYNVGKEGLILNNATGKLNPTQLGGLIQNNPNLKAGGEAKGIINEVTGGKRSLLQGYTEVAGKAANVMVANPYGITCDGCGFINTPHATLTTGKPVMNADGSLQALEVTEGSITINGAGLDGTRSDAVSIIARATEVNAALHAKDLTVTAGANRVTADGRVRALKGEGDVPKVAVDTGALGGMYARRIHLTSTESGVGVNLGNLYARDGDITLDASGRLTVNNSLATGAVTAKGQGVTLTGDHKAGGNLSVSSRRDIVLSNGTLNSDKDLSLTAGGRITQQNEKLTAGRDVTLAAKNITQDTASQINAARDIVTVASDTLTTQGQITAGQNLTASATTLTQDGILLAKSHAGLNAGTLNNSGAVQGATLTLGSTTLSNSGSLLSGGPLTMNTRDFTQSGRTGAKGKVDIMASGKLTSTGLLVTMHLVLKAQDVTQNGVLSGGKGLTVSATSSGKKSVTHSDAAMTLNVTTVALDGETSAGDTLRVQADKLSTAAGAQLQSGKNLSINARDARLAGTQAAQQTMVVNASEKLTHSGKSSAPSLSLSAPELTSSGVLVGSALNTQSQTLTNSGLLQGEASLTVNTQRLDNQQNGTLYSAADLTLDIPDIRNSGLITGDNGLMLNAVSLSNPGKIIADTLSVRATTLDGDGLLQGAGALALAGDTLSQGSHGRWLTADDLSLRGKTLNTAGTTQGQNITVQADRWANSGSVLATGNLTASATGQLTSTGDIMSQGDTTLKAATTDNRGSLLSAGTLSLDGNSLDNRGTVQGNHVTIRQNSVTNSGTLTGIAALTLAARMASPQPALMNNGGSLLTSGDLTITAGSITSSGHWQGKRVLITADSLANSGAIQAADSLTARLTGELVSTAGSKVTSNGEMALSALNLSNSGQWIAKNLTLKANSLTSAGDITGVDTLTLTVNQTLNNQANGKLLSAGVLTLKADSVTNDGQLQGNVTTITAGQLTNGGHLQGETLTLTASGGVNNRSGGVLMSRNALNVSTATLSNQSTIQGGGGVSLNATDRLQNDGKILSGSNLTLTAQVLANTGSGLVQAATLLLDVVNTVNGGRVLATGSDVKGTTLNNTGTLQGATLVNYHTFSSGTLLGTSGLGVKGSSLLQNGTGRLYSAGNLLLDAQDFSGQGQVVATGDVTLKLIAALTNHGTLAAGKTLSVTSQNAITNGGVMQGDAMVLGAGEAFTNNGLTAGKGNSVFSAQRLFLNAPGSLQGGGDVSLNSRSDITISGFTGTAGSLTMNVAGTLLNSALIYAGNNLKLFTDRLHNQHGDILAGNSLWVQKDASGGANTEIINNSGNIETHQGDIVVRTGHLLNQREGFSATTTTRTNPSSIQGMGNALVDIPLSLLPDGSYGYFTREVENQHGTPCNGHGACNITMDTLYYYAPFADSATQRFLSSQNITTVTGADNPAGRIASGRNLSAEAERLENRASFILANGDIALSGRELSNQSWQTGTENEYLVYRYDPKTFYGSYATGSLDKLPLLSPEFENNTIRFSLDGREKDYTPGKTYYSVIQAGGDVKTRFTSSINNGTTTAHAGSVSPVVSAPVLNTLSQQTGGDSLTQTALQQYEPVVVGSPQWHDELAGALKNIAGGSPLTGQTGISDDWPLPSGNNGYLVPSTDPDSPYLITVNPKLDGLGQVDSHLFAGLYELLGAKPGQAPRETAPSYTDEKQFLGSSYFLDRLGLKPEKDYRFLGDAVFDTRYVSNAVLSRTGSRYLNGLGSDTEQMRYLMDNAARQQKGLGLEFGVALTAEQIAQLDGSILWWESVTINGQTVMVPKLYLSPEDITLHNGSVISGNNVQLAGGNITNSGGSINAQNDLSLDSSGYIDNLNAGLISAGGSLDLSAIGDISNISSVISGKTVQLESVSGNISNITRRQQWNAGSDSQYGGVHLSGTDTGPVATIKGTDSLSLDAGKNIDITGATVSSGGDLGMSAGNDINIAANLISGSKSQSGFWHTDDNSSSSTTSQGSSISAGGNLAMAAGHNLDVTASSVSAGHSALLSCRSRPSLECSQGKAKTSRNGRSESHESHAAVSTVTAGDNFLLVAGRDIASQAAGMAAENNVVIRGGRDVNLVAESAGAGDSYTSKKKKEINETVRQQGTEIASGGDTTVNAGRDITAVASSVTATGNISVNAGRDVALTTATESDYHYLETKKKSGGFLSKKTTRTISEDSATREAGSLLSGNRVTVNAGDNLTVEGSDVVADRDVSLAAGNHVDVLAATSTDTSWRFKETKKSGLMGTGGIGFTIGSSKTTHDRREAGTTQSQSASTIGSTAGNVSITAGKQAHISGSDVIANRDISITGDSVVVDPGHDRRTVDEKFEQKKSGLTVALSGTVGSAINNAVTSAQETKESSDSRLKALQATKTALSGVQAGQAATMASATGDPNAGVSLSLTTQKSKSQQHSESDTVSGSTLNAGNNLSVVATGKNRGDNRGDIVIAGSQLKAGGNTSLDAANDILLSGAANTQKTTGRNSSSGGGVGVSIGAGKGAGISAFASVNAAKGREKGNGTTTDKTVTINSGRDTVLNGAQVNGNRIIADVGHDLLISSQQDTSKYDSKQTSVAAGGSFTFGSMTGSGYIAASRDKMKSRFDSVAEQTGMFARVMVASTSQWVNIPNWMVRSLPHCHTGEKPPGYRTLGLVTLQRSGIIKSSHRWNQS

SEQ ID NO: 44 - protein, 321 aa, Escherichia coli
MMLKKTIFILTLFSGNVIAATVELGFENEQYNYAYRSADVFMPYIKSNFNPVTDSALNVSLTYMYQDQYGKKHKKTSEDRFKTNRDRIELYLKGYTLNRGAYSFSPSAGFRYESWDVNYDNPKKQDKWKLELRFYPNMTYKLNDQLSLYMNGFVAPVFFKTQQESRKDNNYVKGKLGAKRYNNDYYQELQILGVRYKFNNDNTLWASVYNERKYNQHSSKYDRWQLRGGYDFKVTEEFVLSPFIRYDLSYREKNLESTSNNGLSKNNKEIRTGASFSYKIIPSVKLVGEIYRQTTNIENYYGEHSEDKNRMFYKLGINKTF

SEQ ID NO: 45 - protein, 587 aa, Escherichia coli
MQHRQKNILTKTSLLSRALSVPCCDMFRRGSPWICYLSLSVFSGCFIPAFSSPAAMLSPGDRSAIQQQQQQLLDENQRQRDALERPLTITPSPETSAGTEGPCFTVSSIVVSGATRLTSAETDRLVPWVNQCLNITGLTAVTDAVTDGYIRRGYITSRAFLTEQDLSGGVLHITVMEGRLQQIRAEGADLPARTLKMVFPGMEGKVLNLRDIEQGMEQINRLRTEPVQIEISPGDREGWSVVTLTALPEWPVTGSVGIDNSGQKSTGTGQLNGVLSFNNPLGLADNWFVSGGRSSDFSVSHDARNFAAGVSLPYGYTLVDYTYSWSDYLSTIDNRGWRWRSTGDLQTHRLGLSHVLFRNGDMKTALTGGLQHRIIHNYLDDVLLQGSSRKLTSFSVGLNHTHKFLGGVGTLNPVFTRGMPWFGAESDHGKRGDLPVNQFRKWSVSASFQRPVTDRVWWLTSAYAQWSPDRLHGVEQLSLGGESSVRGFKDQYISGNNGGYLRNELSWSLFSLPYVGTVRAVAALDGGWLHSDSDDPYSSGTLWGAAAGLSTTSGHVSGSFTAGLPLVYPDWLAPDHLTVYWRVAVAF

SEQ ID NO: 46 - protein, 744 aa, Escherichia coli
MNKHTLLLTVLFLNLICTPVFAQNWQVATFGQSTDLNFSSLIDSAKIGRNNAWLAGNNNFLEAGKFYTLPTDFFIESRGGKIANSHDGMTVFYTIVPVTQTFRLEADLTLEQIGPEVNGKSPAGQEGAGLFVRDIIGPQRQEPQSAGTEEYPQASNILMNAFITQNKKNDNLVQITSIVREGVIKTWGNEGITIKKQPIIENINFTQKRNIHMTIERLPEKFILTAFDTDRKENQSWQFSDYSGFMNQLDNNSLAIGFFAARNAKLRVKNASFKPGKPLVDYKQLTSRQFSRVRHKAPELFLASPQSVVRNSTTLQFLANQAGIVSIDNDKQTKQVQAGELVQFPVTLQKKHNDFTVNFNVDGNISKKAIRIEQVKSNLTDPYEIYVCSDCRQGARGSKNDPVDLQTAVKFVAPGGNIYLNDGQYHGITLDRELSGIPGKYKTISAINPHKAIFINKTFNLDASYWHLKSVVFDGNVDNGNNKPAYLRIAGSYNIIEHVIARNNDDTGISISAKDKNRFFWPAHNLVLNSDSYNNLDLSGINADGFAAKLGVGPGNIFRGCIAHNNADDGWDLFNKIEDGPNASVTIENSVAYENGLPYNKADILKGSIGNGGEGQPSKSQVINSIAINNNMDGFTDNFNTGSLIVRNNIAMNNARYNYILRTNPYKFPSSILFDNNYSIRDDWENKIKDFLGDTVNSVNYKLLVSHETGPVQKDLFFTRDDSGNIIYPDFFLNIINKFNZTMP

SEQ ID NO: 47 - protein, 136 aa, Escherichia coli
MKTFIKTLLVAVTILFSVFATAKQVKLPNNIKYVNTTEAFSCTEIDGMNCQTKNPFNYKDNSYVFVLERGGAWCYDYTVSVLNLKTGKAQMLEYKDNQLCSGSNKPFFEIKNGVPTVGVIDTSGKPVVVALDKLKT

SEQ ID NO: 48 - protein, 225 aa, Escherichia coli
MQLPVKLLMSLISLVSVIARAGKYKNYIRDEIKYWRYTSYKGGEFPEGFTDEKFSSAIYNGRIFTMKRLHTLMLFLAVLFTGFNVEAASVKQALSCDPNARAEQPGACPTTYELYEGDAAYKAALDKALKPVGLSGMFGKGGYMDGPGGNVTPVTINGTVWLQGDGCKANTCGWDFIVTLYNPKTHEVVGYRYFGLDDPAYLVWFGEIGVHEFAYLVKNYVAAVN

SEQ ID NO: 49 - protein, 721 aa, Escherichia coli
MKTQITFAALLPALASFIPLHAHASSTSEDEMIVTGNTAADTTDSAAGAGFKTNDIDVGPLGTKSWIETPYSSTTVTKEMIENQQAQSVSEMLKYSPSTQMQARGGMDVGRPQSRGMQGSVVANSRLDGLNIVSTTAFPVEMLERMDVLNSLTGALYGPASPAGQFNFVAKRPTEETLRKVTLGYQSRSAFTGHADLGGHFDENKRFGYRVNLLDQEGEGNVDDSTLRRKLVSVALDWNIQPGTQLQLDASHYEFIQKGYVGSFNYGPNVKLPSAPNPKDKNLALSTAGNDLTTDTISTRLIHYFNDDWSMNAGVGWQQADRAMRSVSSKILNNQGDISRSMKDSTAAGRFRVLSNTAGLNGHIDTGSIGHDLSLSTTGYVWSLYSAKGTGSSYSWGTTNMYHPDAIDEQGDGKIRTGGPRYRSSVNTQQSVTLGDTVTFTPQWSAMFYLSQSWLQTKNYDKHGNQTNQVDENGLSPNAALMYKITPNTMAYVSYADSLEQGGTAPTDESVKNAGQTLNPYRSKQYEVGLKSDIGEMNLGAALFRLERPFAYLDTDNVYKEQGNQVNNGLELTAAGNVWQGLNIYSGVTFLDPKLKDTANASTSNKQVVGVPKVQANLLAEYSLPSIPEWVYSANVHYTGKRAANDTNTSYASSYTTWDLGTRYTTKVSNVPTTFRVVVNNVFDKHYWASIFPSGTDGDNGSPSAFIGGGREVRASVTFDF

SEQ ID NO: 50 - protein, 669 aa, Escherichia coli
MKNITLWQRLRQVSISTSLRCAFLMGALLTLIVSSVSLYSWHEQSSQIRYSLDKYFPRIHSAFLIEGNLNLVVDQLNEFLQAPNTTVRLQLRTQIIQHLDTIERLSRGLSSRERQQLTVILQDSRSLLSELDRALYNMFLLREKVSELSARIDWLHDDFTTELNSLVQDFTWQQGTLLDQIASRQGDTAQYLKRSREVQNEQQQVYTLARIENQIVDDLRDRLNELKSGRDDDIQVETHLRYFENLKKTADENIRMLDDWPGTITLRQTIDELLDMGIVKNKMPDTMREYVAAQKALEDASRTREATQGRFRTLLEAQLGSTHQQMQMFNQRMEQIVHVSGGLILVATALALLLAWVFNHYFIRSRLVKRFTLLNQAVVQIGLGGTETTIPVYGNDELGRIAGLLRHTLGQLNVQKQQLEQEITDRKVIEADLRATQDELIQTAKLAVVGQTMTTLAHEINQPLNALSMYLFTARRAIEQTQKEQASMMLGKAEGVISRIDAIIRSLRQFTRRAELETSLHAVDLAQMFSAAWELLAMRHRSLQATLVLPQGTATVSGDEVRTQQVLVNVLANALDVCGQGAVITVNWQMQGKTLNVFIGDNGPGWPEALLPSLLKPFTTSKEVGLGIGLSICVSLMEQMKGELRLASTMTRNACVVLQFRLTDVEDAK

SEQ ID NO: 51 - protein, 753 aa, Escherichia coli
MNVIKLAIGSGILLLSCGAYSQSISEKTNSDKKGAAEFSPLSVSVGKTTSEQEALEKTGATSSRTTDKNLQSLDATVRSMPGTYTQIDPGQGAISVNIRGMSGFGRVNTMVDGITQSFYGTSTSGTTTHGSTNNMAGVLIDPNLLVAVDVTRGDSSGSEGINALAGSANMRTIGVDDVIFNGNTYGLRSRFSVGSNGLGRSGMIALGGKSDAFTDTGSIGVMAAVSGSSVYSNFSNGSGINSKEFGYDKYMKQNPKSQLYKMDIRPDEFNSFELSARTYENKFTRRDITSDDYYIKYHYTPFSELIDFNVTASTSRGNQKYRDGSLYTFYKTSAQNRSDALDINNTSRFTVADNDLEFMLGSKLMRTRYDRTIHSAAGDPKANQESIENNPFAPSGQQDISALYTGLKVTRGIWEADFNLNYTRNRITGYKPACDSRVICVPQGSYDIDDKEGGFNPSVQLSAQVTPWLQPFIGYSKSMRAPNIQEMFFSNSGGASMNPFLKPERAETWQAGFNIDTRDLLVEQDALRFKALAYRSRIQNYIYSESYLVCSGGRKCSLPEVIGNGWEGISDEYSDNMYIYVNSASDVIAKGFELEMDYDAGFAFGRLSFSQQQTDQPTSIASTHFGAGDITELPRKYMTLDTGVRFFDNALTLGTIIKYTGKARRLSPDFEQDEHTGAIIKQDLPQIPTIIDLYGTYEYNRNLTLKLSVQNLMNRDYSEALNKLNMMPGLGDETHPANSARGRTWIFGGDIRF

SEQ ID NO: 52 - protein, 133 aa, Escherichia coli
MSSKTKCWLWMLLVILSETSATSTLKMFDNSEGMTKTLLLALIVVLYCICYYSLSRAVKDIPVGLAYATWSGTGILMVSTLGILFYGQHPDTAAIIGMVIIASGIIIMNLFSKMGSEEAEETPVTNLDKKIAN

SEQ ID NO: 53 - protein, 286 aa, Escherichia coli
MYIKKHWIALSILLIPCIGNAQEIKIDESWLHQSLNVIGRTDSRFGPRLTNDLYPEYTVAGRKDWFDFYGYVDLPKFFGVGSHYDVGIWDEGSPLFTEIEPRFSIDKLTGLNLAFGPFKEWFIANNYVYDMGDNQSSRQSTWYMGLGTDIDTGLPIKLSANIYAKYQWQNYGAANENEWDGYRFKIKYSIPLTNLFGGRLVYNSFTNFDFGSDLADKSHNNKRTSNAIASSHILSLLYEHWKFAFTLRYFHNGGQWNAGEKVNFGDGPFELKNTGWGTYTTIGYQF

SEQ ID NO: 54 - protein, 172 aa, Escherichia coli
MRIAPRTFFAISALAFIVASGFSFWRLSPAENTGIMSCSTKGIMRFENMEKENVNGNIHFNFGSQGKGSMVLEGYTDSAAGWLYLQRYVKFTYTSKRVSATERHYRISQWESSASSIDESPDVIFDYFMREMSDSHDGLFLNAQKLNDKAILLSSINSPLWICTLKSGSKLD

SEQ ID NO: 55 - protein, 182 aa, Escherichia coli
MKIKVIALATFVSAVFAGSAMAYDGTITFTGKVVAQTCTVNTSDKDLAVTLPTVATSSLKDNAATSGLTPFAIRLTGCATGMNSAQNVKAYFEPSSNIDLATHNLKNTATPTKADNVQIQLLNSNGTSTILLGEADNGQDVQSETIGSDGSATLRYMAQYYATGQSTAGDVKATVHYTIAYE

SEQ ID NO: 56 - protein, 359 aa, Escherichia coli
MKRIFFIPLFLILLPKLAVAGPDDYVPSQIAVNTSTLPGVVIGPADAHTYPRVIGELAGTSNQYVFNGGAIALMRGKFTPALPKIGSITVYFPSRKQRDSSDFDIYDIGVSGLGIIIGMAGYWPATPLVPINSSGIYIDPVGANTNPNTYNGATASFGARLFVAFVATGRLPNGYITIPTRQLGTILLEAKRTSLNNKGLTAPVMLNGGRIQVQSQTCTMGQKNYVVPLNTVYQSQFTSLYKEIQGGKIDIHLQCPDGIDVYATLTDASQPVNRTDILTLSSESTAKGFGIRLYKDSDVTAISYGEDSPVKGNGSQWHFSDYRGEVNPHINLRANYIKIADATTPGSVKAIATITFSYQ

SEQ ID NO: 57 - protein, 844 aa, Escherichia coli
MNANNLSCLIYCRCSLLLFAALGLTVTNHSFAAEEAEFDSEFLHLDKGINAIDIRRFSHGNPVPEGRYYSDIYVNNVWKGKADLQYLRTANTGAPTLCLTPELLSLIDLVKDTMSGNTSCFPASTGLSSARINFDLSTLRLNIEIPQALLNTRPRGYISPAQWQSGVPAAFINYDANYYQYSSSGTSNEQTYLGLKAGFNLWGWALRHRGSESWNNSYPAGYQNIETSIMHDLAPLRAQFTLGDFYTNGELMDSLSLRGVRLASDERMLPGSLRGYAPAVRGIANSNAKVTIYQNAHILYETTVPAGPFVINDLYPSGYAGDLLVKITESNGQTRMFTVPFAAVAQLIRPGFSRWQMSVGKYRYANKTYNDLIAQGTYQYGLTNDITLNSGLTTASGYTAGLAGLAFNTPLGAIASDITLSRTAFRYSGVTRKGYSLHSSYSINIPASNTNITLAAYRYSSKDFYHLKDALSANHNAFIDDVSVKSTAFYRPRNQFQISINQELGEKWGGMYLTGTTYNYWGHKGSRNEYQIGYSNFWKQLGYQIGLSQSRDNEQQRRDDRFYINFTLPLGGSVQSPVFSTVLNYSKEEKNSIQTSISGTGGEDNQFSYGISGNSQENGPSGYAMNGGYRSPYVNITTTVGHDTQNNNQRSFGASGAVVAHPYGVTLSNDLSDTFAIIHAEGAQGAVINNASGSRLDFWGNGVVPYVTPYEKNQISIDPSNLDLNVELSATEQEIIPRANSATLVKFDTKTGRSLLFDIRMSTGNPPPMASEVLDEHGQLAGYVAQAGKVFTRGLPEKGHLSVVWGPDNKDRCSFVYHVAHNKDDMQSQLVPVLCIQHPNQEKT

SEQ ID NO: 58 - protein, 277 aa, Escherichia coli
MVKCHTLINRRNKCLLIVFIVLIGWIIFRPKAYTYSLNDKEKEMLIMLSQHPETRYFGFYSIELPADYKPTGMVMFIQGSAMIPVETKLQYYPPFLQYMTRYEAELKNTSALDPLDTPYLKQVHPLSPPMNGVIFERMKAKYTPDFARVLDAWKWENGVTFSVKIEAKDGRATRYDGISKIAEYSYGYNIPEKKVQLLTILSGLQPRADNQPPSENKLAIQYAQVDASLLGEYELSVDYKNSNNIKISLQTDNNSYIDSLLDIRYPSNGNRAWYNSI

SEQ ID NO: 59 - protein, 366 aa, Escherichia coli
MLPEPVYRRWIILLISMLTVGTLFILSVWNSATYWDIFIYGVLPMLFLWLCLFGIALNKYEQSVAACISWESERQQVKQLWQHWSQKQLAIVGNVLFTPEEKGMSVLLGPQEEIPAYPKKARPLFSASRYSLSSIFHDIHQQLTQQFPDYRHYLHTIYVLQPEKWRGETVRQAIFHQWDLVPERTNTLNQIQSLYDERFDGLILVVCLQNWPENKPEDTSELVSAQLISSSSFVRQHQIPVIAGLGRVMPLEPEELEHNLDVLFEYNQLDNKQLQHVWVSGLDEGTIENLMQYAEQHQWSLPKKRPLHMIDHSFGPTGEFIFPVSLAMLSEAAKETEQNHLIIYQSAQYAQKKSLCLITRKLYLRT

SEQ ID NO: 60 - protein, 260 aa, Escherichia coli
MLNRKLNIRLRHSLNSHCIPSIIINNTVRSFQRSVMNTRALFPLLFTVASFSASAGNWAVKNGWCQTMTEDGQALVMLKNGTIGITGLMQGCPNGVQTLLGSRISINGNLIPTSQMCNQQTGFRAVEVEIGQAPEMVKKAVHSIAERDVSVLQAFGVRMEFTRGDMLKVCPKFVTSLAGFSPKQTTTINKDSVLQAARQAYAREYDEETTETADFGSYEVKGNKVEFEVFNPEDRAYDKVTVTVGADGNATGASVEFIGK

SEQ ID NO: 61 - protein, 385 aa, Escherichia coli
VVIINSTILSGAGAIPSLTSLLPDIRKMLLVTDRNIAQLDGVQQIRALLEKHCPQVNVIDNVPAEPTHHDVRQLMDAPGDASFDVVVGIGGGSVLDVAKLLSVLCHPQSPGLDALLAGEKPTQRVQSWLIPTTAGTGSEATPNAILAIPEQSTKVGIISQVLLPDYVALFPELTTSMPAHIAASTGIDALCHLLECFTATVANPVSDNAALTGLSKLFRHIQPAVNDPQDLRAKLEMLWASYYGGVAITHAGTHLVHALSYPLGGKYHLPHGVANAILLAPCMAFVRPWAVEKFARVWDCIPDAETALSAEEKSHALVTWLQALVNQLKLPNNLAALGVPPEDIASLSEAALNVKRLMNNVPCQIDLQDVQAIYQTLFPQHPFKE

SEQ ID NO: 62 - protein, 105 aa, Escherichia coli
MNIRKLFCPGNTPRILLFLFFFVVSAITTIACGYTEKNATGNVLLLFLLLLLAHRNTLTSITALLFLFCCALYAPAGMTYGKINNSFIVALLQTTTDEAAEFTGM

SEQ ID NO: 63 - protein, 147 aa, Escherichia coli
MNIQAIKEMVNLICSFLFIFFLSSAFVSFGCYAIYELFLWNDIIVYSWGYILIVFLPFTLYVMSFEILFFAISGRRLSKVTMVRLWLIIKIIIAFSICAVLIFSSIYKKELLSRNYIACSGIPSGWMPGLATKYVKEKSLCEKNGNN

SEQ ID NO: 64 - protein, 178 aa, Escherichia coli; identical to SEQ ID NO: 37

SEQ ID NO: 65 - protein, 209 aa, Escherichia coli
MFLKRKWYYAVTTSVVITLCGGGYYMYRQEYQMVVTVPTADANDPNWPNKRIQFDTSEWLQQLQYIKIDDHYILNTQYTPIANLDDFGITLKLQNALNGSDKRLPALYGLAEMDAQKFKDLMRGKIKCEYLRTTFDAETLKPVNDYFLISFTYKDKWYEFETERKISKTSDDGYFLWAFDNTVHEAGYWHNTDPAAYSYRDYQNGKAVK

SEQ ID NO: 66 - protein, 424 aa, Escherichia coli
MDIWRGHSFLMTISARFRQYVFSLMSILLQERKMNIFTLSKAPLYLLISLFLPTMAMAIDPPERELSRFALKTNYLQSPDEGVYELAFDNASKKVFAAVTDRVNREANKGYLYSFNSDSLKVENKYTMPYRAFSLAINQDKHQLYIGHTQSASLRISMFDTPTGKLVRTSDRLSFKAANAADSRFEHFRHMVYSQDSDTLFVSYSNMLKTAEGMKPLHKLLMLDGTTLALKGEVKDAYKGTAYGLTMDEKTQKIYVGGRDYINEIDAKNQTLLRTIPLKDPRPQITSVQNLAVDSASDRAFVVVFDHDDRSGTKDGLYIFDLRDGKQLGYVHTGAGANAVKYNPKYNELYVTNFTSGTISVVDATKYSITREFNMPVYPNQMVLSDDMDTLYIGIKEGFNRDWDPDVFVEGAKERILSIDLKKS

SEQ ID NO: 67 - DNA, 489 bp, Escherichia coli
atgaaactgaaagctattatattggccaccggtcttattaactgtattgtattttcagcacaggcagtggatacgacgattactgtgacgggtaatgttttgcaaagaacatgtaatgtaccagggaatgtggatgtttctttgggtaatctgtatgtatcagactttcccaatgcaggaagtggatctccatgggttaattttgatctgtctctcaccggatgccagaatatgaatactgttcgggcaacatttagtggtactgcggatgggcagacatactatgcgaatacagggaatgctggcggtatcaagattgaaattcaggacagggatggaagtaatgcatcatatcacaatggtatgttcaagacgcttaatgtacaaaataataatgcaacctttaatcttaaagcccgtgcagtgagtaaaggccaggttactcctggaaatatcagttctgttataaccgtcacctatacctatgcg

SEQ ID NO: 68 - DNA, 2019 bp, Escherichia coli
atgaaaatgacacggctttatcctctggccttggggggattattgctccccgccattgctaatgcccagacttcacagcaagacgaaagcacgctggtggttaccgccagtaaacaatcttcccgctcggcatcagccaacaacgtctcgtctactgttgtcagcgcgccggaattaagcgacgccggcgtcaccgccagcgacaaactccccagagtcttgcccgggctcaatattgaaaatagcggcaacatgcttttttcgacgatctcgctacgcggcgtctcttcagcgcaggacttctataaccccgccgtcaccctgtatgtcgatggcgtccctcagctttccaccaacaccatccaggcgcttaccgatgtgcaaagcgtggagttgctgcgaggcccacagggaacgttatatggcaaaagcgctcagggcgggatcatcaacatcgtcacccagcagccggacagcacgccgcgcggctatattgaaggcggcgtcagtagccgcgacagttatcgaagtaagttcaacctgagcggccccattcaggatggcctgctgtacggcagcgtcaccctgttacgccaggttgatgacggcgacatgattaaccccgcgacgggaagcgatgacttaggcggcacccgcgccagcatagggaatgtgaaactgcgtctggcgccggacgatcagccctgggaaatgggctttgccgcctcacgcgaatgtacccgcgccacccaggacgcctatgtgggatggaatgatattaagggccgtaagctgtcgatcagcgatggttcaccagacccgtacatgcggcgctgcactgacagccagaccctgagtgggaaatacaccaccgatgactgggttttcaacctgatcagcgcctggcagcagcagcattattcgcgcaccttcccttccggttcgttaatcgtcaatatgtctcagcgctggaatcaggatgtgcaggagctgcgcgctgcaaccctgggcgatgcgcgtaccgttgatatggtgtttgggctgtaccggcagaacacccgcgagaagttaaattcagcctacgacatgccgacaatgccttatttaagcagtaccggctataccaccgctgaaacgctggccgcatacagtgacctgacctggcatttaaccgatcgttttgatatcggcggcggcgtgcgcttctcgcatgataaatccagtacacaatatcacggcagcatgctcggcaacccgtttggcgaccagggtaagagcaatgacgatcaggtgctcgggcagctatccgcaggctatatgctgaccgatgactggagagtgtatacccgtgtagcccagggatataaaccttccgggtacaacatcgtgcctactgcgggtcttgatgccaaaccgttcgtcgccgagaaatccatcaactatgaacttggcacccgctacgaaaccgctgacgtcacgctgcaagccgcgacgttttatacccacaccaaagacatgcagctttactctggcccggtcgggatgcagacattaagcaatgcgggtaaagccgacgccaccggcgttgagcttgaagcgaagtggcggtttgcgccaggctggtcatgggatatcaatggcaacgtgatccgttccgaattcaccaatgacagtgagttgtatcacggtaaccgggtgccgttcgtaccacgttatggcgcgggaagcagcgtgaacggcgtgattgatacgcgctatggcgcactgatgccccgactggcggttaatctggtcgggccgcattatttcgatggcgacaaccagttgcggcaaggcacctatgccaccctggacagcagcctgggctggcaggcgactgaacggatgaacatttccgtctatgtcgataacctgttcgaccgtcgttaccgtacctatggctacatgaacggcagcagcgccgtcgcgcaggtcaatatgggtcgcaccgtcggtatcaatacgcgaattgatttcttc

SEQ ID NO: 69 - DNA, 738 bp, Escherichia coli
atgaataaggtttttgttgtttcagtggtggccgcagcctgtgtatttgcagtaaatgcaggagcaaaggaaggtaaaagcggtttttatctgaccggtaaagccggtgcctctgtgatgtcactttcagaccagcgtttcctgtcaggagatgaggaagaaacatcaaagtataaaggcggcgatgaccatgatacggtattcagtggcggtattgcggtcggttatgatttttatccgcagttcagtattccggttcgtacagaactggagttttacgctcgtggaaaagctgattcgaagtataacgtagataaagacagctggtcaggtggttactggcgtgatgacctgaagaatgaggtgtcagtcaacacactaatgctgaatgcgtactatgacttccggaatgacagcgcattcacaccatgggtatccgcagggattggctacgccagaattcaccagaaaacaaccggtatcagtacctgggattatgagtacggaagcagtggtcgcgaatcgttgtcacgttcaggctctgctgacaacttcgcatggagccttggcgcgggtgtccgctatgacgtaaccccggatatcgctctggacctcagctatcgctatcttgatgcaggtgacagcagtgtgagttacaaggacgagtggggcgataaatataagtcagaagttgatgttaaaagtcatgacatcatgcttggtatgacttataacttc

SEQ ID NO: 70 - DNA, 498 bp, Escherichia coli
atgaaactgaaagctattatattggccaccggtcttattaactgtattgcattttcagcacaggcagtggatacgacgattactgttacagggagggtattgccacgtacctgtaccattggtaatggaggaaacccaaacgccaccgttgttttggataacgcttacacttctgacctgatagcagccaacagcacctctcagtggaaaaatttttcgttgacattgacgaattgtcagaatgtaaacaatgttacttcatttggtggaaccgcagaaaatacaaattattacagaaatacaggggatgctactaatatcatggttgagctacaggaacaaggtaatggtaatacccccttgaaagttggttcaacaaaagttgttacagtgagcaatgggcaggcgacattcaatcttaaagtccgtgccgtaagcaaaggtaatgctggtgcgggaagtattaattcacaaattactgtcacctatacctatgcg

SEQ ID NO: 71 - DNA, 3885 bp, Escherichia coli
atgaataaaatatactcccttaaatatagtgctgccactggcggactcattgctgtttctgaattagcgaaaagagtttctggtaaaacaaaccgaaaacttgtagcaacaatgttgtctctggctgttgccggtacagtaaatgcagcaaatattgatatatcaaatgtatgggcgagagactatcttgatcttgcacaaaataaaggtattttccagcccggagcaacagacgtaacaatcactttaaaaaacggagataaattctctttccataatctctcaattccggatttttctggtgcagcagcgagtggcgcagctaccgcaataggaggttcttatagtgttactgttgcacataacaaaaagaaccctcaggccgcagaaacccaggtttacgctcagtcttcttacagggttgttgacagaagaaattccaatgattttgagattcagaggttaaataaatttgttgtggaaacagtaggtgccaccccggcagagaccaaccctacaacatattctgatgcattagaacgctacggtatagtcacttctgacggttcaaaaaaaatcataggttttcgtgctggctctggaggaacatcatttattaatggtgaatccaaaatctcaacaaattcagcatatagccatgatctgttaagtgctagtctatttgaggtcacccaatgggactcatacggcatgatgatttataaaaatgataaaacatttcgtaatcttgaaatattcggagacagcggctctggagcatacttatatgataacaaactagaaaaatgggtattagtcggaacaacccatggtattgccagcgttaatggtgaccaactgacatggataacaaaatacaatgataaactggttagtgagttaaaagatacctatagtcataaaataaatctgaatggcaataatgtaaccattaaaaacacagatataacattacaccaaaacaatgcagataccactggtactcaagaaaaaataactaaagacaaagatattgtgttcacaaatgggggagatgtcctgtttaaggataatttggattttggtagcggtggtattatctttgacgaaggccatgaatataacataaacggtcagggatttacatttaaaggagcaggaattgatatcggaaaagaaagcattgtaaactggaatgcattgtattccagtgatgatgttttacacaaaataggccccggtactctgaatgttcaaaaaaaacagggggcaaatataaagataggtgaaggaaatgttattcttaatgaagaaggaacatttaacaatatataccttgcaagcggaaatggtaaggtaatactaaataaagataattcccttggcaatgatcaatatgcggggatattttttactaaacgtggtggtacgctagatttaaatggacacaatcagacttttactagaattgccgccactgacgatggaacaacaataactaactcagatacaacgaaagaagccgttctggcaatcaataacgaagactcctacatatatcatgggaacataaatggcaatataaaactaacgcacaatattaattctcaggataagaaaactaatgcaaaattaattctggatggtagtgtcaacacaaaaaatgatgttgaagtcagtaatgccagtcttaccatgcaaggccatgcaacagagcatgcaatattcagaagctcagcgaatcattgctccctggtatttctttgtggaacggactgggtcaccgttttgaaagaaacagagagttcatataataaaaaattcaattctgattacaaaagtaataatcagcagacctcatttgatcagcctgactggaaaaccggggtgtttaaatttgatacattacacctgaacaatgctgacttttcaatatcacgcaatgccaatgttgaaggaaatatatcagcaaataaatcagctatcacaatcggcgataaaaatgtttacattgataatcttgcagggaaaaatattactaataatggttttgacttcaaacaaactatcagtactaatctatccataggagaaactaaatttacaggtggcatcactgcacataacagccaaatagccataggtgatcaagctgtagttacacttaatggtgcaacctttctggataatactcctataagtatagataaaggagcaaaagttatagcacaaaattccatgttcacaacaaaaggtattgatatctccggtgaactgactatgatgggaatccctgaacagaatagtaaaactgtaacgccgggtctccactacgctgctgatggattcaggctgagtggtggaaatgcaaatttcattgccagaaatatggcatctgtcaccggaaatatttatgctgatgatgcagcaaccattactctgggacagcctgaaactgaaacaccgactatatcgtctgcttatcaggcatgggcagagactcttttgtatggctttgataccgcttatcgaggcgcaataacagcccccaaagctacagttagcatgaataatgcgatctggcatctaaatagccagtcatcaattaatcgtctagaaacaaaagacagtatggtgcgttttactggtgataatgggaagtttacaacccttacagtgaacaaccttactatagatgacagtgcatttgtgctgcgtgcaaatctggcccaagcagatcagcttgttgtcaataaatcgttgtctggtaaaaacaaccttctgttagtcgacttcattgagaaaaatggaaacagcaacggactgaatatcgatctggtcagcgcaccaaaaggaactgcagtagatgtctttaaagctacgactcggagtattggcttcagtgatgtaacaccggttatcgagcaaaagaacgatacagacaaagcaacatggactctgatcggctataaatctgtggccaacgccgatgcggctaaaaaggcaacattactgatgtcaggcggctataaagccttccttgctgaggtcaacaaccttaacaaacgtatgggtgatctgcgtgacattaacggtgagtccggtgcatgggcccgaatcattagcggaaccgggtctgccggcggtggattcagtgacaactacacccacgttcaggtcggtgcggataacaaacatgaactcgatggccttgacctcttcaccggggtgaccatgacctataccgacagccatgcaggcagtgatgccttcagtggtgaaacgaagtctgtgggtgccggtctctatgcctctgccatgtttgagtccggagcatatatcgacctcatcggtaagtacgttcaccatgacaacgagtataccgcaactttcgccggccttggcaccagagactacagctcccactcctggtatgccggtgcggaagtcggttaccgttaccatgtaactgactctgcatggattgagccgcaggcggaacttgtttacggtgctgtatccgggaaacagttctcctggaaggaccagggaatgaacctcaccatgaaggataaggactttaatccgctgattgggcgtaccggtgttgatgtgggtaaatccttctccggtaaggactggaaagtcacagcccgcgccggccttggctaccagtttgacctgtttgccaacggtgaaaccgtactgcgtgatgcgtccggtgagaaacgtatcaaaggtgaaaaagacggtcgtatgctcatgaatgttggtctcaacgccgaaattcgcgataatcttcgcttcggtcttgagtttgagaaatcggcatttggtaaatacaacgtggataacgcgatcaacgccaacttccgttactctttc

SEQ ID NO: 72 - DNA, 426 bp, Escherichia coli
atgattaatattcccagtcccaccgctgttgttatggcgctggtagccatcagcacgcttcccagccctagcagggtaaagcttatgccatatcctcccagagcccacaacaccacaggtttactgccagtacgggaaatttgctttccccaccacggggacgatggcagaaacagcattgagccaagcatcagcagggcagcccatacagacagactcagatttgtctgtatgaccagaacagggagcacaaccagcagaccgttctgcccgataccgagaagcccggcactgaacgcaagtggccagcaggacagtggtttttggggcgtatcttcgatcccaggtgacattttaatgtttcaactccatgtattaattgtgtttatttgtaaaattaatttatctgacaataacatttcttat

SEQ ID NO: 73 - DNA, 954 bp, Escherichia coli
atgtatgcccgcgagtatcgctcaacacgcccgcataaagcgattttctttcatctttcttgcctcacccttatctgtagtgcgcaagtttatgcgaagccggatatgcggccactggggccgaatatagccgataaaggctccgtgttttaccatttcagcgccacctctttcgactctgtcgatggcacacgccattatcgggtatggacggccgtgccgaatacaaccgcaccggcatcgggttacccgattttatatatgcttgacggtaacgcagttatggaccgcctggatgacgaactgctcaaacaattgtcagaaaaaacaccgccagtgatcgtggctgtcgggtatcagaccaacctccctttcgatctcaacagcagggcttacgactatacgccagcagcagaaagcagaaaaacagatctccactcagggcgttttagccgtaagagtggtggcagcaacaacttccgccagttactggaaacgcgtattgccccaaaagtggaacagggactgaatatcgatcggcaacgccgcggcttatgggggcactcctacggcggcctcttcgtgctggattcctggctgtcctcctcttacttccggtcgtactacagcgccagcccgtcgttgggcagaggttatgatgctttgctaagccgcgttacggcggttgagcctctgcaattctgcaccaaacacctggcgataatggaaggctcggcgacacagggtgataaccgggaaacgcatgctgtcggggtgctgtcgaaaattcataccaccctcactatactgaaagataaaggcgtcaatgccgtattttgggatttccccaacctgggacacgggccgatgttcaatgcctcctttcgccaggcactgttagatatcagtggtgaaaacgcaaattacacagcaggttgtcatgagttaagccac

SEQ ID NO: 74 - DNA, 2175 bp, Escherichia coli
atgagaattaacaaaatcctctggtcgctaactgtgctcctagttgggttgaatagccaggtatcagtagccaaatactccgacgatgataatgacgagactctggtggtggaagccaccgctgagcaggtattaaaacagcagccgggcgtgtcggttattaccagcgaggatattaaaaagacccctccggtaaacgacctttcagatattattcgtaaaatgcctggtgttaatcttaccggcaatagcgcctcgggcacacgcggtaataaccgccagatcgatattcgtggtatggggccggaaaacaccttaattttaattgatggtgtaccggtgacgtcacgtaactccgtgcgttatagctggcgtggggagcgtgatacccgcggtgacaccaactgggtgccaccggaacaggttgagcgtattgaagtgatccgcggccctgcggcggcgcgctacggttcgggggccgccgggggggtggtgaacatcattaccaaacgtcccaccaacgactggcacggttcgctgtcgttatacaccaaccagccggaaagtagcgaagagggcgctacgcgtcgcgccaatttcagccttagtgggcctctggctggtgatgctcttaccacgcgtttgtatggtaacctgaataaaacggatgctgacagttgggatattaattctccggtcggtacgaaaaacgcagccgggcatgaaggggtacgtaacaaagatattaacggcgttgtctcgtggaaattaaatccgcagcagattctcgatttcgaagtcggatatagccgccaggggaatatctatgcgggcgatacgcagaacagttcttccagtgcagttaccgaaagcctggcaaaatccggcaaagagacgaaccgcctgtaccgacagaattatggcattacgcataatggtatctgggactggggacaaagtcgctttggtgtttattacgagaaaaccaataatacccgcatgaatgaaggattatccggcggtggtgaaggacgtattttagcgggtgaaaagtttacgaccaatcgcctgagttcctggcgaaccagcggtgagcttaatattcctttgaatgtgatggttgatcaaacgctgaccgttggtgcagagtggaaccgcgataagctcgatgatccttcctctaccagcctgacggtgaatgacagagatatcagcggtatttctggctctgctgcggatcgcagcagtaaaaatcattctcaaatcagtgcgctgtatattgaagataacattgagccggttcctggcacgaatatcattcccggcctgcgctttgattatctcagcgactccggcgggaacttcagccccagtctgaatctttcgcaggaattgggcgattatttcaaagtcaaagcaggggttgcccgaacctttaaagccccaaacctgtatcaatccagtgaaggctatctgctctactcgaaaggcaatggctgtccaaaagatattacatcaggcgggtgctacctgatcggtaataaagatctcgatccggaaatcagcgtcaataaagaaattggactggagttcacctgggaagattaccacgcaagtgtgacctacttccgcaatgattaccagaataagatcgtggccggggataacgttatcgggcaaaccgcttcaggcgcatatatcctcaagtggcagaatggcgggaaagctctggtggacggtatcgaagccagtatgtctttcccactggtgaaagagcgtctgaactggaataccaatgccacatggatgatcacttcggagcaaaaagacaccggtaatcctctgtcggtcatcccgaaatatactatcaataactcgcttaactggaccatcacccaggcgttttctgccagcttcaactggacgttatatggcagacaaaaaccgcgtactcatgcggaaacccgcagtgaagatactggcggtctgtcaggtaaagagctgggcgcttattcactggtggggacgaacttcaattacgatattaataaaaatctgcgtcttaatgtcggcgtcagtaatatcctcaataaacagatcttccgatcttctgaaggggcgaatacctataacgagccaggccgggcttattatgccggagttaccgcatcattc

SEQ ID NO: 75 - DNA, 3042 bp, Escherichia coli
atgggtaaccaatggcaacaaaaatatcttcttgagtacaatgagttggtatcaaatttcccttcacctgaaagagttgtcagcgattacattaagaattgttttaaaactgacttgccgtggtttagtcggattgatcctgataatgcttatttcatctgcttttctcaaaaccggagtaatagcagatcttatactggatgggatcatcttgggaaatataaaacagaagtactgacactcactcaagccgctcttattaatattggttatcgttttgatgtttttgatgatgcaaattcaagcacaggaatttataaaacaaagagtgcagatgtgtttaacgaagaaaatgaagaaaaaatgctcccgtcggaatacctgcattttttacaaaagtgtgattttgcaggtgtttatggaaaaactctgtcagattactggtcgaaatactatgataaatttaagcttttactaaaaaattattatatttcttctgctttgtatctttataaaaatggagagcttgatgagcgtgaatataatttctccatgaacgccttaaatcgcagtgataatatatcactattattctttgatatttatggatattacgcatctgatatttttgtagccaaaaataatgataaggtaatgcttttcattcctggtgcaaaaaaaccttttttattcaagaagaatatcgctgatttgcggcttacccttaaagaacttattaaggatagtgacaacaaacaattactttcccaacatttttcattatatagtcgtcaagatggagtttcctatgcaggagtaaattctgttctacatgcaatagaaaatgatggtaattttaatgagtcttactttctgtattccaataagacacttagcaataaagatgtttttgatgctatagctatttctgttaagaaacgcagtttcagtgatggtgatatcgttataaaatcaaacagtgaagctcaacgagactatgctctgactatactccagacgattttatcaatgacccctatatttgatatcgtagtcccggaggtatctgttccgcttggactggggattattacttccagtatggggatcagttttgatcaactgattaatggtgatacttatgaagaacgtcgttctgctatacctggtttggcgacaaatgcagtattgcttggtctgtcttttgcaattccactcttgattagtaaggcaggaataaaccaggaggtacttagcagcgttataaataatgagggcaggactctgaatgaaacaaatatcgatatatttttgaaggaatatggaattgctgaagatagtatatcctcaactaatttgttagacgttaagcttaaaagttccgggcagcatgtcaatattgtaaagcttagtgatgaagataatcaaattgtcgctgtaaaagggagttctctgagcggcatctactatgaagtggacattgaaacaggatatgagattttatcccgaagaatttatcgtaccgaatataataatgaaattctctggactcgaggtggtggtctaaaaggggggcagccatttgattttgaaagtctcaatattcctgtattttttaaagatgaaccctattctgcagtgaccggatctccgttatcatttattaatgatgacagctcacttttatatcctgatacaaacccaaaattaccgcaaccaacgtcagaaatggatattgttaattatgttaagggttctggaagctttggggatagatttgtaactttgatgagaggagctactgaggaagaagcatggaatattgcctcttatcatacggctgggggaagtacagaagaattacacgaaattttgttaggtcagggcccacagtcaagcttaggttttactgaatatacctcaaatgttaacagtgcagatgcagcaagcagacgacactttctggtagttataaaagtgcacgtaaaatatatcaccaataataatgtttcatatgttaatcattgggcaattcctgatgaagccccggttgaagtactggctgtggttgacaggagatttaattttcctgagccatcaacgcctcctgatatatcaaccatacgtaaattgttatctctacgatattttaaagaaagtatcgaaagcacctccaaatctaactttcagaaattaagtcgcggtaatattgatgtgcttaaaggacggggaagtatttcatcgacacgtcagcgtgcaatctatccgtattttgaagccgctaatgctgatgagcaacaacctctctttttctacatcaaaaaagatcgctttgataaccatggctatgatcagtatttctatgataatacagtggggctaaatggtattccaacattgaacacctatactggggaaattccatcagactcatcttcactcggctcaacttattggaagaagtataatcttactaatgaaacaagcataattcgtgtgtcaaattctgctcgtggggcgaatggtattaaaatagcacttgaggaagtccaggagggtaaaccagtaatcattacaagcggaaatctaagtggttgtacgacaattgttgcccgaaaagaaggatatatttataaggtacatactggtacaacaaaatctttggctggatttaccagtactaccggggtgaaaaaagcagttgaagtacttgagctacttacaaaagaaccaatacctcgcgtggagggaataatgagcaatgatttcttagtcgattatctgtcggaaaattttgaagattcattaataacttactcatcatctgaaaaaaaaccagatagtcaaatcactattattcgtgataatgtttctgttttcccttacttccttgataatatacctgaacatggctttggtacatcggcgactgtactggtgagagtggacggcaatgttgtcgtaaggtctctgtctgagagttattctctgaatgcagatgcctccgaaatatcggtattgaaggtattttcaaaaaaattt

SEQ ID NO: 76 - DNA, 1362 bp, Escherichia coli
atggtggacatgattaatgaaagtgcacggcaaacgccagtcattgcacaaacggacgttctggttatcgggggcggtccggcaggattatccgctgccattgcggcagggcggttaggtgccagaaccatgattgttgagcgctacgggtcgctaggcggcgtattgacgcaggtcggggtagaaagttttgcctggtatcgtcatccggggacggaagattgtgaagggatctgtcgtgagtatgaaggccgcgcacgagcgctgggtttcacacgaccagaacctcagtcaattagcgaagttatagatactgaaggatttaaagttgtcgccgatcagatgattacggaatctggcgttgagccgttatatcactcctgggttgtggacgtgatcaaggacggggatacgttatgcggtgttatcgtcgagaataaatcaggtcgaggggcaattctggcgaaaagaatcgtcgattgcacgggggatgctgatattgccgctcgtgcaggcgcgccctggacgaaacggagcaaggaccaactgatgggcgtcaccgtgatgttcagttgcgcaggtgttgatgtggcacgctttaaccgttttgttgcggaagaacttaagccgacctacgcggattggggcaaaaactggacgattcaaaccacgggtaaagaagacccgatgtttagcccgtatatggaggatatttttacccgcgcgcaacaggatggtgtgattccaggtgacgcccaggcgattgccggaacctggtcgaccttttctgaaagcggtgaggctttccagatgaatatggtgtacgcctttggttttgactgtaccgatgtcttcgatttaaccaaagctgagattgccggaaggcagcaagcattatgggcaattgacgcactacgccactatgttccgggctttgaaaatgtacggttacgcaattttggtgccacgctggggacgcgtgaatcacggcttattgagggggaaatacgtattgctgatgattacgtccttaatcaggggcgttgttcggacagtgtagggattttcccggaatttattgatggttccggttatctcattttgccaacgaccgggcgtttctttcagatcccttatggttgtctggtgccgcaaaaagtggagaaccttttggtcgccggtcgctgtatttccgcaggcgtagttgcacatacttctatgcgtaacatgatgtgttgtgccgttaccggtgaggccgcaggtactgccgccgtggtttcgctacagcaaaattgcaccgtgcgtcaggttgctatccctgatttgcaaaacacgctgcaacagcagggcgttcgtctggca

SEQ ID NO: 77 - DNA, 759 bp, Escherichia coli
atgtctgccaaaagacgacttcttattgcgtgtaccttgataacagctatctatcattttcctgcatattcttcattagaatataaaggaacctttggttcaataaatgcgggttatgcagactggaacagtggatttgtaaacactcaccgtggtgaagtatggaaagtgactgcggattttggggtaaattttaaagaagcagaattttactcattttatgaaagtaatgtactcaatcatgctgtagcagggagaaatcatacggtttcagcaatgacgcatgtcagactctttgactctgatatgacattctttggcaaaatttatggccaatgggataactcatggggtgacgatctggacatgttttatggattcggttacctcggctggaacggcgagtggggcttttttaaaccgtatattggattgcataatcaatctggtgactacgtatcagctaaatatggtcaaacgaatggttggaatggttatgttgttggctggacagcagtattaccatttacgttatttgacgaaaaatttgttttatctaactggaatgaaatagaactggacaggaacgatgcttacacggagcagcaatttggccggaacgggttaaatggcggtttaactattgcctggaagttctatcctcgctggaaagcaagtgtgacgtggcgttatttcgataataagctgggctacgatggctttggcgatcaaatgatttatatgcttggttatgatttc

SEQ ID NO: 78 - DNA, 1476 bp, Escherichia coli
atggccagtttgatcggccttgcagtttgcacagggaatgcttttagtcctgccttagccgcagaggctaaacaacctaatttagtcattattatggcggatgatttaggttatggcgatttagcaacatatggtcatcagatcgttaaaacacctaatatcgacaggcttgcccaggaaggggtcaaatttactgactactatgcccccgctcctttaagttcaccttcacgcgcagggctattaaccggccggatgccatttcgtactggaattcgctcatggattccttcaggcaaagatgttgccttagggcgtaacgaactcacgattgctaatctactcaaagcgcaagggtacgacacggcaatgatgggtaagctgcatctgaatgcaggcggcgatcgcaccgatcagccacaagcacaagatatgggctttgattactcactggctaatacggcgggctttgttaccgacgccacgctggataacgctaaagaacgcccgcgttatggcatggtttacccgacaggctggctacgtaatgggcaacccactccacgagccgataaaatgagcggtgagtatgtcagttcggaagtcgtcaactggctggataacaaaaaggacagcaagcctttcttcctctatgttgcttttaccgaagtgcatagccccctggcttcgcccaaaaaatacctcgacatgtactcacaatatatgagcgcgtatcagaagcagcatcctgatttattttatggcgactgggcagacaaaccctggcgtggtgtgggggaatattatgccaatatcagctatctggatgcacaggttggaaaagtgctggataaaatcaaagcgatgggtgaagaagataacacaatcgttatttttaccagtgataacggtccggtaacgcgtgaagcgcgcaaagtgtatgagctgaatttggcaggggaaacggatggattacgcggtcgcaaggataacctttgggaaggcggaattcgtgttccagccattattaaatatggtaaacatctaccacagggaatggtttcagatacacccgtttatggtctggactggatgcctactttagcgaaaatgatgaacttcaaattacctacagaccgtactttcgatggtgaatcgctggttcctgttcttgagcaaaaagcattgaaacgcgaaaagccattaattttcgggattgatatgccattccaggatgatccaaccgatgaatgggcgatccgtgatggtgactggaagatgattatcgatcgcaataataaaccgaaatatctctacaatctgaaatctgatcgttatgaaacacttaatctgatcggtaaaaaaccagatattgaaaaacagatgtatggtaagtttttaaaatataaaactgatattgataatgattctctaatgaaagccagaggtgataaaccagaagcggtgacctggggc

SEQ ID NO: 79 - DNA, 954 bp, Escherichia coli
gtgacaacaactatctgcgctatgggcgaattgctggccgagtttttgtcccgcaacccacatcaaaaattcactcagcctggggagtttatcgggccatttcccagcggtgcgccagcaatttttgctgctcaggtggcaaaactgtcccatcgggccatcttctttggatgtgttggtaatgatgattttgcccgactcattatagagcgtctccgtcatgaaggtgtcattaccgatgggatccatgttatgaacaatgccgtcacaggtacggcgttcgtgagttatcaaaatccccagcagcgggatttcgtctttaatatccctaacagcgcctgcggtttgtttactgccgagcacattgataaggatctgcttaaacagtgtaaccatctgcatattgtgggctcatcgttgttctcatttcgcatgatcgatgtcatgcgtaaagcaataacgacgatcaaatcggctggcggcaccgtttctttcgatcccaatattcgcaaagagatgctgagcattcctgaaatggcgcaggctctcgattatttgattgaatatacggatatttttatccccagcgaaagcgaactccctttcttcgcgcgtcacaaaaatctgtcagaggaacagattgttagcgatcttctccacggcggcgtaaaacatgtggcgataaaacgcgcccagcgtggggccagctattacaagcttaaaaacggtacattacacgcccagcatgttgcaggtcacgatatcgaaattatcgatccaacgggtgcaggcgactgctttggcgcaacgtttatcactcttttcttatccggtttcccggcacacaaggcgctgcaatatgcaaatgccagcggcgcgctcgccgtaatgcggcaaggtccgatggaagggatatcctcactggcagacattgaagactttttgcagcagcac

SEQ ID NO: 80 - DNA, 513 bp, Escherichia coli
atgaagatattcattagtttatttttgtttataatatcaacaaattcttttgctgatgatatcactcatgccggagtggttcgtattgaagggttaattaccgaaaaaacctgcattatttctgatgagtcaaaaaattttacagttaatatgccagacgtacccagtagttcggtaaggagtgcaggggatgttactgaaaaggtttatttttccataacgttaacccgctgtggtagtgatgttggcaacgcgtatataaagtttaccggcaatacagtttctgaagatgccagtttatataagctggaagatggctcggtagaggggcttgcacttacgatttttgataagaacaaaggcagtattagtaatgatgttaaaagcatggttttttcacttacatcatcagttgataatatattgcatttttttgcggcttacaaagcattaaaaaataatgtccaaccaggggatgcaaatgcgtcagtatcgtttattgtcacctatgat

SEQ ID NO: 81 - DNA, 603 bp, Escherichia coli
atgattaaattccggctttatattccccctgtaattctcggttttgttatcgtaccattattggtatggccgacggttattgccttagccgtacttatattcacgttaacttttctggcggaaataatattctcctttccgctcctggttgtgcgtatttctcttcaggaattacaacttgagttattggttgtatatgcactttttttcagtgtaatgggtggcatcggttggcaattctcccgcagaacgcctcctgaattaaaaaacaggctacattgctggctggtcttttctccggtctatttctggttaattctctcgaatttcattctttatatttctccagagaaatcagcgttgctggaaaatatccgaaatttctttctgacatttgtctggcttcccctgaatttttcccctttttggccgcagccgtggactgattttgtcggcccgattagtgcccagcttggttttgcgttgggatattattgccagtggcgtagcaaaaatagaagccataggaagaagtggggcgattgggtaacgtgcttaagtttggcgattttagctctggggccgttattcaattatttacaa

SEQ ID NO: 82 - DNA, 702 bp, Escherichia coli
atgaaattcaatttatctaatttatccgcagtattactggcatcaggtatgctgatgtctactgcggtaaccgcagcacccggcgatgcaacacaatttggtggggcggatactgactggagcaccgttgattatcccaggctcactgatatggatgacaacgttgattcaatgggggggaaaatccgctttactggccgtgtagtgaaagctacctgtaaggtcgcaaccgattcaaaacagattgaagttgtcctgccggttgtgccttccaaccttttcactggtatcgacgtagaagcacagggggcgagcaaccagaccgatttcaatattaatctgaccgaatgtagcaatacagatgatcagaaaattgagttccgttttaccggtactgcagatagcgctaataaaacgctcgctaacgaagtagaaggatcaacggatgctgacaacagcggcaatgcgggggcgactggtgtagggattcgaatttactccaaaggtacgacgaataatggtctgattaacctgaataccactgcggcagagggtagcgcctccaccgccgcttatacaattccaggaaatgctacgacccatgatttcagcgcggcctttactgcaggttatgctcaaaacggtagcactgttgcaccaggtgtagttaagtcaacagcaagttttgttgtgctgtacgag

SEQ ID NO: 83 - DNA, 1008 bp, Escherichia coli
atgcgtatacatacttattggtatagaagatatttcattttattgattattatattttcaaatgttctttcttctattgctaatgctgaagatatggggcgagaacgtgcatattgttatccgggttcaccgagtaataatactacgcctgcatccttttcttataattttggtactatagtggtttctgatgtcaacaaaaatgcgcctggcactgtattgccatcacaaatctggaaggttggaacctataaggcttattgtaattctcttgatgattatgaaatttacttcagtgctgtctctggaatagatccgtctggtgccagtggtgatcatcaagggagtgatgtatttattccactcacccatgaaatatctgtctctactcatataaaactttataatcaaaatggcacaatgacagataaaattgtgccattcgaaaattataataccaattatccgggggacagaagcaaaccatctaattgggcatcaggtactgaaggatatattaaaatcaggattgataaaaaaattatatctgatgtttcattaagtaacgtattattggtgtcattatatgtcagccagatccctaccgaacatggtcctatccctgtctttaatgcctacataggaaacttaaatattcaggttccgcaaggttgcactattaatgagggtacgagttttactgttaatatgccggatgtgtgggccagtgaattgagccgggctggtgccggagcgaagcccgctggtgttactcctgtagcaacaactattccgattaattgtacgaataaagatacagatgcggtaatgacgttggtattcgacggtaacatttccgccacacgtgataccaatgggaaacaaagtattattcaggcacaagataatcctgatgttggtattatgattatggatagtcagcaaaactccgtagatttaaatgccctggcaacatcagtaggcgttccgttcagattggtggaaaac

SEQ ID NO: 84 - DNA, 2592 bp, Escherichia coli
atgaacctaaagctcaaaagatgcgaatattggatggcggcacaaaagcagatgaaacgggttgtgccgcttcttctggttattatgcctgcatgttcaatcgcgggaatgcgctttaaccctgcttttctgtcgggtgatactgaagctgttgctgacttatcccgcttcgagaaagggatgacttatcttcctggtagctatgaagtcgaagtttgggtcaatgattcccctttactctctcgtactgtaacttttaaagcagacgatgagaatcaactgattccctgcctttcacttgctgacttattaagccttggaattaacaaaaatgcgctgccagagcaggctttggcttcatctgaaaatagttgccttgatttgcgtatctggtttcccgatgtgcattacatgccggagctggatgcacagagacttaaactgacctttccacaggcgataataaaacgtgacgctcgcggatatattccaccagaacagtgggataacggtattacagcttttttgctgaattatgacttttctggtaataacgatcgtggtgattactcttcaaataactattatttaaatcttcgcgctgggatcaatattggtgcatggcgttttcgcgattattcaacctggagtcgtgggagtaattcagcaggtaaactggagcatatcagtagtacgttgcagcgcgttattattcctttcagaagtgaattaacgctaggagatacatggtcatcatcagatgttttcgacagtgttagtattcgtggcataaaactggaatctgacgaaaatatgttgcccgatagtcaaagtggtttcgctcccacggtgcgcggaattgcgaaaagtcgcgctcaggtaacaatcaaacagaatggttatgtcatttatcaaacctatatgccgccgggaccgtttgagattagcgatcttaacccgacatcatctgcgggagatctggaagttaccatcaaagagtctgataattcagaaactgtctataccgtaccttatgccgctgtccccatcctgcaacgagaaggtcatttaaaatattctactacggttggccaatatcgaagcaatagctataaccagaaaagtccttatgtatttcagggggaattaatttggggtttaccctgggatattacggcttatggtggggcacaattctctgaggattaccgggcgttggcgctcggccttggcctgaatctgggtgtatttggtgcaacatcgtttgatgttactcaggctaacagttcgcttgtggatgggagcaaacatcaagggcaatcttatcgttttctttattccaaatcgttagttcagacaggaacagcattccatattattggctatcgttattcaacccagggcttttacactttaagtgatacgacataccaacaaatgtcagggactgttgttgatccaaaaacgttagatgataaagattacgtttataactggaatgatttttataacttgcgttatagcaaacgtggaaaatttcaggctagtgtatcgcaacctttcggtaactacgggtctatgtatttatcggctagtcagcaaacatactggaatactgataaaaaagattctttataccaagttggttataacaccagtattaagggtatctatctaaatgttgcgtggaattacagtaaatcaccagggacaaatgcggataaaattgtctcgctaaatgtctcattacctataagtaattggttatcttccacgaatgatgggcgctcatcatcgaatgccatgactgcaacgtatggttatagtcaggataaccacggacaggtaaaccaatatacgggggtatctggttctctgttggagcagcataatctcagttataacatacaacatggttttgctaatcaggataatagcagtagtggttctgttggtgttaattatcgtggggcatatggttccttgaattccgcctacagttacgataatgaaggtaatcaacaaataaactatggcatcagtggtgctcttgttgtacatgaaaatggtcttacgttgagtcaaccattaggtgaaactaatgttttgataaaagcgcctggagcgaataatgtggatgttcagcgggggacaggaatatccactgactggcgtggatatgcagttgttccttatgcaacagaatatagacgtaataatatttcattagatcctatgtcaatgaatatgcatactgaactggatatcacttccactgaagttattccgggaaaaggtgcgttagttcgtgcagagtttgctgctcatatcggtattcgtggtttgttcacagttcgttatcgtaataaatcagtcccattcggtgctacagccagcgctcagattaaaaacagtagtcaaattaccgggattgtcggcgataatggacaactttatctctcaggattgcctttagaaggtgttattaatatccagtggggagacggtgttcagcaaaaatgtcaggctaattacaagctccctgaaacagaactggataatcctgttagctatgcaactctggagtgccgc

SEQ ID NO: 85 - DNA, 507 bp, Escherichia coli
atgggagcgatttatgttaaacgtttgattctgtcggtagcactgataataccgatagcatccaatgcttctgatgctttgaaccagccgagcagtagtctaaatgatggtgttgagactttttttatttcctgctttgatatgcctcaggaaacaactactgatatggacgcttgtcagagagttcagttagctcaggttagttgggttaagaataagtattcggtggccgccctgaatcgtttgaaacaagacaacaaggatgatccacagcgtctgcaggaattaactgcttcttttaacgcggaaagtgaagcttggacagaattaattgagaaagcgtcaaagtccgtccaggttgattatgtaggaggaactatagctggcactgcagttgcatcacgtcaaattggtcttctggaattacaatcccacgatatctgggagcactggctacgatctcgaggactcaactcctcctcttttgccagaaccaaagttcaaatc

SEQ ID NO: 86 - DNA, 2139 bp, Escherichia coli
atggctatgttcacaccttcattctcaggactcaaaggtcgggcgctcttttcactgctttttgcggcaccgatgattcatgcaacagactctgtaacgaccaaagatggcgaaacaatcactgttacagcagatgcaaataccgcaactgaggcaaccgatggttatcaacctctgagcacctccacggcgacattaaccgatatgccgatgctggatatcccgcaggtggtcaatacggttagcgatcaggttctggaaaaccagaatgcgacaacgctggatgaggcgctttataacgtcagtaacgtggtacagaccaatacattaggcgggactcaggatgcttttgtacgccgtgggtttggcgcaaaccgggatggctccatcatgaccaacggtctgcgaaccgtacttcctcgtagtttcaacgccgcaacagagcgtgtggaagtgctaaaaggcccggcctccacgctgtatggcattctcgatcctggcggactgattaacgtcgtgaccaagcgcccggaaaaaacattccatggttcggtttcagccacctcctccagttttggtggcggcactgggcaacttgatatcacaggtcccattgaaggcactcagctggcgtatcgccttaccggggaagtgcaggatgaagattactggcgaaacttcggtaaagagcgcagtacatttattgccccgtcactcacctggtttggtgataatgcaacagtaaccatgctctattcccatcgggactataaaactccattcgatcgtggaacgattttcgaccttacgacgaaacagcccgtaaacgttgatcgaaaaatacgttttgacgaaccgtttaatattacagatggtcagtccgatctggcgcaactcaacgcagaatatcatctcaatagccagtggacagcgcgctttgattacagctacagccaggataaatacagcgataatcaggcgcgtgttaccgcgtatgatgcaacgacaggaacactgacacggcgtgttgatgcaactcagggatctacccagcgtatgcatgctactcgtgcggatctgcaagggaatgttgatattgccggattctataatgagattctgggtggggtgtcatatgaatattatgatcttctgcgtacagatatgattcgctgtaaaaaagctaaagatttcaatatatacaaccctgtttatggtaataccagcaaatgtacaacggtttcggcgtcggacagcgatcagacgatcaaacaggagaactactcagcttatgcacaggacgcgctctatctgaccgataactggattgccgtcgccgggatccgctatcagtattacacgcaatatgcgggtaaaggccgtccttttaatgtcaatactgacagccgcgatgaacaatggacgcccaaactggggttagtctacaaactgacgccatcggtatccttatttgccaattattcgcaaacatttatgccgcagtcgtcaattgccagctacattggcgatcttccaccagaatcatctaatgcttacgaagtcggggcaaaattcgagctattcgatggtatcaccgcagatattgcgctgtttgatatccataaacgtaatgtgttgtataccgaaagtattggtgatgaaaccatcgccaaaacggcaggccgcgttcgttcaagaggggtagaagtcgaccttgcgggagcattaactgaaaacattaatatcattgccagctacggctataccgatgcaaaggttctggaagatcctgattatgcagggaaaccattgccgaatgttcctcgtcataccggttcgctattcctgacctatgatattcataacatgccaggcaataacacactgacgtttggcggtggcggacatggtgtaagccgtcgttcggcaaccaatggggctgactattatctgcctggctatttcgttgccgatgccttcgccgcatacaaaatgaaattgcagtatccggtcactctgcaattaaacgtcaaaaacctgtttgataaaacgtattacacctcttccatcgccacaaataatctgggcaaccagattggcgatccgcgtgaagtgcaattcacggtgaaaatggaattt

SEQ ID NO: 87 - DNA, 1818 bp, Escherichia coli
atgaaaatatcgtggaattatatatttaagaacaaatggcgatttcacattacaagcatttcactttttcttatcatgctcgcggtttcaatcgcttttttgcacttgcgttttaataccttgtccagtaccgataaaatgcggcttgaaatgtataagtccacattatattccaccatcgagcaattttatgttttaccctatatgctctcaacagaccatatcatccgtcaggcggtaattacgcctgacgatatgacgtccagcgaactcaatcaacgaattgcacatttcaatactcaactcaaaaccgcagcaatatttattctggatacccaaggtaaggccatcgcttctagcaactggcaggaccccggcagctatgtagggcaaaattatagctatcgcccctattataaacacgccatgtctggcttaaatggacgcttttacggtattggtagcactacgaatacaccgggattcttcctctctacaagtataaaagataaaggaaaaattgtcggtgttgtagtagtaaaaataagtcttaatgaaattgaaaaagcatgggccgaaggtcctgaaaatattatcgtgaatgatgaacatgggattatatttttaagttcaaaatcgccatggcgaatgcgaacactgcaaccgttacctgttcaggcaaaacaaaaactacaatctacccgccaatatagtctcgacaatcttttaccggcggattattatccctgttataccgtgagcaattttactttcctgaaagataaaaaagaacaactctgtttattcccgcaatattatacgcaacaaatagccattccagaatttaactggaaaatgacaattatggtccccttagataacctgtactggtcatgggctatttcgttagtcattacactaattatttacctgctgtttttgttatttattaaatactggagaatgcgatctcatgcacaacaattattaacacttgcgaatgaaacattagaaaaacaggttaaagagcgtacatctgccctggaattgatcaatcaaaaattaatacaggagataaaagagcgcagtcaagctgaacaagtattacaaattacgcgtagtgaactggcagagtccagcaaactggcggcgcttggacagatggcaaccgaaattgcccatgaacaaaatcaaccgttagccgccattcacgcacttactgataacgcgcgtactatgctaaaaaaagagatgtatccgcaggttgaacagaatctgaaacatattatttcagtgattgagcggatgacgcagctcatttccgaacttaaagcatttgcctcgcgccatcgcgtacctaaaggttctgccgatgtcatcaaagtgatgtatagcgccgtggcgttacttaatcacagcatggagaaaaataacattgagcgacgaataaaagccccatccatgccgttatttgtcaattgcgatgagctcggtcttgaacagatattcagtaatttaattagcaacgccttagattctatggaaggtagctcttacaaacgactggatatcgccattcgccaggcaaataacaaagttattattaccattaaagacagcggtggcggttttgcacctgaagttgtcgatcgcatatttgaaccattttttaccactaaacgtagaggaatggggttgggactggcaatagtcagcgaaattgtccgaaattcgaacggcgcactccacgccagtaatcatcctgaaggcggcgcagtaatgacattaacctggcctgaatggggagaagaacatgaa

SEQ ID NO: 88 - DNA, 303 bp, Escherichia coli
gtgcttacaccacaacatttacgttgtgtgttaacatgtagcgatttactgactcttttgagtggtaccgttatgtctcaaatgcccctctattttcttaatacccaaaagaaactcactgctcactatgaatggcttcaaatcaacctgactgatacctacgaactagttaaaaggttaatgccgattccttcactggacgtggtggttaaagtagggaaacttgtcctcccggagaaagggcatcatggtttttaccctgaagctggagttgtctatagaacagtagctccagaaaatcca

SEQ ID NO: 89 - DNA, 789 bp, Escherichia coli
atgatgaaaaatacaggctatatcttagctctttgtctgacagcatcggggcatgtcctagcccatgatgtctggattacaggtaaacaggcagagaacaacgttaccgcagagattggttatggtcataatttcccctcaaaggggacaattcctgacagaagggatttctttgaaaatccccggctttataacgggaaagagacaataacactgaagccagcgtccacggattatgtctataaaactgagtctgcaagcaaagataatggttacgttctgtcaacgtatatgaaaccgggatactggtcgagaacctcgtcaggatggaaaccggtcagccgggagggcagaaatgatgtggcttactgtgaatttgtcactaaatatgcaaaatcttttattcctggtgaacagcagatgccagcacaactctatcagtctccaacagggcatgagcttgaaatcattccgttatccgatataagtcgtttcagtgaaaatgtgaagctgaaagttctgtataaaacgtccccgctcgccggagctatcatggagcttgactcggtcagttatctgacatcatcccgtcatactcatgcagttgagcacaaacatcctgttcataaagcagaactcacctttgtaactaatgaggatggtatcgtcacagtaccttctcttcatatcggacagtggctggcgaaagtccaaaataagaaaagttttcaggacaaaagcctgtgtgatgaaactgtcgatgtggcaaccttaagcttctcccgaaat

SEQ ID NO: 90 - DNA, 1134 bp, Escherichia coli
atgggaaaaataaaatattggctaatagtaggatttattatactttttgcgattttttacattgctattagtgacagggattctacgctttctaggttgaaatcagcaggtgaaaacggagatgtagaagctcagtatgctttggggctcatgtatttgtatggagaaattctggatgttgattatcagcaggcaaagatttggtatgaaaaagccgctgaccaaaatgatccgcgtgcgcaggccaaactcggtgtgatgtatgcaaatggtctcggggtaaatcaggattatcagcaatcaaaattatggtatgaaaaggcggctgcgcaaaatgatgttgatgcgcaatttttgcttggggagatgtatgacgatggtctcggggtaagccaagactaccagcatgcaaagatgtggtatgaaaaagcggctgctcaaaatgatgagcgtgctcaggtcaatctcgctgttctatacgcaaagggtaatggtgttgaacaggattatcgacaggccaaaagctggtatgaaaaggctgcagctcaaaatagtcctgatgcgcagttcgctcttggaattctgtatgccaatgctaatggtgtagagcaggactatcagcaggcaaaagactggtatgagaaagcagcagaacaaaatttcgccaatgctcagtttaatcttggtatgctctattacaaaggtgagggtgttaaacaaaactttcggcaagccagagaatggtttgaaaaagccgcatctcaaaatcagccgaatgcccaatataatttaggtcagatttattactacggtcagggtgtgactcagagctatcgacaggcgaaagactggtttgaaaaagcggcagagaaaggtcatgtcgatgctcaatataatctcggtgtaatatacgaaaatggtgaaggtgtgagtcagaactatcaacaggcaaaggcttggtatgaaaaggcagcctcacaaaatgatgcgcaggcgcagttcgaacttggcgttatgaatgaactgggtcagggtgaaagcatagacctgaaacaagcaagacattactatgagcggtcatgtaataatgggcttaagaaaggttgtgaacggttaaaagagttattatacaaa

SEQ ID NO: 91 - DNA, 1962 bp, Escherichia coli
atgaatgtaatcagaactgtcatttgtacattaattatacttccggtgggattacaggcagcgaccagtcattcttctatggttaaagatacaatcaccattgtcgcgacaggaaatcagaacacggtatttgaaacgccgtcgatggtcagtgtcgtcacgaatgacacaccgtggagtcagaatgcggttacatcggccggcatgctgaaaggtgttgccggtctcagccagactggtgcaggacggaccaatgggcagacctttaatttacgcggctatgacaaaagcggggtacttgttcttgttgacggcgttcgccaactcagtgacatggcaaaaagcagtggcacttatctggatccggcactcgtcaaacgtatcgaagttgtccgcgggccaaactccagtctgtacggcagtggcgggctgggaggtgtagtggacttcagaactgccgatgcagcagattttcttccccccggagagacaaacggtttaagtctgtggggaaatatcgccagtggtgaccacagcacaggctcggggctcacctggtttggtaaaactggaaaaacagatgcgctcctttctgtcattatgcgtaaaagaggtaatatctatcaaagtgatggtgagcacgcacctaacaaggaaaaacctgcagccctgtttgcgaaaggctctgtcggtataacagacagtaacaaagcaggtgccagcttgcgtctctaccggaataacaccactgaaccgggcaattccactcagacacatggtgacagcggcctgcgtgacagaaaaacagtacaaaatgacgtacagttctggtaccagtacgctcctgtggataacagcctcatcaatgtaaagtcaacgttatatctcagtgatatcactatcaagacaaacggtcacaacaaaacggcagaatggagaaacaacagaacctccggtgttaatgttgtcaacaggagtcatactctgatttttccgggagcccatcagttaagttatggcgctgaatattaccgtcagcagcagaagccagaaggctctgccacactatatccggaaggaaacattgactttacatcgttgtatttccaggatgaaatgacaatgaaaagctacccggttaacattatcgtcggttcccgctatgaccggtacaagagcttcaatccccgtgccggagaactgaaagccgaacgcctgtccccaagggcggcgatttcagtctcaccgacagactggctgatgatgtacggctccatatcctctgcattccgagcgcccacaatggcagaaatgtacagggatgatgtacatttttaccgcaagggtaaacccaattactgggttcctaaccttaatctgaaaccagaaaataacatcacccgtgagattggcgcaggtattcaactggatggcctgcttacagacaatgaccggctgcagttaaaaggcggatatttcggaacggatgccagaaactatattgccacacgcgtggatatgaaacggatgcgttcttattcttataatgtatcccgggcccgtatctggggatgggatatgcagggtaattaccagtctgattatgttgactggatgctttcttataaccggacggaaagtatggatgccagcagcagggaatggctgggctccggcaatcctgacacacttatcagtgacatcagcatacctgttggtcatagaggcgtttatgccggatggcgtgctgaactttcagcatcagccacgcatgtgaaaaaaggcgatccccatcaggctggttataccatacattccttttcactgtcttataagcctgtaagtgttaaaggctttgaggcgtcagtaactctggataatgccttcaacaagcttgccatgaatggcaaaggtgtgccgctttcaggcagaactgtcagtctttatacccgttatcagtgg

SEQ ID NO: 92 - DNA, 4128 bp, Escherichia coli
atgaataaaatatacgctctaaaatattgttatattactaacacagtaaaggttgtctctgaactagcccgaagggtatgtaaagggagtacccgcagaggaaaaagactttcagtacttacctctctggcactatctgcattactcccaaccgttgctggtgcatcaacggttggtggcaacaatccttaccagacataccgcgactttgcagaaaacaaagggcagtttcaggctggcgcaacaaacattcctatttttaataataaaggggaattagtaggacatcttgataaagcgcccatggttgattttagcagtgtgaatgtaagctcaaatcccggcgttgcaacattaattaacccgcaatatatagccagtgtaaaacataataaaggatatcagagcgtcagcttcggtgatggtcagaacagttaccatattgtggatcgtaatgaacacagttcatctgatctccacacaccaagacttgataagctcgtaactgaggttgctccggctaccgtaaccagctcatcaacagctgatatattgaacccttcaaaatactcggcattctacagggctggttcgggaagtcagtatattcaggatagtcagggtaagcgacattgggtaacaggtgggtatggttatctgacaggaggaatactcccgacatcattcttttatcacggctcagacggcattcagctgtatatggggggcaacatacatgatcatagcatcctgccctcttttggagaggccggcgacagtggttctccattatttggctggaatacggccaaagggcagtgggaactggtcggtgtttactcgggagtaggaggggggaccaatttgatatattctcttattcctcagagttttctctcacagatctattcagaggataatgacgctcccgtcttttttaatgcctcatccggcgcccccctgcaatggaaatttgacagcagcaccggcactggctctctgaaacagggttccgatgaatatgccatgcacgggcaaaaaggttctgacctgaacgcaggtaaaaatctgacattcctgggacataatggtcagattgacctggaaaactctgtcacgcagggtgccggttcactgacatttactgatgactacactgtcaccacttcaaacggaagtacctggaccggggccggtattattgtggacaaggatgcctccgtaaactggcaggttaatggtgtgaaaggtgacaacctgcataaaatcggcgaaggaaccctggttgtacagggaaccggtgttaatgagggcggcctgaaagtcggggatgggaccgttgtcctcaatcagcaggctgacagttcaggacacgttcaggcattcagtagcgtgaatattgccagcggccgcccgacagtcgtgctggcagacaaccagcaggttaatccggacaatatatcctggggctaccgggggggggttctggatgttaacgggaatgacctgacatttcataagctgaatgccgccgattatggcgcaactctcggtaacagcagtgataaaacggctaatatcactctggattatcagacgcgtccggcagacgtaaaagttaatgaatggtcatcatcaaacaggggaacagtaggttcattatatatttataataatccctatactcataccgtcgattattttatcctgaaaacaagtagttatggctggttccctaccggtcaggtcagtaacgagcactgggaatatgtcggacatgaccagaacagtgcacaggcactgcttgcaaacagaattaataataaagggtatctgtatcatggcaagttgctgggaaatattaatttctcaaataaagcaaccccgggtacaaccggcgcattggttatggacggctcagcgaatatgtccggtacatttactcaggaaaacggtcgtctgaccattcagggccacccggttatccatgcttcaacgtctcagagtattgcaaatacagtctcgtctctgggcgacaattccgttctgacacagcccacctcatttacacaggatgactgggagaacaggacgttcagctttggttcgctcgtgttaaaagatacagactttggtctgggccgcaatgccacactgaacacaaccatccaggcagataactccagcgtcacgctgggcgacagtcgggtatttatcgacaaaaaagatggccagggaacagcatttacccttgaagaaggcacatctgttgcaactaaagatgcagataaaagcgtcttcaacggcaccgtcaacctggataatcagtcagtgctgaatatcaatgagatattcaatggcggaatacaggcgaacaacagtaccgtgaatatctcctcagacagtgccgttctggagaactcaacgctgaccagtaccgccctgaatctgaacaagggagcaaatgttctggccagtcagagttttgtttctgacggtccggtgaatatttctgatgccaccctgagtctgaacagccgtcctgatgaggtatctcacacacttttacctgtatacgattatgccggttcatggaacctgaagggagacgatgcccgcctgaacgtggggccgtacagtatgttgtcaggtaatatcaatgttcaggataaagggactgtcaccctcggaggggaaggggaactgagtcctgacctgactcttcagaatcagatgttgtacagcctgtttaacgggtaccgcaatacctggagcgggagcctgaatgcaccggatgccaccgtcagcatgacagacacccagtggtcgatgaacggaaactccacggcaggaaatatgaaacttaaccggacaatagtcggttttaacgggggaacatcatcgttcacgacactgacaacagataatctggacgcggttcagtcagcatttgtcatgcgtacagaccttaacaaggcagacaaactggtgataaacaagtcggcaacaggtcatgacaacagcatctgggttaacttcctgaaaaaaccctctgacaaggacacgcttgatattccactggtcagcgcacctgaagcgacagctgataatctgttcagggcatcaacacgggttgtgggattcagtgatgtcacccccacccttagtgtcagaaaagaggacgggaaaaaagagtgggtcctcgatggttaccaggttgcacgtaacgacggccagggtaaggctgccgccacattcatgcacatcagctataacaacttcatcactgaagttaacaacctgaacaaacgcatgggcgatttgagggatattaacggcgaagccggtacgtgggtgcgtctgctgaacggttccggctctgctgatggcggtttcactgaccactataccctgctgcagatgggggctgaccgtaagcacgaactgggaagtatggacctgtttaccggcgtgatggccacctacactgacacagatgcgtcagcaggcctgtacagcggtaaaacaaaatcatggggtggtggtttctatgccagtggtctgttccggtccggcgcttactttgatttgattgccaaatatattcacaatgaaaacaaatatgacctgaactttgccggagctggtaaacagaacttccgcagccattcactgtatgcaggtgcagaagtcggataccgttatcatctgacagatacgacgtttgttgaacctcaggcggaactggtctggggaagactgcagggccaaacatttaactggaacgacagtggaatggatgtctcaatgcgtcgtaacagcgttaatcctctggtaggcagaaccggcgttgtttccggtaaaaccttcagtggtaaggactggagtctgacagcccgtgccggcctgcattatgagttcgatctgacggacagtgctgacgttcacctgaaggatgcagcgggagaacatcagattaatggcagaaaagacggtcgtatgctttacggtgtggggttaaatgcccggtttggcgacaatacgcgtctggggctggaagttgaacgctctgcattcggtaaatacaacacagatgatgcgataaacgctaatattcgttattcattc

SEQ ID NO: 93 - DNA, 1047 bp, Escherichia coli
atgattacactttttcgactactggcgattctttgccttttttttaacgtttcagcttttgctgttgattgctatcaggatgggtacagaggaacaaccctcataaatggagatttaccaacgttcaaaattccagagaatgcgcaacctgggcaaaaaatttgggagagcggagatattaatatcacagtttattgtgacaatgcaccaggatggtcaagtaataacccatcagaaaatgtctatgcctggatcaaattgccccaaataaatagtgccgatatgttgaataatccgtatttaacatttggcgtgacttataatggtgtagattatgaagggacaaatgaaaaaattgatactcatgcgtgcctggataaatatgaacaatactataatgggtattatcatgaccctgtatgcaatggcagcactcttcaaaaaaatgtaacatttaacgcccattttcgcgtctatgtaaaattcaaaagccgcccggcaggagatcagacggtaaactttggcacagtcaacgtgctgcaattcgacggtgaaggcggggcgaacatggcccccaacgcgaaaaatttacgctatgcgattacggggttagataatatttcattccttgactgtagtgtcgacgtccgcatttccccggaaagtcagatagtcaattttgggcagatcgctgcgaattccattgcaactttcccaccgaaggcagcattcagcgtttctaccataaaagacattgcgtctgattgtaccgaacagtttgatgttgcaaccagtttctttacttcagatacattatatgacaatacgcatctggaaataggtaacggcttgctcatgcgaattactgatcaaaaaacgcaagaagatattaaatttaaccagttcaaattatttagtacttatattcccggtcagagtgcggcaatggcaacccgcgattaccaggccgaattaacccaaaaacctggtgaaccactcgtctatggcccatttcagaaagacctgatagttaaaatcaactaccac

SEQ ID NO: 94 - DNA, 2520 bp, Escherichia coli
atgaacaataaaaacacgttttcccgggataagttatcccatgcaattaaaaatgccctgtctggcgttgtgtgttccctactcttcgttttgccagtccacgccgtagaattcaacgtcgatatgattgacgcagaagaccgtgagaatatcgacatctctcgttttgagaaaaaaggctatatcccccctggtagatacctcgttcgtgtgcaaataaataaaaatatgttgccacaaacgttaatactggaatgggtaaaagccgataatgaaagtggttcgttactctgcttaaccaaagaaaatttgactaatttcggtcttaatacggaatttattgaatcattgcaaaacatagctggcagcgaatgtctcgatttaagccaacgtcaggagttaacgacacgacttgataaagctacgatgatattatcgctaagtgttccccaggcatggttaaaataccaggcaacaaactggacgccaccagagttttgggataccggtatcaccgggtttatccttgattacaacgtgtacgccagccagtatgccccacatcacggagacagcacccaaaacgtcagctcctatggtacgttaggctttaacctcggcgcatggcgcttacgtagcgattaccaatataatcagaattttgctgatggacgctcggtaaaccgcgacagcgaatttgcgcgaacttatctgtttcgccctatcccctcctggtcgtcaaaattcactatgggccagtacgacctgagctccaatctttacgataccttccactttactggcgcatcgctggaaagtgatgaaagcatgctgccgccagatttacagggttatgcgccacaaattaccggcatcgcgcagaccaacgcgaaagtaactgtggcacaaaatggtcgtgtactttatcaaaccactgtcgcgccaggcccttttactatttctgatttggggcaatcgtttcaggggcagctggatgtcacagtggaagaagaagatggccgcaccagcaccttccaggttggctccgcatccattccctatttaacccgtaaagggcaagtgcgctataaaacgtcactgggaaaaccgacatccgtcgggcataacgatatcaataatccctttttctggacggcggaagcctcctggggctggctgaacaatgtgtcgttgtatggtggtggcatgttcaccgctgatgattatcaggctatcactaccggtattggctttaaccttaaccaattcggttcgctttcttttgatgtcactggagcagacgcgtctttacagcaacaaaatagcggcaatctgcgtggttacagctatcgcttcaactatgcaaagcatttcgaatcgacaggcagtcagattaccttcgcgggttatcgcttctcagataaagattacgtgtcgatgagtgagtacctcagctcgcgtaatggcgatgagtcaatcgataatgaaaaagagagttatgtcatttccttgaaccagtactttgaaacgctggaattaaactcttatctcaacgttacacgcaatacttattgggacagcgccagcaataccaactactccgtatctgtaagcaaaaactttgatattggcgatttcaaaggtatatctgcatcgctggcagtaagtcgaatccgctgggatgacgacgaagagaatcaatattacttctctttctctctacctttacaacaaaaccgcaacatctcctacagtatgcagcgaacgggaagcagtaatacttcgcagatgatttcctggtacgattcatcagatcgcaacaatatctggaatatttcagcgtcggcaacggacgacaatatacgtgatggcgaaccaacactgcgcggcagctaccagcactattcgccgtggggacgcctgaacattaatggcagtgtacagccgaatcagtacaattctgttaccgcaggctggtacggttcacttaccgctacacgtcatggtgtcgcccttcacgattatagctatggcgataacgcccgcatgatggtcgataccgatggcatctccggcattgaaatcaactctaaccgtaccgttaccaacgggctgggcatcgccgtgataccttcgttatcgaactacaccacctccatgttgcgggtgaacaataacgatctgccagaaggtgtcgatgtcgaaaactcggttattcgtactacgctcacccagggtgccatcggctacgcaaaactgaatgccaccaccggataccaaatcgtcggcgttattcgtcaggaaaatggccgcttccctccactaggtgtgaatgtcacggataaagcgacaggtaaagatgtgggcctggtagcggaagatggcttcgtttatctcagcggtattcaggaaaacagtattctgcatttaacctggggtgataatacctgtgaagtcacgccgccaaaccaaagtaacattagtgaaagcgcgataattttaccttgtaaaacagtcaaa

SEQ ID NO: 95 - DNA, 507 bp, Escherichia coli
ttgatgaacacaaaacagtctgttgctcaactcgccgtaccgcaccgcaagcgcctttcatcaacgatggtggtggcgctgttactttgtgtggttgctggcgcggtgatgattaatgccgctgattttccagcaactgccattgaaacggatcccggtgcaagtgccttccctaccttctatgcctgtgccctgattgtgctcgctgtcttgctggtgatacgcgatcttttgcaggcaaaaccagcctcttgcgccaacgcacaggaaaaaccggcattcaggaaaacagcaacaggaattgcggcaaccgcgttttatattgtggcgatgagctactgcggttatctcattactactcctgttttcctcatcgtcattatgacgttgatgggctacaggcgatgggtactcacaccgggtattgcgctgctgttaacggcaatcctctggttgctgtttgtcgaagcgttacaggtgccattgcctgtcggcacatttttcgaa

SEQ ID NO: 96 - DNA, 933 bp, Escherichia coli
atggtacttcttgcaggcgctgccctcagcattgcgcctgtacaggcagcctcctacccaaccaaacagatcgagttagtcgttccctacgctgccggaggcggtacggatctggttgcccgtgcctttgctgatgccgccaaaaaccatttacccgtcagcatcggggttatcaataaacctggcggaggcggtgctatcggcctgagtgaaatcgccgctgcccgccctaacggttacaaaattggtttaggcacggttgaactgaccacccttcccagcctcggaatggtgcgttttaaaaccagcgactttaaacccattgcccgtctgaatgcggatccggctgctatcacagtccgtgccgatgcgccgtggaatagctatgaagaatttatggcttactccaaagcgaatcccggaaaagtacgcattggtaactcaggcaccggagctatctggcatctggcggcagctgcactggaagacaaaacgggcacaaagttttctcatgtcccgtatgacggcgcagcccctgccattacaggcctgttaggcgggcatattgaagcggtttccgtaagcccaggagaagttatcaaccatgtgaatggcggcaagctgaagacactggtagtgatggcggatgagcgaatgaaaaccatgcctgacgtcccgacgttaaaagagaaaggcgttgatctctccatcggcacctggcgcggcctgattgtgtcgcaaaaaacgccgcaggatgtggtggatgttctggcaaaggcagcaaaagagacggctgaagagcctgcattccaggatgcactgcaaaagttgaatctcaactatgcatggcttgacgctgccagcttccagacccaaatcagcgaacaggaaaagtactttgacgagttgctgactcgcctgggcctgaaaaaa

SEQ ID NO: 97 - DNA, 2166 bp, Escherichia coli
atgctgcgatggaaacgctgtattattctaacatttatctctggtgctgctttcgcggcgccagagataaatgttaagcaaaacgaatcgttacctgatttaggtagccaggcagcacaacaggatgaacaaaccaacaagggtaaatcgctgaaagagcgcggagccgattacgtcatcaactccgccacgcaagggtttgaaaacttgacccctgaggcgctggaatctcaggccagaagctatctgcaaagtcaaatcacctcaaccgcacaatcttatattgaagacacactctctccctacggtaaggtccgtttgaacctctccattggtcagggcggcgatctggatggcagttccatcgattattttgttccctggtacgataatcaaaccactgtttatttcagccaattttctgcgcaacgaaaagaagatcgtacgatcgggaatattggccttggggtaaggtataattttgataaatatctattgggtggaaatatattttatgattatgactttacccgtggacatcgccgtttaggtttaggcgccgaagcctggacggattatttaaaattctcaggcaactattatcacccactttctgactggaaagactctgaagatttcgacttttatgaagaacgccctgcgcgcggttgggatattcgtgccgaagtctggttaccttcttatccgcaactggggggcaaaattgtcttcgagcaatattacggcgatgaagtcgccctttttggtacggataatttggagaaagatccctacgcggtaacgcttggactgaattatcaaccagtgccgttactgacagttgggacggactataaagcggggaccggagataacagtgatgtcagcattaatgccactcttaattatcagttcggcgttccgctaaaagatcaattggatagcgataaagtgaaagcggcgcactcgctgatgggcagccgtcttgatttcgttgagcgtaataactttattgttctggaatacaaagaaaaagatccgcttgatgtcaccctgtggttgaaagcggatgccaccaacgagcaccctgagtgcgtcattaaggacactcccgaagcggccgtcggtctggaaaaatgtaagtggaccattaacgcactcattaatcatcattacaaaatcgttgcggcctcctggcaggcgaaaaacaatgccgcccgcacgctggtgatgccggttatcaaagagaatactctgacagagggtaacaataaccactggaacctggtgctgcctgcctggcagtacagttccgatcaagccgaacaagaaaaactcaatacctggcgagtacgtctggcgctggaagatgaaaagggcaaccgacagaactctggcgtggtggaaatcaccgttcagcaggaccgtaaaatagagttgattgttaataacatcgcgaacccagaagagaacaaccacagccacgaagccagcgcacaggcagatggcgttgatggtgtagtgatggatctcgatgtaaccgacagctttggcgataacaccgaccgcaacggcgatgcgttgccggaagataaccttacgcctcagctttacgacgcgcaggacaaacgagtgacgttaaccaacaagccctgctcgaccgataacccctgcgtttttattgccaaacaagataaagaaaagggcactgtcaccctctccagtaccttacctggcacctatcgctggaaagcaaaagccgcgccctacgatgacagtaactatgtggatgtcactttcctcggggcagaaattggtgggctaaatgcttttatctatcgtgtgggggcggctaaacccagcaacctgataggtaaagataaagaaccgttgccgtcaacaacatttatcgatttgttttatggcgcgacaacaataaagacggtgtcttccagcaggtcgaaaaacctgacgaagagatggtgcagtacgactacaagtgggaatttaccggcaagagcatcaatggtaagtgggtgcacaggcgaacactccaatgaggacattgtgattccggccactaaccgtgaagcggcgcaaacctatggcgcacaagcgggagatggcttgcagggatacggtttacgcgtgctgtataccaaaaaa

SEQ ID NO: 98 - DNA, 957 bp, Escherichia coli
atgaagcaggataaaagacgcggtctgacccggatcgcattagcgctggcactggcaggttattgtgtggcacctgtggcgctggctgaagacagcgcctgggtcgacagcggtgaaaccaatattttccaggggaccattccgtggctctattcggaagggggaagtgctacgacagatgccgaccgtgtaacgttgacttctgatctaaaaggcgctcgcccgcaaggcatgaaacggacaagcgtttttactcgggtgataaatattggtgataccgaaggcgacgtggatcttggtggattgggcgataacgcgaaaactatcgatactatccgctggatgagctacaaggatgcgcaggggggggatccaaaagagctggcaacgaaggtgaccagttacactcttaccgatgccgaccgtggtcgctatatcggtattgaaattacgccaaccacgcagaccggtacgccaaacgtcgggactgcgctgcatctttatgacgtttctactgccagcggcggcggaagcgacagcgataacgttgcaccggggccggtggttaaccagaacctgaaagtcgccatctttgttgatggtaccagtatcaaccttatcaacggtagcacaccaatcgaacttggcaaaacctacgtggccaaactgtactcggatgagaacaaaaatggcaagtttgatgcgggtaccgatgctgacgtcaccgccaattatgacttccgttgggtactttctggcagcagccaacagcttggcacttcgggtggcatcgttaactcaagcttcgataataacaatttggtcatccctgcgaccaacgacgaagccagaaccaaccttaacggccctgcgcgcgatggaaaagaggcactttccatcccgaccaacggcgacggggtacagggttacaaacttcacattatttacaaacacaaa

SEQ ID NO: 99 - DNA, 1887 bp, Escherichia coli
atgaagaaagtgctcactctctcactactggctctgtgtgtgtctcatagtgcagtagcagcaaactatacgttcaataacgataatattgccctctcgtttgatgatacaaactcgacgattgtgctgaaggaccgtagaactaaccatccgatcacaccacaggaattgttctttctgacactaccggatgagacaaaaatccacaccgcagatttcaaaatcaagcacatcaaaaaacaggacaatgcgattgtcatcgactttacgcgcccagattttaacgtaacagtgcagttgaaccttgtgaagggaaaatatgccagcatcgactacactattgccgccgttgggcaaccacgagacgtcgccaagattaccttcttcccgaccaaaaaacagtttcaggctccttacgtagacggcgcaatcactagctcaccgatcattgcggactcgttctttatcctgccgaataaaccgatcgtgaatacctacgcctatgaagcaacaaccaatctcaacgtagaactgaaaactccaattcagccagagacgccggttagctttaccacctggttcggtactttcccggaaaccagccagttgcgacgcagtgtgaaccagtttattaatgccgtacgtccacgtccgtacaagccttatttgcattacaacagttggatggatatcggctttttcactccgtacaccgaacaggatgttctgggacgcatggacgaatggaacaaggaattcattagcggccgcggagtggcgttagacgcttttctgctggacgatggctgggacgatcttaccggacgctggttatttggcccggcattcagcaacggttttagcaaagtacgagagaaagccgatagcctgcacagctccgttgggctatggctttcaccgtgggggggttacaataagccgcagcgacgttcgcgtttcgcatgcaaaagagtatgggttcgaaaccgtggacggcaagctggcgctttcgggagcgaactacttaaaaacttcaatgagcagatcattaatcttatcaaaaatgaacacattacctcgtttaaactcgacggaatggggaacgccagttcacatataaagggtagcccgttcgcctcggattttgatgcgtcaatagctctgctgcacaatatgcgcagagcaaacccgaatctatttatcaacctgaccaccggcaccaacgccagcccgtcctggttgttctatgctgattctatctggcgtcagggggatgatataaacctgtatggccccggcacgccggtgcagcagtggataacatatcgtgatgccgagacataccgctctattgtacgtaaaggcccgctattcccgctgaactcgctgatgtaccacgggatagtcagcgccgagaatgcctattacgggttagagaaggtgcaaacggacagcgactttgccgatcaggtctggagctacttcgcgaccggcacccagctgcaggagctgtatattaccccgtccatgctgaacaaggtgaagtgggatacgctggcgaaggctgcaaaatggtcgaaggaaaatgccagcgtgctggttgatacccactggattggcggcgacccaacggcgcttgccgtgtacggctgggcatcctggagcaaagacaaagccattctcggtttgcgcaacccatcggataagccacagacctactatctggatttggcgaaggatttcgaaataccggcaggaaacgcggcgcagtttagtctgaaagcggtatacggcagcaataaaacagtgcccgttgagtataaaaacgcgacggtgattacgttgcagccgctggaaacgctggtgtttgaggcggtgaccattaac

SEQ ID NO: 100 - DNA, 5334 bp, Escherichia coli
atgaacaaaatatttaaagttatctggaatccggcaacaggcagttacaccgttgccagcgaaacggcgaagagccgtggtaaaaaaagcgggcgcagtaagctgttaatttctgcactggttgcgggtgggttgttgtcgtcgtttggggcaagtgcagataattacactgggcagccaactgattatggcgatggctcagcaggtgacggctgggttgctatcggtaaaggggcaaaagcaaatacctttatgaacactagtggcgcgagtacagctttaggatatgacgcgatagccgaaggtgagtacagttctgccatcgggtcaaaaacccttgcaactggtggagcatccatggcgttcggggttagtgcaaaagcaatgggtgacagaagtgtcgcgctaggtgcatcgtcagtagcaaatggcgatcgttcgatggcttttggtcgttacgcaaagacgaatggttttacatctcttgctattggggactcctcccttgccgatggtgaaaaaactattgcgttaggaaatacggctaaagcttacgaaattatgagcatcgccctcggtgataatgccaatgcgtcaaaagagtatgcaatggcgctgggagcaagtagcaaagctggcggtgctgatagcctcgcattcggcagaaaatctacagctaatagcactggctcactggcaataggtgctgacagtagcagttcgaacgataacgccatcgcgatagggaacaaaacgcaagccctgggagtgaattcgatggccctgggtaatgcaagtcaggcatctggcgaatccagtattgcattaggtaacaccagtgaagccagcgaacaaaatgcgattgcgctggggcaaggtagcattgcaagcaaagtgaactcaatcgcgttgggaagtaacagtttgtcctcgggagagaatgccatcgcattgggagagggtagtgccgctggtggcagcaacagccttgctttcggtagccagtccagggcaaacggcaatgattctgtcgccatcggtgtaggggctgcagcagcgaccgacaattctgtcgctatcggcgcaggatcgaccacagatgcaagcaatacggtttcagttggcaacagcgcaacaaaacgcaaaattgttaatatggctgctggtgccataagcaacaccagtaccgatgccatcaacggctcacagctttatacgatcagtgattcagtcgccaagcgactcggaggaggcgctactgtaggcagcgatggcaccgtaaccgcagtaagctacgcgttgagaagcggaacctataataacgtgggtgatgctctgtcaggaatcgacaataataccctacaatggaataaaaccgcgggggcgttcagcgccaatcacggtgcaaatgccaccaacaaaatcactaatgttgctaaaggtacggtttctgcaaccagcaccgatgtagtaaacggctctcaattgtacgacctgcagcaggatgctctgttgtggaacggcacagcattcagtgccgcacacggcaccgaagccaccagcaaaatcactaacgtcaccgctggcaacctgactgccggcagcactgacgccgttaacggctctcagctcaaaaccaccaacgacaacgtgacgaccaacaccaccaacatcgccactaacaccaccaatatcaccaacctgactgacgctgttaacggtctcggtgacgactccctgctgtggaacaaagcagctggcgcattcagcgccgcgcacggcaccgaagccaccagcaaaatcaccaacgtcaccgctggcaacctgactgccggtagcactgacgccgttaacggctcccagctcaaaaccaccaacgacaacgtgacgaccaacaccaccaacatcgccactaacaccaccaatatcaccaacctgactgacgctgttaacggtctcggtgacgactccctgctgtggaacaaaacagctggcgcattcagcgccgcgcacggcactgacgccaccagcaagatcaccaacgtcaccgctggcaacctgactgccggcagcactgacgccgttaacggctcccagctcaaaaccaccaacgacaacgtgacgaccaacaccaccaacatcgccactaacaccaccaatatcaccaacctgactgacgctgttaacggtctcggtgacgactccctgctgtggaacaaaacagctggcgcattcagcgccgcgcacggcactgacgccaccagcaagatcaccaatgtcaaagccggtgacctgacagctggcagcactgacgccgttaacggctctcagctcaaaaccaccaacgataacgtgtcgaccaacaccaccaacatcaccaacctgactgacgctgttaacggtctcggtgacgactccctgctgtggaacaaaacagctggcgcattcagcgccgctcacggcactgacgccaccagcaagatcaccaatgtcaaagccggtgacctgacagctggcagcactgacgccgttaacggctcccagctcaaaaccaccaacgataacgtgtcgaccaacaccaccaacatcactaacctgacggattccgttggcgaccttaaggacgattctctgctgtggaacaaagcggctggcgcattcagcgccgcgcacggtaccgaagctaccagcaagatcaccaacttactggctggcaagatatcttctaacagcactgatgccattaatggctcacaactttatggcgtagcggattcatttacgtcatatcttggtggtggtgctgatatcagcgatacgggtgtattaagtgggccaacctacactattggtggtactgactacactaacgtcggtgatgctctggcagccattaacacatcatttagcacatcactcggcgacgccctactttgggatgcaaccgcaggcaaattcagcgccaaacacggcattaataatgctcccagtgtaatcactgatgttgcaaacggtgcagtctcgtccaccagcagcgacgccattaacggttcacaactttatggtgttagtgactacattgccgatgctctgggcgggaatgctgtggtgaacactgacggcagtatcactacaccaacttatgccatcgctggcggcagttacaacaacgtcggtgacgcgctggaagcgatcgataccacgctggatgatgctctgctgtgggatacaacagccaatggcggtaacggtgcatttagcgccgctcacgggaaagataaaactgccagtgtaatcactaacgtcgctaacggtgcagtctctgccaccagcaacgatgccattaatggctcacagctctatagcactaataagtacatcgctgatgcgctgggtggtgatgcagaagtcaacgctgacggtactatcactgcaccgacttacaccattgcaaataccgattacaacaacgtcggtgaagccctggatgcgctcgataataacgcgctgctgtgggatgaagacgcaggtgcctacaacgccagccatgatggcaatgccagcaaaatcaccaacgttgcggctggtgatctctccacaaccagtaccgatgctgttaacggttcccagttaaacgcaaccaatattctggttacgcaaaatagccaaatgattaaccagcttgctggtaacactagcgaaacctacatcgaggaaaacggtgcgggtattaactatgtacgtaccaacgacagcggcttagcgttcaacgatgccagcgcttcaggtattggcgctacagctgtaggttataacgcagttgcctctcatgccagcagtgtagccatcggtcaggacagcatcagcgaagttgatacgggtatcgctctgggtagcagttccgtttccagccgtgtaatagttaaagggactcgtaacaccagcgtatcggaagaaggtgttgtgattggttatgacaccacggatggcgaactgcttggcgcgttgtcgattggtgatgacggtaaatatcgtcaaatcatcaacgtcgcggatggttctgaagcccatgatgcggtcactgttcgccagttgcaaaacgccattggtgcagtcgcaaccacaccaaccaaatactatcacgccaactcaacggctgaagactcactggcagtcggtgaagactcgctggcaatgggcgcgaaaaccatcgttaatggtaatgcgggtattggtatcggcctgaacacgctggttctggctgatgcgatcaacggtattgctatcggttctaacgcacgcgcaaatcatgccgacagcattgcaatgggtaatggttctcagactacccgtggtgcgcagaccaactacactgcctacaacatggatgcaccgcagaactctgtgggtgagttctctgtcggcagtgaagacggtcaacgtcagatcaccaacgtcgcagcaggttcggcggataccgatgcggttaacgtgggtcagttgaaagtaacggacgcgcaggtttcccagaatacccagagcattactaacctgaacactcaggtcactaatctggatactcgcgtgaccaatatcgaaaacggcattggcgatatcgtaaccaccggtagcactaagtacttcaagaccaacaccgatggcgcagatgccaacgcgcagggtaaagacagtgttgcgattggttctggttccattgctgccgctgacaacagcgtcgcactgggcacgggttccgtagcagacgaagaaaacaccatctctgtgggttcttctaccaaccagcgtcgtatcaccaacgttgctgccggtgttaatgccaccgatgcggttaacgtttcgcaactgaagtcttctgaagcaggcggcgttcgctacgacaccaaagctgatggctctatcgactacagcaacatcactctcggtggcggcaatagcggtacgactcgcatcagcaacgtttctgctggcgtgaacaacaacgacgcagtgaactatgcgcagttgaagcaaagtgtgcaggaaacgaagcaatacaccgatcagcgcatggttgagatggataacaaactgtccaaaactgaaagcaagctgagtggtggtatcgcttctgcaatggcaatgaccggtctgccgcaggcttacacgccgggtgccagcatggcctctattggtggcggtacttacaacggtgaatcggctgttgctttaggtgtgtcgatggtgagcgccaatggtcgttgggtctacaaattacaaggtagtaccaatagccagggtgaatactccgccgcactcggtgccggtattcagtgg

SEQ ID NO: 101 - DNA, 681 bp, Escherichia coli
atgaacctaaagaaaacactgttaagcgtgttaatgatattgcaactttgcttattggtagggtgtgactatattgaaaaagcgagtaaggtcgacgatctcgttacacagcaagagttgcaaaaaagcaaaattgaggcgcttgaaaaacaacaagaactcgacaagcgcaagatagaacactttgaaaaacaacaaactaccatcataaacagtaccaaaacgctcgctggtgtggtgaaggcagttaaaaacaaacaggacgaatttgtctttacagaatttaacccggcacaaacccaatactttattttaaataacggctctgttggtttggcagggaaaatactgtctattgacgcagtagaaaacggcagtgttattcgtatttcactggttaacttattaagtgttcctgtatcaaatatgggtttctacgcaacatgggggggagaaaaacccaccgacatcaacgcattagcaaaatggcagcaattgctatttagtaccgcaatgaactcctccctgaaattattaccaggtcaatggcaagacattaatttgacgctaaaaggtgtctcgcccaacaacctcaaatatctgaaattagccatcaacatggcaaatattcagttcgaccgtcttcaacctgctgaatctccacagcggaaaaacaaaaaa

SEQ ID NO: 102 - DNA, 3327 bp, Escherichia coli
atgaaaagagttgtgcgtcttttgggtgtggggttactgctccttgttgtgttgttgctcattttgtttgttctggctcagaccacaccgctgatatcagcacaggatgagcatgctgtctggcttcgtctgttgataacagcgattgtgatctgtttgctaagtatgtgcatatttttcctcttttctttccggcagaacgaagcctcgacgatatcactatacgctcaaccgactgatataaaggaaataaatacggagcagccgaactatgcatcactgctgacgatatatttacgcgaccgctacggtccgttctggcggcgtaaagtccgcctgctgctggtgaccggcgagcctgaacaggcagaagccatcgcgccggggctgaccgggcaacactggctggaaggcgaccacacggtgctgatatatggcggcaggccaacagcggagcctgatgtcacactgctgaccgccttaaaaaaactgcgccgcagccgtccgctggacggcatcatctgggcgctgacagaagaacagagccgccagacagcgcaactcgacaaaggctggcgcggactgataaacggcggtaagcgactcggttttcaggctccactctatttgtggcaggtctgtgacgacggtgattatcagaccggacgccccctgcaaagcgtcggctgcctgctgccggaacgctgtaccccggaacaactggctgtaatgctggaagcagccgctgacggaacagggcatgtcgcagctactgaccgataccgcatgttttctgctgcgtctggctcatacccttgcagagcggggtattgctcactggcagaccgtcctgaaaccgctgctggcaggcggcgcattttcttccctgcgcctgcgcggcctgatgttcagcccgccgcttgccgccgtgccggaggccagcacctcatgcagtggctgccgtcaccggtctgggcgggcgtgacggtgataacgcgcgcgggcgcacggtgggttttcctgtggctgcgtaccgcactgatgtccgctgtctgcgtgctggtgatatggggggccggaatgacgacctcgttcttcgccaaccgcgctcttgttcaggaaaccggtatccagacggcacgtgcgcttgatacccgcctgccgctggcagaacaactggtggcgctgcataccctgcagggcgaactggaacgcctgcaatatcgtatccgcgaaggtgcgccgtggtatcagcgttttggccttgaacgtaaccaacaactgctcgccgccgcttttcccggctatgcgcaggcggcaaaccggctggtgcgcgacgtggccgttgaccatctgcaacagcaactgaacgcctttgtcgccctgccgcccaacagtcctcagcgtaccgccaccggtgaacaacgctataagcagcttaaggcattgctgatgacttcccgcccggaaaaggccgacgctgcctttttcagtaccacgctgatggcggacggtctgcgctacgagaatatcccggaaggtgtgcggcagagcgtgttgccgtcactgctgaccttctggacggcgaacctgccggaacacccgcagtggaaaacatcgccgccaccggaactgaccggcgcagtgcgtaaaatcctgctgcgccagattggtgtgcgtaatgccgaaaacaccctctaccagaacgtgctgcaacaggtgtcccgcaactacgccgatatgacgctggcggacatgaccggggataccctcaccgaatctcttttcagtacggaacagacggtgccggggatgttcacccgtcaggcgtgggaaggacaggtcagggaagccatcgagcaggtggtgacggcgcggcgcgaggaaatcgactgggtactcagcgaccgccagcaggatacctctgcggatatctcgccggatacgctgcgtaaccgtctcacctcacgctactttaccgactttgccggaagctggctggcgtttctcaacagcattcactggaaaaaggaagactcgctctccggcattctcgaccagctgacactgatggccgatgcccgtcagtcgccactgattgcgctgacggacaccctcgcgtggcaggcggcgacaggcagggaaaaccgtggtctgtcagactcgctggcgaaatcggcacaggaactgtttaacggcaaggagaaaacgccgcagcaatcccgtgaaggtgacgacgtgcctgtcgggccgctggataaaaccttcacgccgctgctgcgtttgctgggcgataaggccggaggcggcgacagccagctgagtctacagacctacctcacccgcgtcacccgcgtgcgcctcaaactgcaacaggtgaccaacgcccccgacccgcaggagatgacccaacaactggcgcagacggtcttacagggtaaaaccgttgacctcaccgacacccgcgactacggacggttaatcgccgccagtctgggcgaagaatggagtggcttcggtcaggcgctgttcgttcgcccggtagagcagtcgtggcggcaggtgctgacgcctgcggcggacagcctgaaccgccagtggcagcgggcgattgtcagccactggaatcaggacttcgctggccgctatccgttcaaagcctcacagaacgatgcctccctccccctgctggcgcagtacctgcgcgatgacgggcgcatcaacctgtttatcgccgccaacctttccggcgtgctgaaacgagagggccgctactgggtggctgacgccatgaacacgcaggggctgacggtcaatccggactttatccgcgccctgaaccgcctgcgcgacgtggccgataccgcctttgccagcggcgatgccgggatacattttgaactgcgggcaaaaccggcgcgtgacgtgatgaagacgcatctggtgattgacgggcaggagctggaatatttcaaccagaaagaacgctggcagcgttttaactggccggatgaacagtggcaacccggcgcatcgctaagctggaccagcacacaggcgatggagcgcatactggcggattaccggggaagctggagtcttattcgcctgctggaacaggcgcaggtgacgccggtggacagcagcacctttaaggtggtgtggaaagcgcaggacggcctgccgctgaattacctgctacgggttgaacagggtaaagggccgctggcgctgctggagctgaaaaacttccgcctgccgggacaggtgtttctgaccggaaaaagtatgaaggatgtggaagagtatggggaagacgccgatgag

SEQ ID NO: 103 - DNA, 534 bp, Escherichia coli
atgtttcctattcgttttaaacgtccggcgttgctctgtatggcgatgctgacggttgttctgagtggctgcggcctgattcagaaagtggtggatgaatcgaaaagcgtggcctcagccgttttctacaaacaaatcaaaatactgcatctcgatttcttctcccgcagcgccctgaatacggatgcggaagatacgccgctttccacgatggtgcatgtctggcaactgaaaacccgcgaagattttgacaaggcggattacgacaccctgtttatgcaggaagagaagacgctggagaaggacgtactggcaaaacacaccgtctgggtaaaaccggaaggcacggcatccctgaatgtgccgctggataaagagacgcagtttgtcgccattattgggcagttttatcaccctgatgaaaaaagcgacagctggcgtctggtgatcaaaagggacgaactggaggccgacaagccgcgctcgattgaactgatgagaagcgacctgcgactgctgcctctcaaggataaa

SEQ ID NO: 104 - DNA, 840 bp, Escherichia coli
atgatttcagggggaaatatgttgaaagaatggatgatatttacgtgcagtttattgactctggctggggcgtcactgcccctcagtggctgtatttccagaggccaggagtctatatccgaaggggcggcatttggggcagggatcctgcgcgaaccgggagcaacaaaaaaagccgacacgaaagacctcaatgtgccaccaccggtttatggtccgccgcaggtgatatttcgcattgatgacaaccgctatttcacgctagaaaattatacccactgcgagaacgggcagacgttttataataataaagcaaaaaacattcatgttaaaatattagacgcttcagggtatttatttaaaggccgcttattctggttatcaacgcgtgatgattttctggcctttcctgccacgttaaataccagacacgcttcctgtatggggtcgaataaaggctgtatgaatgcggtcattgtcactaccgatggtggaaaaagacgcagtggtgtgccatacggcagttatacccagaatccgaccggtgccacgagggattatgacatgctggtgatgaatgacggcttctacctgcttagatatcgggggggacagggcagatttagtccggtgatacttagatggattctcagtactgaagatagctctggtgttgtgcgttcagaagatgcttatgaattgttccgtcccggagaagaggtaccctccaccggtttttataaaatcgacctgtcacgtttttatcccaaaaacaacgttatggaaatgcagtgtgacaggacgctggagccagttcaaccttcagagagtaaaattcaa

SEQ ID NO: 105 - DNA, 1503 bp, Escherichia coli
atggaacacgttagcattaaaacattatatcatctcctgtgctgtatgctgctctttatttccgctatgtgcgctttggcgcaagaacatgagcctatcggggcgcaagatgagcgcctgtcgacattaattcaccaacggatgcaggaggccaaggtcccagccctttccgtaagtgtgaccattaagggggtacgtcagcgatttgtctacggtgttgccgatgtggctagtcagaaagcgaatactctagacacagtttacgagctgggatcgatgagtaaggcgtttaccggacttgtggtgcaaatactgattcaggaaggcagactccggcaaggggatgatatcattacctatctgccggaaatgcgcttgaattatcagggaaaacctgcttccctgaccgtggctgatttcctttatcatacatcaggattgcctttttcaacactggctcggctggaaaaccctatgcctgggagcgctgtggcacagcaactgcgcaacgagaatctgctgtttgcgccgggtgcgaagtttagctatgcctccgccaattatgatgtgttgggcgcggtgattgaaaatgtgacgggaaaaacctttacagaggtcattgcggaacgactcacgcagccgctgggcatgtcggcgactgtggcagttaagggggatgagattattgtcaacaaggcaagcggctataaactgggattcggcaaacccgttctgtttcatgcgcctctggcccggaaccatgttcctgccgcctatatccatagcactctgcctgatatggaaatatggatagacgcctggttgcacagaaaggctttgccggcaacgctgcgtgaggcgatgagtaacagttggcgtggtaatagtgatgttccgcttgccgcagacaatcgtatcctctatgccagcggttggtttatcgaccagaatcaaggcccttacatcagtcacggtgggcagaatccaaacttttcttcttgcattgcgttgcgaccggatcagcagattggcattgttgcgctggcaaatatgaattcgaatctgatactacagctttgcgcggatatcgataattatctgcgcattggcaaatatgctgacggcgctggtgatgcaattacagccaccgatacccttttcgtctacctcacgttgttgctgtgtttttggggggcggtggttgtagtgcgcggtgctttccgtgtttatcgcgcaacggcgcatggccctggaaaacagcagaggttacgtttacgcgtacgtgactatatcatcgccttggcggttcctgggctcgtggccgccatgctctatgtcgcaccgggtatactatctccaggacttgactggcgttttatcttggtatggggtccatcgagcgtgttggcgataccgttcggaattatcctgttagctttcgttctgacattaaatcatcaaattaaacgaattctattacacaacaaggagtgggacgatgag

SEQ ID NO: 106 - DNA, 2046 bp, Escherichia coli
atgaagaacaaatatatcattgctccgggcattgccgtgatgtgttctgcagttatatcatcaggttatgccagttctgataaaaaagaagatacgcttgttgttactgcctccgggttcactcagcagctcagaaatgccccggccagtgtctcagtcattacttcagaacaactgcaaaaaaaaccggtttcagatctggtcgatgcagtaaaagatgttgaagggattagtatcactggtgggaatgaaaaaccggatatcagtatacgtggtctaagtggcgattacacgctgattctggtcgatggacgacgtcagagcggtcgggaatccagaccaaacggcagcggcggttttgaagccggatttatccctcctgtggaagcaattgaacgcattgaagtgatccgtggccctatgtcttccctgtatggttctgatgccatcggaggggtcattaatatcataaccaaaccagttaataaccaaacatgggatggcgtacttggacttggggggattattcaggaacatgggaaatttggtaactcaaccacaaatgacttctatctgtcaggcccattgattaaggataaacttggtcttcagctatatggaggaatgaactatcgcaaggaagatagtatctctcagggaacaccggcaaaagataataagaatataacggcaacgctccagtttactccgactgaaagccagaagtttgtttttgaatatggaaaaaataaccaggtgcatacattaacacctggtgagtctctcgatgcctggactatgcggggaaatcttaaacaaccaaacagtaaaagagaaacgcataattcacgtagtcactgggtagcagcatggaatgcccagggcgaaatactgcatcctgaaattgctgtttatcaggagaaagttattcgtgaggttaaatcaggtaaaaaagataaatataatcattgggatcttaattacgagtcaagaaaaccggaaataaccaacacaatcatagatgcaaaagtgacggcatttctgccggaaaatgtactgaccatcggaggtcaatttcagcatgcagagctccgtgatgactcagccacgggtaaaaaaacgacagaaacacagtctgtttcaattaaacagaaagctgtttttatagaaaatgaatatgcagcaacggattctctcgccctgactggaggactgcgtctcgataatcatgaaatctatggcagttactggaatccaagattgtacgctgtttataacctgaccgataatctcacactcaaaggggggatcgcaaaagcatttcgggctccttcaattcgtgaggtgagtcctggatttggaacactgacgcagggtggtgcctctattatgtatggaaacagggacctgaaaccggagaccagtgtaaccgaagagatcggtattatttatagtaatgatagtggtttttcggcgagcgcgacgctgtttaatactgattttaaaaataagttgaccagttacgatataggtacaaaagatccagtcaccgggttaaacacttttatttatgataatgtaggtgaggcaaatatcagaggggtggagcttgcaactcagattcctgtgtatgataaatggcatgtatctgcaaactatacatttactgactctcgtcgaaaaagtgatgacgaaagtctcaatggcaagtcgctgaaaggggaacctctggaaagaactcccagacatgcagccaatgcaaaactggaatgggattacactcaggatattacattttattcatctctgaattatacgggaaaacaaatctgggcagcacaaagaaatggtgctaaggttccccgcgttcgtaatggattcacatctatggatattggtctaaattaccagattctgccagacacgctgattaattttgccgttcttaacgtcacagacagaaagagcgaggatatcgataccattgatggtaactggcaggtcgatgaaggacgccgttattgggctaatgtaagagtatccttc

SEQ ID NO: 107 - DNA, 492 bp, Escherichia coli
atggggtttagaaaaacaataatcacttcggtaggtttgatatttatttcattctcttttgtggcaaagtgctctcaactcaaaaatttgaataattactcagtgatgctttgtggaaaagtgtcaaataatatcctggatgatattggtggttataaagaaagaaatatattaatgctgcgagctataaaaaaaatcataataatgacaatcgtaaatattatatttttctattcctttcaatcgactgcggatgaaatggttttaataaaaaaatacgggtttgggcttgagagagatatcaaaggaaggccattaatttatcctatcgaaaattatgatgagtgtaagaaaaaatgcaatcatatgaattatatagcggatgtcaatgctcaattagctatgagtaaaaaaaataacaggatttttgctaacataacctttactaacaatagctctaccacgtatttttttctaaatattatctaccta

SEQ ID NO: 108 - DNA, 654 bp, Escherichia coli
atgaatcaaattaaagataataaggtaattatgaaaataaaaaatttaatatcagtcattttactatcaggaggtattatggggactggattgtactcgagcgataaccatcaaaaaatccgcagcaggtttaatatacaggaatcatattgtgccattaagactaatggtgtccttggattcagcaaccgaaaggatgtattgcgagaaaatggtgattcaaccggaaccaccagttccagcactaatgccatgatgctgatggaaaatggtgaaaatgaaatcagtctggagattggggcgttaaggtggttttctgataaacctgccagtaccgaagaacgagggcatttctcccaaaaagcagggtgcagtctggatttggttcgttttgttaagcaggaagaaaccatactttcttcgataaaggtgaccatcaaccagcagggaatacctgaagcgcagccagacagcatgcatcctgttatccgaaaagagattctggctgagcaggcagaacccggatttattgatccagactattttaatgaaacttatttcccgaaagggatgaaggtgtatcaatttacacaaaaggtctcggtggcggggcttcctgatgggcctggacgcagtacgccctttaccggagca

SEQ ID NO: 109 - DNA, 8198 bp, Escherichia coli
atgcatcagcctcccgttcgcttcacttaccgcctgctgagttacctggtcagtgcgattatcgccgggcagccgttgttaccggctgtgggggccgtcatcaccccacaaaacggggccggaatggataaagcggcaaatggtgtgccggtcgtgaacattgccacgccgaacggggccgggatttcgcataaccggtttacggattacaacgtcgggaaggaagggctgattctcaataatgccaccggtaagcttaatccgacgcagcttggtggactgatacagaataacccgaacctgaaagcgggcggggaagcgaagggtatcatcaacgaagtgaccggcggtaagcgttcactgctgcagggctatacggaagtggccggcaaagcggcgaatgtgatggttgccaacccgtatggtatcacctgtgacggctgtggctttatcaacacgccgcacgcgacgctcaccacgggcaaacctgtgatgaatgccgacggcagcctgcaggcgctggaggtgactgaaggcagtatcaccatcaatggcgcgggcctggacggcacccggagcgatgccgtatccattattgcccgtgcaacggaagtgaatgccgcgcttcatgcgaaggatttaactgtcactgcaggcgctaaccgtgtaactgcagatggtcgtgtcagagccctgaagggcgaaggtgatgtgccgaaagttgccgttgataccggcgctctcggtggaatgtacgccaggcgtattcatctgacctccactgaaagtggtgtcggggttaatcttggtaacctttatgcccgcgatggcgatatcaccctggatgccagcggcagactgactgtcaacaacagtctcgccacgggggccgtcactgcaaaaggtcagggcgtcaccttaaccggcgaccataaagcgggaggtaacctgagcgtcagcagccggagagatatcgttctcagcaatggaacgcttaacagcgacaaggacctcagcctgaccgccggcggcagaatcactcaacagaatgaaaaactgactgccggccgggatgtaacgcttgccgcgaaaaacatcacacaggataccgccagccagattaacgcggcccgcgatatcgtgactgtcgccagtgacacgctgacaacacagggacagataaccgccgggcagaatctcacggccagcgccaccacgctgacgcaggacggaatattgctggcgaaaagtcatgcgggactcaatgccggtacgctgaataacagtggcgccgttcagggagctaccctgacgctcggcagtacaacgctcagcaacagtggctccctgctcagtggcggtcccctgaccatgaatacccgcgactttacccagagcggccgcactggcgcgaagggcaaagtggatatcatggccagtgggaaactgaccagtacaggtttgctggtgacgatgcacttggtgctgaaggcgcaggatgtgacacagaacggtgtgctgtccggcggcaaagggctgacggtcagtgcgacgagctccggtaaaaaatcggtcacccacagcgatgctgcgatgacgctgaatgtgacaacagtggcgctggacggggaaaccagtgccggtgacaccctccgggttcaggcagacaaactgagtaccgcagcgggcgcacaacttcagagcggcaaaaatctcagcatcaacgccagagatgcacgtcttgcaggtacgcaggcagcacaacagaccatggtggtgaacgccagtgaaaagctcacccacagcgggaaaagcagtgccccgtcgctcagcctcagtgcgccggaactgaccagcagcggcgtacttgttggttccgccctgaatacacagtcacagaccctgaccaacagcggtctgttgcagggggaggcctcactcaccgttaacacacagaggcttgataatcagcagaacggcacgctgtacagtgctgcagacctgacgctggatataccggacatccgcaacagcgggcttatcaccggtgataatggtttaatgttaaatgctgtctccctcagcaatccgggaaaaatcatcgctgacacgctgagcgtcagggcgaccacgctggatggtgacggcctgttgcagggcgccggtgcactggcgcttgctggcgacaccctctcacagggtagtcacggacgctggctgacggcggacgacctctccctccggggcaaaacactgaataccgcagggaccacgcagggacagaatatcaccgtgcaggcggacagatgggcgaacagtggttccgtgctggcaaccggtaaccttactgcttcggcaaccggtcagttgaccagtaccggcgatatcatgagccagggtgacaccacgctgaaagcagccaccacggacaaccggggcagtctgctttcggccggcacgctctcccttgatggaaattcactggataaccgcggcactgtccagggtaaccatgtcacgattcgccagaacagtgtcaccaacagtggcacgctcaccgggatcgccgcactgacgcttgccgcccgtatggcatcccctcaacctgcgctgatgaataacggaggttcattgctgaccagcggcgatctgacaatcaccgcaggcagtattaccagttccggacactggcagggcaaacgggtgctgatcaccgcagacagtctggcaaacagcggggcgatccaggcggctgacagcctgactgcacgtctgacgggtgagctcgtcagcacagcgggcagcaaagtcacctcgaacggtgaaatggcgctcagtgcactgaatttaagcaacagcggacaatggattgcaaaaaatctgaccctgaaggcgaactcactgaccagtgcgggtgacatcaccggtgtggatactctcacgctcacggtgaatcagacgctgaacaatcaggcgaacggaaaactgctcagtgcaggtgtgctgacgctgaaggcagacagtgtcacaaacgacgggcaattacagggaaatgtcaccaccatcacggcaggacaactcacaaacggcgggcatctgcagggcgaaacgctgacgctgacagcctccggtggcgtgaacaaccgttccggtggtgttctgatgagccggaatgcactgaatgtcagtactgcgaccctgagtaaccagagcacgatacagggtggaggcggggtttccctgaacgccacagaccgtctgcagaacgacggcaaaatcctctccggcagtaacctcacgctgacggcgcaggtgctggcgaacaccggcagcggactggtacaggctgccaccctgctgctggatgtggtgaatactgtcaacggcggacgcgtacttgccaccggcagtgacgttaaaggaaccacgctgaataataccggtacgcttcagggtgcgactctggtgaattaccacacattcagcagcggtaccctgctgggaacctccgggcttggcgtcaagggcagttcactgctgcaaaatggtacagggcggctgtacagtgcaggcaacctgctgcttgacgctcaggacttcagtggtcaggggcaggtggtggccaccggtgatgtcacactgaaactgattgctgccctcacgaatcatggtaccctggccgcagggaaaaccctttccgtcacgtcgcaaaatgccatcaccaacggcggtgtcatgcagggtgatgccatggtgctcggtgccggagaggcattcaccaacaatggactgactgccggtaaaggcaacagtgttttcagcgcacagcgtcttttccttaacgcaccgggttcacttcagggcggtggcgatgtgagtctgaacagccggagtgatatcaccatcagtggttttaccggcacggcaggcagtctgacaatgaatgtggccggtaccctgctgaacagtgcgctgatttatgcggggaataacctgaagctgtttacagaccgtctgcataaccagcatggtgatatcctggccggcaacagtctgtgggtacagaaggatgcttccggcggtgcaaacacagagattatcaataattccgggaatattgagacgcatcagggcgatattgttgtaagaaccgggcatcttctgaaccagcgggagggattttctgccacaacaacaacccggactaacccctcatccattcagggaatgggaaatgctctggttgatattcccctttcccttcttcctgacggcagctatggctatttcacccgtgaagttgaaaatcagcacggtacgccctgcaacgggcacggggcatgcaatatcacaatggatacgctttattattacgcgccgtttgctgacagtgccacacagcgctttctcagcagccagaacatcacaacagtaaccggtgctgataatccggcaggccgcattgcgtcagggcgtaatctttctgctgaggctgaacgactggaaaaccgggcgtcatttatcctggcgaatggggatatcgcactctcgggcagagagttaagcaatcagagctggcagacggggacagagaatgaatatctggtataccgctacgacccgaaaacgttttacggtagctatgcaacaggctctctggataaactgcccctgctgtcaccggaatttgaaaacaataccatcagattttcactggatggccgggaaaaagattacacgcccggtaagacgtattattccgttattcaggcgggcggggatgttaagacccgttttaccagcagtatcaataacggaacaaccactgcacatgcaggtagtgtcagtccggtggtctctgcacctgtactgaatacgttaagtcagcagaccggcggagacagtctgacacagacagcgctgcagcagtatgagccggtggtggttggctctccgcaatggcacgatgaactggcaggtgccctgaaaaatattgccggaggttcgccactgaccggtcagaccggtatcagtgatgactggccactgccttccggcaacaatggatacctggttccgtccacggacccggacagtccgtatctgattacggtgaacccgaaactggatggtctcggacaggtggacagccatttgtttgccggactgtatgagcttcttggagcgaagccgggtcaggcgccacgtgaaacggctccgtcgtataccgatgaaaaacagtttctgggctcatcgtattttcttgaccgcctcgggctgaaaccggaaaaagattatcgtttcctgggggatgcggtctttgatacccggtatgtcagtaacgcggtgctgagccggacgggttcacgttatctcaacggactgggttcagacacggaacagatgcggtatctgatggataacgcggccagacaacagaaaggactgggattagagtttggtgtggcgctgacagctgaacagattgctcagcttgacggcagcatactgtggtgggagtcagtcaccatcaacggacaaacagtcatggtcccgaaactgtatctgtcgccggaagatatcaccctgcataacggcagcgttatcagcgggaacaacgtgcaacttgcgggcggcaatatcaccaacagcggcggcagcatcaacgcacagaacgacctctcgctcgacagttccggctatatcgacaacctgaatgcggggctgataagcgcgggcggtagcctggacctgagcgccatcggggatatcagcaatatcagctcagtcatcagcggtaaaaccgtacaactggaaagcgtgagtggcaacatcagcaatatcacccggcgtcagcaatggaatgcgggcagtgacagccaatatggtggtgtgcatctcagcggtacggacaccggtccggttgcgaccattaaaggcactgattcactttcgctggatgcagggaaaaacattgatattaccggggcaacggtctcgtccggtggagaccttggaatgtctgcgggtaatgatatcaacattgccgcaaacctgataagtgggagcaaaagtcagtccggtttctggcacactgatgacaacagttcatcatccaccacctcacagggcagcagcatcagcgccggcggtaacctggcgatggctgcaggccataatctggatgtcacggcatcctctgtttctgccgggcacagcgccctgctttcttgcaggtcacgacctagtcttgaatgcagtcagggaaaagcaaaaacaagtcgcaacggcaggtcagaaagtcatgaaagccacgcagctgtgtccacggtgacagcgggcgataacttcctccttgttgccggtcgtgatattgccagtcaggctgccggtatggctgcggaaaataacgtggtcatccggggcggacgtgatgtgaacctggtggcagagtctgccggcgcaggcgacagctatacgtcgaagaaaaagaaagagattaacgagacagtccgtcagcagggaacggaaatcgccagcggtggtgacaccaccgtcaacgcaggacgggatatcaccgctgttgcgtcatccgttaccgcaaccggcaatatcagcgtgaatgccggtcgtgatgttgccctgaccacggcgacagaaagtgactatcactatctggaaacgaagaaaaaaagcggaggttttctcagtaagaaaaccacccgcaccatcagtgaggacagtgccacccgtgaagcaggctccctgctgtcggggaaccgcgtgaccgttaacgccggtgataacctgacggtagagggttcggatgtggtggctgaccgggatgtgtcactggcggcgggtaaccatgttgatgttcttgctgccaccagtacagatacgtcctggcgctttaaggaaacgaagaaatccggtctgatgggtaccggcggtattggtttcaccattggcagcagtaagacaacgcacgaccggcgcgaggccgggacaacgcagagtcagagtgccagcaccatcggctccactgccggtaatgtcagtattaccgcgggcaaacaggctcatatcagcggttcggatgtgattgcgaaccgggatatcagcattaccggtgacagtgtggtggttgacccggggcatgaccgtcgtactgtggacgaaaaatttgagcagaagaaaagcgggctgacggttgccctttccggcacggtgggcagtgccatcaataatgcggttaccagtgcacaggagacgaaggagagcagtgacagccgtctgaaagccctgcaggccacaaagacagcgctgtctggtgtgcaggccggacaggctgcgacaatggcctccgcaaccggtgacccgaatgcgggagtcagcctgtcgctcaccacccagaaatcgaaatcacaacaacattctgaaagtgacacagtatccggcagtacgctgaatgccgggaataatctgtctgttgtcgcaaccggcaaaaacaggggcgataaccgcggagatattgtgattgcaggaagccagcttaaggccggtggtaacacaagcctggatgccgcgaatgatattctgttgagtggcgccgcaaacacacaaaaaacaacgggcaggaacagcagcagtggcggtggcgtgggtgtcagtatcggtgcaggtaaaggtgccggtatcagcgcctttgccagcgttaatgcggcaaaaggcagggagaaaggtaacggtactactaccgacaaaaccgtcaccatcaacagtggtcgggatacggtactgaacggtgctcaggtcaacggcaacaggattatcgccgatgtgggccacgacctgctgataagcagccagcaggacaccagtaagtacgacagtaaacagaccagcgtggctgccggcggcagttttacctttggctccatgaccggctcaggttacatcgctgcctcccgggataagatgaagagccgctttgactccgttgctgaacaaaccggaatgtttgcccgggtgatggtggcttcgacatcacagtgggtaaacatacccaactggatggtgcggtcattgcctcactgccacaccggagaaaaaccacctggataccggacgctgggtttagtgactttacaacgaagcgggattataaagtcaagtcacaggtggaatcagtctga

SEQ ID NO: 110 - DNA, 963 bp, Escherichia coli
atgatgttgaagaaaacgatatttatattaacgttattctctggcaacgtaattgctgcaactgtagaattaggttttgaaaatgagcaatataattatgcttatcgttctgcagatgtcttcatgccgtatattaagagtaatttcaaccctgttactgattctgctttgaatgtgtcactcacctatatgtatcaggatcaatatgggaaaaaacataaaaaaacatctgaggacagatttaaaaccaatcgcgatcgcatagagctctatcttaaaggttatactttaaataggggagcatattctttttctccttccgcaggtttccgttatgagtcatgggatgtaaactacgataatccgaaaaagcaggataagtggaaactggaactacgcttttatcctaatatgacttataaactcaatgaccagttaagcctatatatgaatggttttgttgcccctgtattttttaaaacacaacaagagtcgagaaaagataacaattatgtaaagggtaagttaggggcgaaacgttataacaacgattattatcaggaactccagattctgggtgtcagatataaatttaataatgataatacgctctgggcatcagtctataatgaaagaaaatataatcaacattcctcaaaatatgatcgctggcaattgcgtggaggctatgattttaaagttacagaggagtttgttttgagtccattcataagatatgacctctcttatagagaaaaaaacctcgaaagcacaagtaataatggtttatcaaaaaataataaagaaattcgaactggagccagcttttcctataaaattatcccttctgtaaaactggtaggagaaatatacaggcaaacaaccaacattgaaaactattatggagagcattctgaagacaaaaaccgcatgttctacaaacttggtataaacaaaacattt

SEQ ID NO: 111 - DNA, 1761 bp, Escherichia coli
atgcagcaccggcagaaaaacattctgacgaaaacgtcccttttatcccgtgcgttgtctgtcccctgttgtgatatgttccggcgcggctctccgtggatatgctatctctccctctccgttttttctggttgtttcatccccgcattttcgtctccggcagccatgctgtctccgggtgaccgcagtgcaattcagcagcaacagcagcagttgctggatgaaaaccagcgtcagcgtgatgcgctggagcgcccgctgaccatcacgccgtctccggaaacgtctgccggtactgaaggtccctgctttacggtgtcaagcattgttgtcagtggggccacccgactgacgtctgcagaaaccgacagactggtgccgtgggtgaatcagtgtctgaatatcacggggctgaccgcggtcacggatgccgtgacggacggctatatacgccggggatatatcaccagccgggcctttctgacagagcaggacctttcagggggcgtactgcacataacggtcatggaaggcaggctgcagcaaatccgggcggaaggcgctgaccttcctgcccgcaccctgaagatggttttcccgggaatggaggggaaggttctgaacctgcgggatattgagcaggggatggagcagattaatcgtctgcgtacggagccggtacagattgaaatatcgcccggtgaccgtgagggatggtcggtggtgacactgacggcattgccggaatggcctgtcacagggagcgtgggcatcgacaacagcgggcagaagagtaccggtacggggcagttaaatggtgtcctttcctttaataatcctctggggctggctgacaactggtttgtcagcgggggacggagcagtgacttttcggtgtcacatgatgcgaggaattttgccgccggtgtcagtctgccgtatggctataccctggtggattacacgtattcatggagtgactacctcagcaccattgataaccggggctggcggtggcgttccacgggagacctgcagactcaccggctgggactgtcgcatgtcctgttccgtaacggggacatgaagacagcactgaccggaggtctgcagcaccgcattattcacaattatctggatgatgttctgcttcagggcagcagccgtaaactcacttcattttctgtcgggctgaatcacacccacaagtttctggggggggtcggaacactgaatccggtattcacacgggggatgccctggttcggcgcagaaagcgaccacgggaaaaggggagacctgcccgtaaatcagttccggaaatggtcggtgagtgccagttttcagcgccccgtcacggacagggtgtggtggctgaccagcgcttatgcccagtggtcaccggaccgtcttcatggtgtggaacaactgagcctcgggggtgagagttcagtgcgtggctttaaggatcagtatatctccggtaataacggcggttatctgcggaatgagctgtcctggtctctgttctccctgccatatgtgggaactgtccgtgcagtggctgcactggacggcggctggctgcactctgacagcgatgacccgtactcgtccggcacgctgtggggtgctgctgccgggctcagcaccaccagtggccatgtttccggttcgttcactgccggactgcctctggtttacccggactggcttgcccctgaccatctcacggtttactggcgcgttgccgtcgcgttt

SEQ ID NO: 112 - DNA, 2220 bp, Escherichia coli
atgaataagcacacactattactgactgttctttttctgaatttgatttgtactcccgtttttgctcaaaactggcaggtggcgacgtttggtcagtctacggatctcaacttttcatcgctgatagattcggccaagatcggacggaataatgcctggcttgcaggaaacaataattttcttgaagctggaaaattttacactttaccaacagatttttttattgaaagccgtgggggaaaaattgctaactcccatgacggtatgaccgtcttttatactattgttccggttactcagacattccgactggaggctgatttgacattagaacagattggtccggaggtgaatggaaaatcaccagcgggacaggagggagctggattgtttgtcagagatattatcggtcctcagcgacaggaacctcagtcagctggaacagaagaatatccccaggcctctaatatattgatgaatgcctttattacacagaataaaaagaatgataacttagtacagattacttcaattgttcgtgaaggagtaataaaaacatggggtaatgaaggtattacaattaagaaacagccgatcattgagaatataaactttacgcaaaaaagaaatattcatatgacgatcgagcgactaccagagaagttcatcctgaccgcttttgataccgatcgtaaagaaaatcagtcatggcaattttctgattactcaggctttatgaatcaactggataataatagtttagctattggtttttttgccgcacgaaatgcgaaactaagggtgaaaaatgcatcatttaaaccgggcaagccactggttgattacaaacaattaacttcacgtcaattcagtcgtgtccggcataaagcccctgaactttttcttgcttcacctcaatccgttgtaagaaactcaacaactcttcaatttttggccaatcaggctggaatagtcagtattgataatgataagcagactaagcaggtgcaggcgggtgaactggtacagtttccagttactttgcaaaaaaaacataatgacttcaccgtcaactttaacgttgatgggaatatatcaaaaaaagctatacgcatagagcaggttaaatcaaacctgactgatccttatgagatttacgtatgtagtgattgtcgacagggggccagaggcagcaaaaatgaccctgtagatttacagacagccgtaaaatttgtcgcacccggcggtaatatataccttaacgatggtcaatatcatggaattaccttagatcgggaattaagtggaatacctggcaagtataaaacaatttctgccattaatccacataaagccatttttataaacaagacattcaatctggatgcaagttactggcatctaaaatccgtggtctttgacggcaatgtggataatggaaataataaaccagcatatttgcgtatagctggtagctataatattattgagcatgtgatagccagaaataatgatgatacgggaatttctatttcagcgaaagataaaaaccgttttttctggccagctcataacttagttttaaactcagattcatataataatcttgatttatccgggattaatgccgatggttttgctgcaaaattaggtgtcggaccgggaaacatttttcgaggatgcattgcacataataatgcagatgatggttgggacctatttaacaaaattgaagatggtccaaatgcatctgttactattgagaattctgtagcctatgaaaatggcctgccatacaataaagcggatatcctaaaagggagtattggcaatggcggtgaaggtcaacccagtaaatcacaagttattaattccattgctattaataataatatggatggattcactgataattttaatactgggtcattgatagttagaaataatatagcaatgaacaatgcacgctataattatattttaagaactaacccatataaattcccatcatctatcctttttgataataattattcaatcagagatgattgggaaaataaaataaaagacttcttaggtgatacagttaacagtgtgaattataaattgcttgtttcacatgaaacaggaccggtacaaaaagatttatttttcacacgagatgatagtggaaatattatctatcctgatttttttcttaatatcattaataaatttaat

SEQ ID NO: 113 - DNA, 408 bp, Escherichia coli
atgaaaacttttatcaaaactttactcgttgctgtaactattctgttctctgtcttcgctacggcgaaacaagtaaaactgccaaacaacatcaaatacgttaatactacagaggcgttttcctgtactgagattgacggtatgaattgccagacgaagaatccgtttaactataaagataacagctatgttttcgtgcttgaacgtggtggtgcctggtgttacgactacactgtctcggtacttaacctgaaaaccgggaaagcacagatgctcgaatacaaagacaaccagctgtgctcaggtagcaacaaaccgttcttcgaaatcaaaaatggcgtaccgacggtaggagtcatcgacacatccggaaaacctgtcgttgtggctctggacaaacttaaaacc

SEQ ID NO: 114 - DNA, 675 bp, Escherichia coli
atgcaattacctgtaaagttattaatgagccttatatctctggtcagcgttattgcacgtgccgggaaatataaaaattacatccgggatgaaataaaatactggcgatatacatcatacaaggggggggaatttccggaaggtttcactgatgagaaattttccagcgccatttacaacggaagaatatttacaatgaaacgtttacataccctgatgttatttctggcggttctgtttactggctttaacgtggaagcagcgagcgtgaaacaagcgctcagctgcgacccaaacgcccgggctgaacaacctggagcgtgtccaacaacgtacgagttgtacgaaggtgacgctgcctacaaagctgcgcttgacaaagcattaaaaccggtcggactgagcggcatgttcggtaaaggcgggtatatggatggccctggcggaaacgtaacgccagtaaccattaacggtacagtctggctccagggcgacggttgcaaagccaatacctgcggctgggactttatcgtaacactctataacccaaaaacccatgaagtcgttggctaccgctactttggtttagatgacccggcctacctggtttggttcggcgaaattggcgtgcatgaattcgcgtatctggtgaaaaactacgtagctgcggttaac

SEQ ID NO: 115 - DNA, 2163 bp, Escherichia coli
atgaaaactcaaataactttcgctgcgcttttgccagcattagcgtctttcataccgcttcatgctcatgcctcgtctacttctgaagatgaaatgattgtcacgggcaacaccgccgccgacaccaccgattctgccgccggtgccggtttcaaaacgaacgatatagatgtcggcccgctgggaacgaaatcctggatcgaaacaccatattccagcaccactgttactaaagagatgattgaaaatcagcaggcgcaaagcgtcagcgagatgctgaaatactctcccagtacgcaaatgcaggcgcgcggtggaatggatgtcgggcgtccgcaaagtcgggggatgcagggcagcgtggtggccaacagccgtctggacgggctgaatatcgtttcaacaaccgcgtttccggtggaaatgcttgagcgcatggatgtgcttaacagtttgaccggcgcgctgtacggcccggcgagcccagcagggcagtttaatttcgtggcgaagcgcccaaccgaagagacgctgcgtaaagtgacgctgggctatcaaagccgcagtgcgtttaccggccatgccgatctgggtggccattttgatgaaaacaaacggtttggctatcgcgtgaacctgcttgatcaggaaggggaaggcaatgtggatgacagcacgctgcgtcgcaaactcgtttccgttgcgctcgactggaatattcagccgggcactcagctacagctcgacgccagccattacgaatttatccagaaaggctatgtcggtagctttaactatgggccgaacgtcaaactgccgtctgcgccgaatccgaaggacaaaaatctggcgctcagcactgcgggcaacgacctcactaccgataccatcagcactcgcctgatccactactttaacgacgactggtccatgaacgctggcgtgggctggcagcaggctgaccgcgcgatgcgtagtgtttccagtaaaatactcaacaatcagggcgatatctctcgttcgatgaaggattccaccgctgccggacgttttcgcgtcctgagcaacaccgccgggctgaatggtcatattgataccggctctatcggccacgatctgtcactttctaccacgggatatgtctggtcgctttatagtgccaaaggaacaggttccagctatagctggggtacaacaaatatgtatcacccggatgcgatagatgagcagggcgatggcaaaatccgcaccggcgggccgcgataccgctccagcgtaaatactcagcagagcgttacgctcggcgatacggtgacatttacgccgcagtggtcggcaatgttctatctcagccagagctggctgcagactaaaaactacgataagcacggtaatcaaacgaaccaggttgatgaaaatggtttaagtccgaacgccgcgctgatgtataaaattacccctaacacaatggcctacgttagctatgccgattcgctggagcagggcggtaccgcaccgacggatgagagcgtaaaaaatgccggtcaaacgctaaacccgtatcgcagcaagcagtatgaagtggggctaaaatcggacatcggcgagatgaatctaggcgccgcgctgttccgactggaacgtccgtttgcctatcttgatacggataacgtgtataaagagcagggtaaccaggttaacaacggccttgagttaaccgctgccgggaatgtgtggcaggggctgaatatttacagcggcgtgaccttcctcgacccgaaactgaaagatacggcgaatgcctcaaccagcaataaacaggttgtcggcgtgccgaaagtgcaggccaatctgttggcggaatacagtttgccgtccataccggaatgggtttacagcgctaacgtccattatacgggcaaacgcgcggcgaacgataccaacacctcttacgccagcagctataccacatgggatttgggaacgcgttacaccacgaaagtgagcaacgtcccaaccactttccgcgtggtggtaaacaacgtgtttgataaacattactgggcttctatcttcccatcgggtaccgatggcgataacggttccccaagtgcgtttatcggcggcggccgcgaagtgcgtgcatccgtcaccttcgatttc

SEQ ID NO: 116 - DNA, 2007 bp, Escherichia coli
atgaaaaacataacgctgtggcagcgtttaagacaggtcagtatcagtaccagcttacgttgcgcatttctgatgggggcacttctgaccctgattgtcagtagtgtcagtctgtattcatggcatgaacaaagctcacaaattcgttactcgctggataagtattttccccgtattcactctgctttccttattgaagggaacctgaatctggtggtagaccagctaaatgaatttttgcaggctcccaacaccacggtgcgattgcaacttcgtacccagattattcagcatctcgacaccatagaacggcttagtaggggactgtcatcccgggaacgccaacaactgacggtcattttgcaggacagtcgatcactgttatccgagttggatcgtgcgctttacaacatgtttttactacgggaaaaggtgagtgagctatcagcgcggattgactggttacacgatgattttactaccgagcttaattctttagtgcaggatttcacctggcagcagggaacgctgctggatcaaatcgcctcccgacagggcgatacggcgcaatacctgaagcgatctcgtgaagtgcaaaatgaacagcagcaggtttataccctggcacgcattgaaaatcagattgttgacgatctgcgtgacagactcaatgagctcaaatcaggacgtgatgacgacatacaggtggaaactcatctccgttattttgaaaatctgaaaaaaacggcagatgaaaatatacgtatgctggatgactggcctggcaccattaccctgaggcagaccatcgatgaattgctggatatgggaatcgtaaaaaacaaaatgccggatacgatgcgtgaatatgtcgccgcccaaaaagccttagaggatgccagtcgcaccagggaagcgacacagggtcgcttcagaacgttactggaagcgcagcttggcagtactcatcaacaaatgcagatgtttaatcaacgaatggaacaaattgttcacgttagcggtgggctgatcctggtggcgacagcactggcgttactgcttgcatgggtattcaaccattattttatccgctcacggttggtgaaacgctttaccctactgaatcaggccgttgtgcaaattggtctgggaggcacggaaacgactattccagtttatgggaatgatgaactggggagaattgcaggattattacgccatactctcggccaactcaatgtgcaaaaacagcaacttgaacaagaaattaccgatcgtaaggtgatagaagccgatctgcgtgccacccaggacgaactgattcagacagcaaagttggcggtagtcgggcaaacgatgaccacgctggcccacgagatcaatcagccgctaaatgcgctgtcaatgtatctgtttacagcccgcagggccattgaacagacccagaaagaacaggccagcatgatgcttggtaaagccgaaggggtgattagtcgtattgacgccattatccgttcactacggcagtttacccggcgcgccgaactggaaacatcactccatgccgttgatttagcacagatgttcagtgcggcctgggaacttctggccatgcgtcatcgctctctgcaagctacgcttgttctgccgcaaggtacagccacagtttcaggtgatgaggtcagaacccagcaggtactggttaacgtactggcgaatgcgcttgatgtttgtgggcaaggcgctgtcattaccgttaactggcaaatgcagggtaaaacgctgaacgtattcattggcgataatggcccgggctggcctgaggcattgttgccttcgttattgaagccgtttaccaccagtaaagaagtaggactgggtattggtctttcaatttgtgtgtcgttgatggagcaaatgaaaggggaattgcggctggcatcaacgatgaccaggaatgcctgtgtggtactgcaattcagactaacggatgtggaagatgctaag

SEQ ID NO: 117 - DNA, 2259 bp, Escherichia coli
atgaacgttataaaactggctatcggctcaggcatattattgctcagctgcggtgcttactcacaatccatcagtgaaaaaactaattccgacaaaaaaggagcggcagaattcagtccgctcagcgtttctgtcgggaagacgaccagtgagcaggaagctctcgagaaaacaggcgcgaccagttcccggacaacggacaaaaacctgcaatcacttgacgcaacagtgcgtagtatgcctggtacttatactcaaatagatcctggtcagggagcaatcagtgtgaatattcgaggcatgagcggatttggtcgtgtaaacactatggtcgatggtattacccagagtttttacggaacctctacctccggaacaacgacgcatggttcaactaacaatatggctggcgtacttatagatcctaacttactggtagcagttgatgttacacgcggtgacagcagtggctctgaagggatcaacgcccttgccggtagtgcaaatatgcgtactattggcgttgacgatgtaatatttaacggtaatacatatggccttcgttcacgtttctctgtcggtagtaatgggctgggacgcagcggaatgatcgcccttggtggaaaaagcgacgcttttacggatacgggaagcattggcgttatggctgctgtgagcggcagttctgtgtactctaatttctcaaatggttctggaattaacagcaaagagtttggttatgataaatatatgaagcagaaccccaaatcccaactgtataaaatggatatcagaccagacgaatttaacagcttcgaactttccgctcgaacctatgaaaataaatttacacgtcgtgatataaccagtgacgactattacattaaatatcattacacccctttttctgaattaattgactttaacgtaacggccagtaccagtcgcggtaatcaaaagtatcgtgatggctcgctgtatactttctacaaaacctcagcgcaaaatcgttctgacgcgctggatatcaacaataccagccggttcactgtcgcggacaatgacctggagtttatgctgggcagcaaactgatgcgtacccgctatgaccggaccattcactcagcggcgggcgacccgaaagcgaatcaggaatcgatcgagaacaatccgttcgcaccctccggccagcaggatatttcagcgctgtataccgggctgaaggttacgcgcggcatctgggaggcagatttcaatctcaactacacacgtaacaggatcacagggtacaagcccgcctgcgattcacgcgttatctgcgtgccacagggtagctacgatattgacgataaagagggtggcttcaacccttcagttcagctttctgctcaggtaacaccatggcttcagccgttcattggctacagcaaatccatgcgcgccccgaacatccaggagatgttcttctctaattcaggaggcgcatccatgaacccattcctgaagcctgaacgtgcagaaacctggcaggcgggttttaacattgataccagagatttactggtcgaacaggatgccctgcgctttaaggctctggcgtaccgcagcaggatccagaactacatctacagcgagtcttatctggtttgttctggaggtcgtaaatgcagtctgcctgaggtgattggcaatggctgggagggcattagcgatgaatacagcgacaatatgtacatctacgttaactcggcaagcgacgttatcgcaaagggcttcgaactggagatggattatgatgcaggttttgcttttggccgactctctttcagccagcagcaaacagaccagccaacctccatcgccagcacccactttggcgcaggggatataaccgaactgcccagaaaatacatgacgctggatactggtgttcgcttcttcgataacgcgttgaccctgggcactatcataaaatacacaggcaaggctcgtcgcctgtcgcctgattttgagcaggacgaacataccggcgcaataatcaaacaggatttgccgcagatcccaacgattatcgatctctatggtacttacgagtacaaccgcaacctgacactgaaactttcggtacaaaacctgatgaacagagattattcggaggcgctgaataagctcaacatgatgccaggtcttggtgacgagacccacccagccaattccgcgcgtggcagaacatggatatttggcggggacattcgtttc

SEQ ID NO: 118 - DNA, 399 bp, Escherichia coli
atgtcttcgaaaacaaaatgctggctatggatgttactggtcatcctttctgaaacctctgcaacatccacacttaaaatgttcgataacagtgaggggatgacaaaaacgctgctgctggccctaatcgtcgtactgtattgcatttgttactactcgctttcacgggcagtaaaagatatccccgttggtctggcttacgccacatggtccggtactggcattttgatggtttcaacccttgggattttattttacggtcaacacccggataccgccgccattattggtatggtcatcatagccagcggtattatcattatgaatctgttctcaaaaatgggcagtgaagaggcggaagaaactccagttaccaacctcgataaaaaaatcgctaac

SEQ ID NO: 119 - DNA, 858 bp, Escherichia coli
atgtatataaaaaagcactggatagctttatccattctattaataccttgcattggaaacgctcaggaaattaaaattgatgaaagctggttacatcaaagcttgaatgtcattggtcgcacagactctcgctttggcccaagactgactaacgacctctaccctgaatatactgtagcaggaagaaaagactggtttgatttttatggttatgttgatctaccgaaattctttggcgtcggcagtcactatgatgttgggatctgggatgagggctcaccactatttacggaaatagaacctcggttttccattgacaaattgaccggattaaatcttgcgttcggcccatttaaagaatggttcattgcaaacaactatgtctatgatatgggtgacaaccagtcatcccggcaaagtacatggtatatggggcttggtacagatatcgacacgggtctaccaattaagctttctgccaatatatacgccaagtatcagtggcaaaactatggtgccgctaatgaaaatgaatgggacggatatcgattcaaaataaaatatagcatccctcttacaaatttattcggaggacgattggtatacaatagttttactaactttgattttggctccgatcttgcggacaagtcacacaataataaacgaaccagtaatgctattgcttcaagccatatcctttcccttctatatgaacactggaaatttgcatttacactacgttattttcacaacggtggacaatggaatgcgggagagaaggttaacttcggagatggtccatttgaattaaaaaatacaggatggggaacctatactactattggttatcaattt

SEQ ID NO: 120 - DNA, 516 bp, Escherichia coli
atgagaatcgcaccgcgtaccttctttgctatttccgccctggcgtttattgtcgcctccggatttagtttctggcggttgtcccctgctgaaaatacagggattatgagttgttcaacaaaaggcatcatgcgttttgagaatatggaaaaggagaacgttaacggtaatattcactttaactttggcagccagggtaaaggttcgatggtgctcgaaggctacacggactctgccgctggctggctgtacctgcaacgctatgtcaaatttacctataccagtaaacgtgtttccgccacggaacgccattaccgcatcagccagtgggaatccagcgcctcatcgatagatgaatcaccagatgtgatttttgactactttatgcgtgaaatgtctgacagccatgacgggctgttcctcaacgcccagaagctgaacgataaagcgattttgctcagttctattaattcaccgctttggatctgtacccttaaatctggcagcaaattagac

SEQ ID NO: 121 - DNA, 546 bp, Escherichia coli
atgaaaataaaagttatagcattggctacatttgtttctgctgtgtttgctggttcagctatggcctatgacggaacaattacgtttaccggtaaagttgtagctcagacctgcacagttaatacaagcgacaaagacttagcagtaactttacccactgttgccacttcatctctaaaagacaatgctgctacgtcagggctgacaccctttgccattcgtttaactggttgtgcaactggtatgaatagtgctcagaatgttaaagcgtactttgagccttcaagtaacattgacttagctacacataatttaaaaaatactgctactccaactaaagcggataatgtacagattcagttgctaaatagtaatggaacttcaactattcttttgggggaagcggataatgggcaagatgtccagtctgagacaatcggatctgatggaagtgccacattgcgttatatggcccagtattatgcaacaggacaatctaccgcaggggatgtaaaagcgacggtccattataccattgcctacgaa

SEQ ID NO: 122 - DNA, 1077 bp, Escherichia coli
atgaaaagaatctttttcataccattgtttttaattttactccctaagctggcggtagcgggtccggatgattatgtgccttcgcagatagcggttaatacatccacattgccaggtgttgtgattggtcctgctgatgcccatacctatccccgggtgataggagagctggcgggaacaagtaaccagtatgtttttaatggcggtgccatcgctctgatgcgtggaaagtttacacccgcactgcctaaaattggaagtattacggtatactttccatcaaggaaacagcgtgattcatctgattttgatatctatgatattggtgtatccggactgggtattattattggcatggcgggctattggcccgcaacgcctctggtccccataaatagctcaggtatatatattgaccctgtaggtgccaatacaaaccccaatacttataacggtgcgacagcaagcttcggagctcgtttgtttgttgcttttgtcgcaacgggaagattacccaatggatatataacaatacccaccaggcagcttggtactattttgttggaagcaaaacgtacaagtttaaataataaaggactgacagcacctgttatgttaaatggtgggcgcattcaggtacagagtcagacatgtaccatggggcaaaaaaactatgtggtgccattaaataccgtatatcaatcacagttcacatctttgtataaagaaatacagggaggtaaaattgacatacacctacaatgcccggatggaattgatgtttatgctacattgacagatgcatcacagccagtgaacagaacagatatattgaccttaagcagtgaatccactgcaaaaggatttggcatcaggctatataaagacagtgatgtaactgccatcagctatggtgaagactcccctgtgaaaggaaatggcagtcaatggcacttctccgattacaggggagaggtaaatccacatatcaatttaagagccaattatataaaaattgctgatgcaactacacctggaagtgtgaaggctattgcaactattactttctcatatcaa

SEQ ID NO: 123 - DNA, 2532 bp, Escherichia coli
atgaacgctaataatctgtcatgcctgatttactgtcgttgttctcttctgctttttgctgcattagggttaacagtaacaaaccattcatttgctgctgaagaggctgagtttgattctgagtttttgcatttggataaagggataaatgctattgatatccgccgctttagtcatggtaaccctgtgcctgagggcaggtattattctgatatttatgttaataatgtatggaaggggaaggctgatttgcagtatttacgtactgccaataccggtgctccgacgttatgcctgacgcctgagctgctttcattgattgatttagtcaaagatactatgtcgggaaacacctcctgctttccggcgtcaacagggctttcttcagccagaattaattttgacttatcgactttaaggttgaatatcgaaatccctcaggcactgctgaatacacgtccaagaggatatatttcccctgctcagtggcaaagtggtgttcctgcagcatttataaactatgatgctaactattaccagtatagctcttccgggacgagtaacgaacagacttatctgggattaaaagctggattcaatttgtggggatgggctttgcgccaccgtggcagtgagagctggaataatagctatcctgccggatatcagaatatagaaacaagtataatgcatgaccttgccccattgagagcacaattcacattaggggatttttatacgaatggtgagctaatggatagcctcagtttgcggggagtcaggttagcatcggatgaacgaatgttacccggctctttacgtggctatgctcctgctgtccggggaatagctaacagtaatgctaaagtaaccatttatcaaaatgctcatatcctctatgaaacgacggtgccagccggaccatttgtcatcaatgatttatatcccagtggatatgctggtgaccttctcgttaagataacagagtctaatggccagacacgaatgttcacggttccttttgcggccgttgctcaactcattcgtcccggatttagtcgctggcaaatgtcagtgggaaagtatcgttatgcgaataaaacatataatgatttaatagcacaaggcacctatcaatacggcctgacgaatgatattactttaaacagtggtcttaccacagcttcaggatatacagcggggttagctggcctggcctttaatacccctctgggtgctatagcatctgacattacattgtccagaacagcattcaggtattccggtgtaacgcgtaaaggttatagtctgcactcaagttatagcatcaatattccagcctcaaacacaaatataactctggcggcttatcgttattcatcaaaagatttttatcatctgaaggatgcgctatcagctaatcacaacgcgtttattgatgatgtttctgtaaaaagtacagcgttttatcgtcccaggaatcaattccagatttcaatcaaccaggaattaggtgaaaaatggggtgggatgtatttaacaggaacaacctataattactggggacataaaggaagtcgtaatgaataccagattgggtacagcaacttctggaaacaactcggctatcaaattggattgtctcagtcaagagataatgagcaacaacgccgtgatgacagattttatattaattttactctccctctgggaggaagtgttcaaagcccggtgttttccactgttttaaattatagcaaagaagagaaaaatagtattcagacatcaattagtggtactggcggggaggataatcagttctcttatggtatttcaggaaacagccaggaaaacgggccttccggttatgcaatgaatgggggttatcgttcaccttatgtaaatataaccacaacagtcgggcatgatactcagaataataatcaaaggtcatttggtgcgtcgggagcggtggtcgcacacccctatggagtgacattgagtaatgacctgagtgatacttttgccattatccatgctgaaggagctcagggggctgtcatcaataatgcctctggtagtcgtctggatttttggggaaatggtgttgttccttatgttacaccctatgagaaaaatcaaattagcatcgatccctccaatttagatttgaatgttgaattatcggcgacggagcaggaaatcattcctcgtgctaatagcgccacgttagtgaaatttgacactaaaacaggaagaagtctgttatttgatattcgtatgtctactggcaatccccctccaatggcttctgaagttctggatgaacatggacagttggccggatatgtcgctcaggccgggaaggtatttaccaggggactccctgaaaaaggtcatctcagcgttgtatggggaccagataataaagacagatgttcatttgtatatcatgttgcacacaataaagatgatatgcaatctcagctcgttcctgttctgtgtatacagcaccctaatcaggaaaaaaca

SEQ ID NO: 124 - DNA, 831 bp, Escherichia coli
atggtaaaatgtcatactctgattaaccgtagaaataaatgtctgctgattgtttttatagtccttattggatggattatattcagacctaaagcatatacttattcactaaatgataaagaaaaagagatgctcataatgttatcacaacatcctgaaactcggtactttggattttattccatagaacttccggctgattacaaaccaacaggaatggttatgttcatacaaggatcggcgatgatccctgtagaaacaaagctacaatattatcctccttttctgcaatatatgacacgatatgaggcagaactaaaaaacacctcagcattagatccactggatacgccttatttgaagcaagttcacccactaagtccacctatgaatggagtcatttttgaacgaatgaaagcgaaatacaccccagattttgcacgagtattggatgcatggaaatgggaaaatggcgttacgttttcagtaaaaatagaagctaaagatggtagagcaacccgctatgatggaattagtaagattgccgaatacagttatggatataatattccagaaaaaaaagtacagttacttactattctttcaggactacaacctcgtgcagataaccaacccccatcagaaaataaattggcgatacaatatgcacaggttgacgcttcactacttggagagtatgaattatctgtagattataaaaatagcaataatattaaaataagtttgcagacggataataatagttatattgactcattattagatataagatatccgagtaatggaaacagagcatggtataactctata

SEQ ID NO: 125 - DNA, 1098 bp, Escherichia coli
atgctacctgagcctgtttatcgacgctggattatattattaatatctatgttaacagttggtactctgtttattttatcggtctggaattctgcgacatactgggatatttttatttatggcgttctgccaatgctgtttctttggctatgtttgtttggtattgcgctgaacaaatatgaacaatccgttgcagcctgtataagttgggagtctgaaagacaacaagttaaacaactctggcaacactggagccaaaaacaactggcaatagttgggaatgttctttttacaccggaagaaaaaggcatgagtgttttactggggccacaggaagagatccctgcatatcctaaaaaggcacgaccgttattctctgcatcccgttattctctttcgtctatattccatgatattcaccagcaactgacacaacaatttcctgattatcgtcattatctacatactatctacgtattacagcctgagaaatggcgtggagaaaccgtgagacaggctattttccatcaatgggacttagtacctgaacggaccaatactcttaatcaaatccagtctctttatgatgaaagatttgacggtctaattctggttgtttgtttacaaaactggccggagaataaacctgaagatacgagtgaactggtatcagcacagcttatctcctcatcgtcatttgtacggcagcaccagatacccgttattgctggtctggggcgtgtaatgccattagaacccgaggagttggagcataatctggatgtgttatttgaatataaccaattggataacaaacaactacagcatgtctgggtctctggtttagatgagggaacgatagaaaaccttatgcagtatgctgaacaacatcaatggtcacttcctaaaaaacggcccctacacatgattgatcattcctttggccctacaggagagtttatttttcctgtctctctggcaatgctgtcagaggctgccaaagaaactgaacaaaatcatttaattatctatcagtcagcacagtatgctcagaaaaagagcctttgcctgattacccggaagctttatttaaggaca

SEQ ID NO: 126 - DNA, 780 bp, Escherichia coli
atgttgaacagaaaactaaatatacggctacgtcattccctgaacagtcactgcataccttccatcattatcaataacaccgtacgttcatttcagaggtcagtcatgaataccagagctctttttcccctgctgttcactgtggcatcattctccgcctccgccggcaactgggctgtcaaaaacggctggtgtcagaccatgacggaagatggtcaggcgctggtaatgctgaaaaatggcacgattggtattaccggcctgatgcagggatgcccgaatggtgtacagacgctcctgggcagccgtatcagtattaacggtaacctgatccccacatcacaaatgtgtaatcagcagacgggattcagggctgttgaggtggaaatcggacaggcgccggaaatggtcaaaaaagccgttcactccatagcagagcgtgatgtgtccgttttacaggcatttggtgtacgaatggaattcacccgcggtgatatgctgaaggtctgtccgaaatttgtcacatcacttgccggtttttccccgaaacagacgaccactattaataaagattccgtcctgcaggctgcccggcaggcatacgcccgggaatatgacgaggaaacaacagaaaccgctgattttggctcttacgaagtaaaaggcaataaggttgagtttgaagtattcaatcctgaagaccgtgcgtacgacaaagtgaccgtcacggttggtgctgacggtaatgccaccggcgccagcgttgaatttatcggaaaa

SEQ ID NO: 127 - DNA, 1155 bp, Escherichia coli
gtggtaattatcaatagcacgatactgagcggcgcaggcgctatcccttccctgacgtcgctcttacccgacatcagaaaaatgctgctggtcactgaccgtaatattgcgcagctcgacggtgtgcagcagattcgcgccttactggaaaagcactgcccgcaggttaacgttatcgataatgtgcccgcagagcccacgcatcatgatgtgcgccagctaatggatgcccctggcgatgcctcttttgatgtggtggtcgggatcggcggtggcagcgtgttggatgtggcgaagctgctatcggtgctttgccatccacaatcaccggggctggatgcgctgcttgcgggtgaaaaaccgactcagcgggtgcaatcatggttgattcctacaaccgccggaaccggctcagaagccacgccgaatgcgattctggcaatccctgagcaaagcacgaaggtgggtattatttcccaggtgctgttaccagactatgtggcgcttttcccggaactgaccaccagcatgcccgcgcatattgcggcgtccacgggcattgatgctctttgccacttactggagtgttttaccgcgaccgtggcaaatccggtcagcgataacgcggcgctgactgggttaagtaaacttttccggcacattcaacccgccgtgaacgatcctcaggatctgcgcgcaaaactggaaatgctgtgggcgtcttactatggcggcgtagcgataacccatgcgggcacgcatctcgttcatgcgctctcctacccgttaggtggcaaatatcatctgccgcatggcgtcgcgaatgccatcttgctggcgccgtgcatggcgtttgttcgcccctgggcggtcgagaaatttgcccgggtctgggattgcattcccgatgcggaaaccgccctgagcgcggaagaaaaatctcatgccctggtgacctggttacaggcattagtcaatcaactcaagctacccaacaatctcgcggctctcggcgtaccgccagaggatattgcctctctgagcgaggcggcactgaacgtgaagcgccttatgaacaatgtgccgtgccaaattgatctacaggacgtacaggccatttaccaaacactgtttccgcaacatccatttaaggag

SEQ ID NO: 128 - DNA, 315 bp, Escherichia coli
atgaatatcagaaaactgttttgtccgggaaacaccccccggattttattgtttttattcttttttgttgtttctgcaataaccacaattgcatgcggatacactgagaagaatgcaacaggaaatgtgctgcttctgtttctccttctgctccttgcacacagaaataccctcacatccattacagcgctgttatttctgttctgttgtgcactgtatgcgcctgccggtatgacgtacggtaaaatcaacaacagttttattgtcgcgttgttgcagaccacaactgatgaggcagcggagtttaccgggatg

SEQ ID NO: 129 - DNA, 441 bp, Escherichia coli
atgaatattcaggcaataaaagaaatggtaaatttaatttgtagttttttatttatattctttctgtcctcggcttttgtttcttttgggtgttatgctatttatgaattgtttttatggaatgatattattgtatatagctggggatatatattaattgtctttttacctttcacattatatgtaatgtcgtttgagattttgttttttgctattagtgggcgacgattgtctaaagtaacaatggtgcgcctttggttgataattaaaattattattgctttctctatttgcgcagtgttgattttttcttcaatttacaaaaaagaattattatctagaaattatattgcttgtagtggtatcccgtctgggtggatgccgggtctggcaacgaaatacgttaaagaaaaatcattatgcgaaaaaaatggcaataat

SEQ ID NO: 130 - DNA, 534 bp, Escherichia coli
atgtttcctattcgttttaaacgtccggcgttgctctgtatggcgatgctgacggttgttctgagtggctgcgggctgattcagaaagtggtggatgaatcgaaaagcgtggcctcagccgttttctacaaacaaatcaaaatactgcatctcgatttcttctcccgcagcgccctgaatacggatgcggaagatacgccgctttccacgatggtgcatgtctggcaactgaaaacccgcgaagattttgacaaggcggattacgacaccctgtttatgcaggaagagaagacgctggagaaggacgtactggcaaaacacaccgtctgggtaaaaccggaaggcacggcatccctgaatgtgccgctggataaagagacgcagtttgtcgccattattgggcagttttatcaccctgatgaaaaaagcgacagctggcgtctggtgatcaaaagggacgaactggaggccgacaagccgcgctcgattgaactgatgagaagcgacctgcgactgctgcctctcaaggataaa

SEQ ID NO: 131 - DNA, 627 bp, Escherichia coli
atgttcttaaaaagaaaatggtattacgcagtgacgacatctgtcgtcattactttgtgtggtggaggatattatatgtacaggcaagaatatcagatggttgtcactgtaccaactgctgacgcgaacgatcccaactggccaaataaaaggatacagtttgataccagcgaatggctacagcaacttcaatatattaaaatagatgatcattatatattgaatactcaatatactccaattgctaatttggatgactttggtattacattaaaattacagaacgcattaaatgggtcggataaaagacttcctgcactatatggccttgctgagatggatgctcagaaatttaaagacctgatgcgcggtaaaattaaatgtgaatatctgaggacgacatttgatgcggaaacattaaagcctgtcaatgattatttccttatttcttttacttataaagataagtggtatgaatttgagacagaaagaaaaatatctaaaacaagtgatgatgggtattttttgtgggcatttgataatactgtccacgaagcaggctattggcataacacagatccggctgcgtattcctatagagattaccagaatggtaaggctgtgaaa

SEQ ID NO: 132 - DNA, 1272 bp, Escherichia coli
atggatatttggcggggacattcgtttctgatgacaatttccgctaggttcagacaatacgttttctctcttatgtcaattttattgcaggaacgaaaaatgaatattttcactttatccaaagcaccgctatacctgttaatttcactatttttacccacgatggccatggctatcgatccacctgaacgcgaactttcgcgatttgccctgaaaacgaattaccttcagtcccctgatgaaggcgtctatgaactggcgtttgataatgccagtaaaaaggtgtttgcagcagtcaccgatcgtgtaaatcgtgaagccaataaaggctatctgtattcgtttaattcagattcgctgaaagtcgaaaataaatacacgatgccataccgggcattttcgctggcgataaatcaggataaacatcagctctatatcggacacacccagtcagcgtccctgcgtatcagtatgtttgacaccccaaccggcaaactggtaagaaccagcgacaggttaagttttaaagcggcaaacgctgcagattcgcgttttgagcattttcgccatatggtttacagccaggattccgataccctgtttgtgagttatagcaatatgctgaaaacggccgagggcatgaagcctctgcataagctgttaatgctcgacgggacgacgcttgccttaaaaggcgaggttaaggatgcttacaaaggtacagcgtatggtctgacgatggatgaaaaaacacagaaaatctacgttggcggaagagattacatcaacgaaattgatgcgaaaaatcagacgctgctgcgtaccatcccgttgaaagatccgagaccacaaatcacaagtgtgcagaatctggcggtggactccgcttctgaccgtgcctttgtggtggtattcgaccatgacgatcgttccggtacaaaagatggactctatatttttgacttacgcgacggtaaacagcttggctatgtgcacacaggagccggagctaacgcggtgaaatacaatccgaaatataacgaactgtatgtcaccaacttcactagcggcaccatcagcgtagtggatgccaccaaatacagcatcacccgtgaatttaacatgccggtctacccaaaccagatggtgttgtcggacgatatggataccctttacattggcatcaaagaaggctttaaccgcgattgggatcctgatgtgtttgtggaaggagctaaagaacgtattctgagcattgatttgaaaaagtcg

SEQ ID NO: 133 - protein, 163 aa, Escherichia coli
MAIPAYLWLKDDGGADIKGSVDVQGREGSIEVVALDHDVYIPTDNNTGKLTGTRTHKPFTFTKEIDASSPYLYKAVTTGQTLKTAEFKFYRINDAGQEVEYFNITLDNVKLVRVAPLMHDIKDPSREKHNHLERIEFRYEKITWTYKDGNIIHSDSWNERPSA

SEQ ID NO: 134 - protein, 550 aa, Escherichia coli
VRNTLKQAIVLWGMVLLLVLWSVFISPSGVLRWAGAAAIVLAVAALLIYRRRQAWTEMTGDAGLSSLPPETYRQPVVLVCGGLSAHLSTDSPVRQVSEGLYLHVPDEEQLVAQVERLLTLRPAWASQLAVAYTIMPGIHRDVAVLAGRLRRFAHSMATVRRRAGVNVPWLLWSGLSGSPLPERASSPWFICTGGEVQVATSTETTMPAQWIAQSGVQERSQRLCYLLKAESLMQWLNLNVLTALNGPEAKCPPLAMTVGLVPSLPAVDNNLWQLWITARTGLTPDIADTGTDDALPFPDALLRQLPRQSGFTPLRRACVTMLGVTTVAGIAALCLSATANRQLLRQVGDDLHRFYAVPVEEFITKARHLSVLKDDATMLDGYYREGEPLRLGLGLYPGERIRQPVLRAIRDWRPPEQKMEVTASLQVQTVRLDSMSLFDVGQARLKDGSTKVLVDALVNIRAKPGWLILVAGYTDATGDEKSNQQLSLRRAEAVRNWMLQTSDIPATCFAVQGLGESQPAATNDTPQGRAVNRRVEISLVPRSDACQDVK

SEQ ID NO: 135 - protein, 194 aa, Escherichia coli
MIKSTFWRALALTATLILTGCSHSQPEQEGRPQAWLQPGTLITLPAPGISPAVNSQQLLTGSFNGKTQSLLVMLNAEDQKITLAGLSSVGIRLFLVTYDAKGLRAEQSIVVPQLPPASQVLADVMLSHWPISAWQPQLPTGWTLRDNGDKRELRNASGKLVTEITYLNRQGKRVPISIEQHVFKYHITIQYLGD

SEQ ID NO: 136 - protein, 129 aa, Escherichia coli
MKRYIKWFAITIFISMLSACVRTAPVQQISTTVSVGHTQEQVKNAILKAGAQRKWIMTQVSPGVIKARYQTRNHVAEVRITYTATYYNIKYDSSLNLQASDGKIHKNYNRWVRNLDKDIQVNLSTGATL

SEQ ID NO: 137 - protein, 415 aa, Escherichia coli
MKRKHLLLLLLFSFSTNSAPLYSLIREAVMHDPIVMEARAELTSAQSRIEQASSAHWPVVTATGSKLLSQSHRYSYDYDTEDILPGIRGEVNIFASGAIEADVRRSESEAEYYHYKMEETKEETIHSFVSLYLDALREKQSIAVLEQSLSRHNAILNDLNTISIHDTGRESELVQAEARRLMVRQQINSRSRVLKTTLGKLSTWTKNPVTEADLENPFSRMTEAKLLTDFTQAPQKGNPSWLASQADVESKKAALKAQELARYPRVDLTGSVTRDDQQIGVNLSWDLFNRNASYGVTEKAAQIVAATGRLDSVARMIDETGRLSLITVRQSRGEMETLRRQEQASARVVDFYRLQFQVARKTLIELLNAENELYSVGLSRVQTEDQMLHGMLDYLYSQGMLLKWSGVNLSGEEEK

SEQ ID NO: 138 - protein, 201 aa, Escherichia coli
MKFLPLLALLISPFVSALTLDDLQQRFTEQPVIRAHFDQTRTIKDLPQPLRSQGQMLIARDQGLLWDQTSPFPMQLLLDDKRMVQVINGQPPQIITAENNPQMFQFNHLLRALFQADRKVLEQNFRVEFADKGEGRWTLRLTPTTTPLDKIFNTIDLAGKTYLESIQLNDKQGDRTDIALTQHQLTPAQLTDDEHQRFAAQ

SEQ ID NO: 139 - protein, 770 aa, Escherichia coli
MENFFMKNSKVFYRSALATAIVMALSAPAFATDSTVSTDPVTLNTEKTTLDQDVVINGDNKITAVTIETSDSDKDLNVTFGGHDITAASTVNQDFVEGVKVSGNKNVVINATDSTITAQGEGTYVRTAMVIDSTGDVVVNGGNFVAKNEKGSATGISLEATTGNNLTLNGTTINAQGNKSYSNGSTAIFAQKGNLLQGFDGDATDNITLADSNIINGGIETIVTAGNKTGIHTVNLNIKDGSVIGAANNKQTIYASASAQGAGSATQNLNLSVADSTIYSDVLALSESENSASTTTNVNMNVARSYWEGNAYTFNSGDKAGSDLDINLSDSSVWKGKVSGAGDASVSLQNGSVWNVTGSSTVDALAVKDSTVNITKATVNTGTFASQNGTLIVDASSENTLDISGKASGDLRVYSAGSLDLINEQTAFISTGKDSTLKATGTTEGGLYQYDLTQGADGNFYFVKNTHKASNASSVIQAMAAAPANVANLQADTLSARQDAVRLSENDKGGVWIQYFGGKQKHTTAGNASYDLDVNGVMLGGDTRFMTEDGSWLAGVAMSSAKGDMTTMQSKGDTEGYSFHAYLSRQYNNGIFIDTAAQFGHYSNTADVRLMNGGGTIKADFNTNGFGAMVKGGYTWKDGNGLFIQPYAKLSALTLEGVDYQLNGVDVHSDSYNSVLGEAGTRVGYDFAVGNATVKPYLNLAALNEFSDGNKVRLGDESVNASIDGAAFRVGAGVQADITKNMGAYASLDYTKGDDIENPLQGVVGINVTW

SEQ ID NO: 140 - protein, 660 aa, Escherichia coli
MSRPQFTSLRLSLLALAVSATLPTFAFATETMTVTATGNARSSFEAPMMVSVIDTSAPENQTATSATDLLRHVPGITLDGTGRTNGQDVNMRGYDHRGVLVLVDGVRQGTDTGHLNGTFLDPALIKRVEIVRGPSALLYGSGALGGVISYDTVDAKDLLQEGQSSGFRVFGTGGTGDHSLGLGASAFGRTENLDGIVAWSSRDRGDLRQSNGETAPNDESINNMLAKGTWQIDSAQSLSGLVRYYNNDAREPKNPQTVEASDSSNPMVDRSTIQRDAQLSYKLAPQGNDWLNADAKIYWSEVRINAQNTGSSGEYREQITKGARLENRSTLFADSFASHLLTYGGEYYRQEQHPGGATTGFPQAKIDFSSGWLQDEITLRDLPITLLGGTRYDSYRGSSDGYKDVDADKWSSRAGMTINPTNWLMLFGSYAQAFRAPTMGEMYNDSKHFSIGRFYTNYWVPNPNLRPETNETQEYGFGLRFDDLMLSNDALEFKASYFDTKAKDYISTTVDFAAATTMSYNVPNAKIWGWDVMTKYTTDLFSLDVAYNRTRGKDTDTGEYISSINPDTVTSTLNIPIAHSGFSVGWVGTFADRSTHISSSYSKQPGYGVNDFYVSYQGQQALKGMTTTLVLGNAFDKEYWSPQGIPQDGRNGKIFVSYQW

SEQ ID NO: 141 - protein, 719 aa, Escherichia coli
MRDEMLYNIPCRIYILSTLSLCISGIVSTATATSSETKISNEETLVVTTNRSASNLWESPATIQVIDQQTLQNSTNASIADNLQDIPGVEITDNSLAGRKQIRIRGEASSRVLILIDGQEVTYQRAGDNYGVGLLIDESALERVEVVKGPYSVLYGSQAIGGIVNFITKKGGDKLASGVVKAVYNSATAGWEESIAVQGSIGGFDYRINGSYSDQGNRDTPDGRLPNTNYRNNSQGVWLGYNSGNHRFGLSLDRYRLATQTYYEDPDGSYEAFSVKIPKLEREKVGVFYDTDVDGDYLKKIHFDAYEQTIQRQFANEVKTTQPVPSPMIQALTVHNKTDTHDKQYTQAVTLQSHFSLPANNELVTGAQYKQDRVSQRSGGMTSSKSLTGFINKETRTRSYYESEQSTVSLFAQNDWRFADHWTWTMGVRQYWLSSKLTRGDGVSYTAGIISDTSLARESASDHEMVTSTSLRYSGFDNLELRAAFAQGYVFPTLSQLFMQTSAGGSVTYGNPDLKAEHSNNFELGARYNGNTWLIDSAVYYSEAKDYIASLICDGSIVCNGNTNSSRSSYYYYDNIDRAKTWGLEISAEYNGWVFSPYISGNLIRRQYETSTLKTTNTGEPAINGRIGLKHTLVMGQANIISDVFIRAASSAKDDSNGTETNVPGWATLNFAVNTEFGNEDQSRINLALNNLTDKRYRTAHETIPAAGFNAAIGFVWNF

SEQ ID NO: 142 - protein, 199 aa, Escherichia coli
MRKVCAVILSAAICLSVSGAPAWASEHQSTLSAGYLHARTNAPGSDNLNGINVKYRYEFTDALGLITSFSYANAEDEQKTHYSDTRWHEDSVRNRWFSVMAGPSVRVNEWFSAYSMAGVAYSRVSTFSGDYLRVTDNKGKTHDVLTGSDDGRHSNTSLAWGAGVQFNPTESVTIDLAYEGSGSGDWRTDAFIVGIGYRF

SEQ ID NO: 143 - protein, 456 aa, Escherichia coli
MKKSTLSLAIGLLLACSTGMAKTQHLTLEQRLEAAEMRAAKAEGQVKQLQTQQAAEIREIKTAQGNTPVNGQSTTESEKKNATPPNLLLSGYGDLKIYGDVEFNMDAESNHGLLAMTNADVNSDPTNEWNLNGRILLGFDGMRKLDNGYFAGFSAQPLGDMHGSVNIDDAVFFFGKENDWKVKVGRFEAYDMFPLNQDTFVEHSGNTANDLYDDGSGYIYMMKEGRGRSNAGGNFLVSKQLDNWYFELNTLLEDGTSLYNDGNYHGRDMEQQKNVAYLRPVIAWSPTEEFTVSAAMEANVVNNAYGYTDSKGNFVDQSDRTGYGMSMTWNGLKTDPENGIVVNLNTAYLDANNEKDFTAGINALWKRFELGYIYAHNKIDEFSGVVCDNDCWIDDEGTYNIHTIHASYQFANVMDMENFNIYLGTYYSILDSDGDKIHGDDSDDRYGARVRFKYFF

SEQ ID NO: 144 - protein, 174 aa, Escherichia coli
MNGKAFLACVLMSVVLTGCETAKKISQVIRNPDIQVGKLMDQSTELTVTLLTEPDSNLTADGEAAPVDVQLVYLSDDSKFHAADYDQVATTALPDVLGKNYIDHQDFNLLPDTVKTLPPIKLDEKTGYIGVIAYFSDDQATEWKQIESVESIGHHYRLLVHIRASAIEMKKEEN

SEQ ID NO: 145 - protein, 1144 aa, Escherichia coli
LTLAWIFLLVWIWWQGPKWTLYEQHWLAPLANRWLATAVWGLIALVWLTWRVMKRLQKLEKQQKQQREEEKDPLTVELHRQQQYLDHWLLRLRRHLDNRRYLWQLPWYMVIGPAGSGKSTLLREGFPSDIVYTPESIRGVEYHPLITPRVGNQAVIFDVDGVLTTPGGDDLLRRRLREHWLGWLMQTRARQPLNGLILTLDLPDLLTADKSRRETLVQNLRQQLQEIRQSLHCRLPVYVVLTRLDLLNGFAALFHSLDKKDRDAILGVTFTRRAHESDGWRSELGAFWQTWVQQVNLALSDLVLAQTGAAPRSAVFSFSRQMQGTGEIVTALLAALLDGENMDVMLRGVWLTSSLQRGQVDDIFTQSAARQYGLGNSSLATWPLVETTPYFTRRLFPEVLLAEPNLAGENSVWLNSSRRRLTAFSTCGAALAALMVGSWHHYYNQNWQSGVNVLAQAKAFMDVPPPQGTDEFGNLQLPLLNPVRDATLAYGDYRDHGFLADMGLYQGARVGPYVEQTYIQLLEQRYLPSLMNGLIRDLNIAPPESEEKLAVLRVVRMMEDKSGRNNEAVKQYMARRWSNEFHGQRDIQAQLMVHLDYALEHTDWHAQRQSSDSDAVSRWTPYDKPIINAQQELSKLPIYQRVYQTLRTKALSVLPADLNLRDQVGPTFDNVFVAGNDEKLVIPQFLTRYGLQSYFVKQREGLVELTALDSWVLNLTQSVAYSEADREEIQRHITEQYISDYTATWRAGMDNLNVRDYEAMSALTDALEQIISGDQPFQRALTALRDNTHALTLSGKLDDKAREAAINEMDYRLLSRLGHEFAPENSALEEQKDKASTLQAVYQQLTELHRYLLAIQNSPVPGKSALKAVQLRLDQNSSDPIFATRQMAKTLPAPLNRWVGKLADQAWHVVMVEAVRYMEVDWRDNVVKPFNEQLADNYPFNPRATQDASLDSFERFFKPDGILDNFYKNNLRLFLENDLTFGDDGRVLIREDIRQQLDTAQKIRDIFFSQQNGLGAQFAVETVSLSGNKRRSVLNLDGQLVDYSQGRNYTAHLVWPNNMREGNESKLTLIGTSGRAPRSIAFSGPWAQFRLFGAGQLTNVTSDTFNVRFNVDGGAMVYQVHVDTEDNPFTGGLFSLFRLPDTLY

SEQ ID NO: 146 - DNA, 489 bp, Escherichia coli
atggctattcctgcttatctctggctgaaagatgacggcggcgcggatatcaaaggttccgtggacgttcaggggcgcgaaggtagcatcgaagtggtggcgctggatcacgatgtgtacatcccgaccgacaataacaccggcaaactgaccggtacccgtactcacaagccttttacgtttaccaaagaaatcgatgcgtccagcccgtatctctacaaagctgtgaccaccggacagaccctgaaaacggcagaatttaagttttaccgcatcaacgatgccggtcaggaagtggagtacttcaacatcacgcttgataacgtcaagctggtcagagtcgctccgcttatgcacgacatcaaggatccttccagagagaagcataaccacctggaacgtattgagttccgctacgagaaaatcacctggacttacaaagacggcaacatcattcattccgactcgtggaatgagcgtccttccgcc

SEQ ID NO: 147 - DNA, 1650 bp, Escherichia coli
gtgaggaacacgctgaaacaggccatcgtgctgtggggaatggtgttactgctggtgctgtggtcagtgtttatcagtccgtctggcgtgctgagatgggccggtgcggcggctatcgttctggcggttgccgcgttgttgatttatcggcgcaggcaggcgtggacggagatgaccggcgatgccgggttgtcatcgctgccgccggaaacctaccgacagccggtagtgctggtctgtggcggtctgtcggcgcacctgtccactgacagcccggtccgccaggtttcagaagggctgtatctgcatgttcctgatgaagaacagcttgtggcgcaggtggagcgattgctgacccttcgcccggcgtgggcatcgcagcttgccgtggcgtataccatcatgcccggcatacaccgggatgtggcggttctggccggacggctgcgacggttcgcccacagtatggcgacggtgcgtcgtcgggcaggcgtaaacgtcccctggcttctctggagcgggctgtccggctcgccgttgccggaaagagcgagttcaccgtggtttatctgtaccggcggcgaagttcaggtagcaacatccacagagaccaccatgcccgcgcagtggattgcacaatccggcgtacaggagcgcagtcagcgactctgttacctgctgaaagctgaaagcctgatgcagtggctgaatcttaatgtgctgacggcactgaacggcccggaggcgaaatgtccaccactggcgatgaccgtggggctggtcccctcgttgcctgcggtggataacaacctgtggcagttgtggatcaccgccagaaccggcctgacgccggatatcgcggacaccggcacagacgatgcgctgccattcccggatgccctgttacggcagttgccgcgtcagtcgggctttaccccgctgcgacgagcctgcgtgaccatgctgggcgtcaccaccgtggcgggtatcgccgcgctgtgcctgtcagccacggcaaatcgccagttattacggcaggtcggtgacgatctgcaccggttttatgccgtcccggtggaggaatttatcaccaaagcccgtcacctgtcggtgctgaaagacgatgcgaccatgctcgatgggtattaccgggaaggagaacccctgcgcctcggtctggggttataccccggcgaacgcatccgccagccggtattacgcgccattcgcgactggcgtccgcctgaacaaaaaatggaggtgacggcttcgcttcaggttcagaccgtgcgtcttgacagtatgtcgctgtttgacgtcggacaggcccgcctgaaagacggctcgacaaaagtgctggtggacgcactggtgaacatccgggcaaaaccgggctggctgatcctcgtggccggatataccgatgccaccggcgatgaaaaaagcaatcagcagttatcgctgcggcgtgccgaagcggtgcgcaactggatgctgcagaccagcgacatcccggccacctgttttgccgtacagggactgggcgagagccagcctgcggcgaccaacgacacgccacagggccgggcagtcaaccggcgtgtcgaaatcagtcttgttccgcgttctgacgcctgtcaggacgtgaaa

SEQ ID NO: 148 - DNA, 582 bp, Escherichia coli
atgatcaaatccacattctggcgagcgctcgccctgaccgctacgcttatcctcactggctgtagccactcgcaaccggaacaggaaggccgcccgcaggcgtggctgcaacctggtacgctcatcacgctgcctgcgccggggatttcacccgcagtcaattcccagcaactgttgaccggcagcttcaacggcaaaacccagtctctgctagtgatgcttaatgccgaagatcagaaaatcacccttgccgggctgtcgtcggtcggcattcgcctgtttctggtgacctacgatgcaaaagggctacgcgccgagcaatccatcgtcgtcccacagttaccgcccgcaagtcaggtactggctgacgtgatgctcagccactggccgattagcgcctggcaaccgcaacttcccacaggctggacgcttcgcgacaacggcgacaaacgcgagctgcgtaacgccagcggcaaactggtcacggaaatcacctatctgaatcgccagggaaaacgcgtgccaatcagcattgagcagcatgtctttaaataccacatcaccattcaatacttaggtgac

SEQ ID NO: 149 - DNA, 387 bp, Escherichia coli
atgaaacgttatataaaatggtttgccatcacaatttttatcagtatgttgagtgcctgtgtccgtacggccccagtgcaacagataagcaccactgtcagtgtgggtcatactcaggagcaggttaaaaatgccattttgaaagcaggtgcgcagcgcaagtggattatgacgcaagtgtcccctggagttattaaagctcgctatcaaacacgaaatcacgttgcagaggttcgtattacatatacagctacctactataacatcaaatatgacagtagcctgaatctgcaggcttctgatggaaaaattcataaaaactataaccgctgggtgcgtaacctggataaagatatacaggttaacttatctacaggagcaacgtta

SEQ ID NO: 150 - DNA, 1245 bp, Escherichia coli
atgaagcgtaaacatttgttattattattgttgttttcattttccactaacagtgcgcctctttactccttaattagggaggcagttatgcacgatcccatagtaatggaagcccgggcggagttaacttcggcacaatcccgcatagagcaggcaagctctgcacattggccagttgtcacagctacaggaagtaaactcctttcacaaagtcaccgttattcctacgattatgacactgaagatattttacccggtattcgtggtgaagtgaatatatttgcttcaggggctattgaggcggatgtgcgtcggagtgagtcagaagccgaatattatcattataaaatggaagaaacaaaagaggaaacaattcactcttttgtttcattatatcttgatgcactcagggaaaaacaatccattgcggtacttgaacagagcctttcccggcataacgcaattcttaatgacctgaataccatcagtattcatgataccgggcgggagtctgagcttgttcaggccgaagccagaaggttgatggttcggcagcagataaattctaggagcagagtacttaaaaccacgctgggaaaactgtccacttggacaaaaaatccggtaaccgaagctgatcttgaaaatcctttttctaggatgacagaggccaaattattaactgattttacacaggctccacagaaaggtaacccgtcgtggcttgccagccaagctgatgttgagagtaaaaaagcggcactgaaagcacaggagcttgcccggtaccctcgggtggatttaacggggtctgtaacccgggatgaccagcagataggggtcaatctgtcttgggacctctttaaccgtaatgccagttatggtgttacagaaaaagctgcgcaaatagtggcagctaccggacgactggactctgtcgcccgaatgattgatgaaaccgggcgattatctctgataacagtcagacaaagtcgaggggaaatggaaacgctcagacgtcaggaacaggcttcagccagagttgtggacttttatcgtcttcagtttcaggtggcaagaaaaacactgattgaattactgaatgctgaaaacgaactgtacagtgtcggactctcccgggttcagacggaggatcagatgctccacggtatgctggattatctgtattcccagggaatgctcctgaaatggagcggagtgaatctttctggtgaagaagaaaaa

SEQ ID NO: 151 - DNA, 603 bp, Escherichia coli
atgaaatttttaccgctgctggcgctgctgattagcccgtttgtgagcgccctgaccctggacgatcttcagcaacgctttaccgaacaaccggtgatccgcgcccattttgatcaaacccggacgattaaagatctgccgcagccgctgcgatctcagggtcagatgttgatcgcccgcgaccaggggttattgtgggatcaaacctcaccgttccccatgcagctattgctggatgataaacgcatggtgcaggtgatcaacggtcagccgccgcaaatcatcacggcagaaaacaacccgcagatgttccagtttaaccacctgctgcgcgcgctgttccaggccgatcgcaaagtgctggaacaaaacttccgcgtcgaatttgctgacaaaggcgaaggccgctggacgctgcgcctgacgccgaccaccacgccgctggataaaattttcaacaccatcgatctcgccgggaaaacctatctggagagcattcaacttaatgataaacagggcgatcgcaccgatattgctcttacccaacatcaactgacgccagcgcaactgaccgatgacgaacaccaacgttttgccgcccag

SEQ ID NO: 152 - DNA, 2295 bp, Escherichia coli
atgaaaaacagtaaggtattttaccgcagcgcattagcgacagctattgttatggctctttctgcaccagcattcgctactgatagcacggtatcaactgatccggttacgctgaatacagagaagacgactctggatcaagatgttgttattaacggtgataacaagattacagccgtaacaattgaaacgtcagattcagataaagaccttaatgttacttttggcggtcacgatattaccgccgcatcaacggtaaaccaagatttcgttgaaggtgtaaaagttagtggtaacaaaaatgttgtgattaatgctacagactccaccatcacagctcaaggtgaaggcacctatgtccggactgcaatggtcattgattcaactggcgatgttgttgttaatggcggtaatttcgttgcaaaaaatgaaaaaggtagtgcgacagggatatctctggaagcgaccacgggaaataatttaacgctcaatggtacaaccataaatgctcaaggtaataagagttacagcaacggctctacggcaatttttgctcaaaagggtaatttgttgcagggttttgacggtgatgcaaccgacaacatcacccttgctgactcaaatattattaatggcgggattgaaacaatagttactgccgggaataagacgggaattcatacagtcaacctgaatattaaggatggctcagtaattggggcggctaataataaacaaacaatttatgcctctgcttcggcacaaggcgcaggttcagcaacgcaaaatttaaatttgtctgttgctgattcaaccatctactctgatgtcctggccctttctgaaagcgagaattcagccagtaccacaacaaatgtaaatatgaacgttgcccgctcttactgggaaggtaatgcttataccttcaatagcggcgataaagcgggtagtgatctggatataaatctttccgatagttcagtctggaaaggcaaagtttcaggggcaggagatgccagtgtatctctgcaaaacgggtctgtctggaatgttacgggttcctcaactgttgatgctctggcagtaaaagacagtacggttaatatcacgaaggctacagtcaatactggcacgtttgcttctcaaaacggcactctgattgttgatgcctcttctgaaaacactctggatatcagcggtaaagcgagcggtgacttgcgtgtttacagtgcgggttcattggatcttatcaatgaacaaacggcatttatttctaccggcaaagacagcactctaaaagccacaggcacaacggaaggtggtctgtatcaatatgacctgacacagggagctgatggtaacttttatttcgtaaaaaacacgcataaagcatccaacgccagctccgtgattcaggcaatggcagctgctccggctaacgtcgctaatctgcaggctgacacgctctccgcccgtcaggatgctgtccgtctgagcgaaaatgacaagggtggcgtatggattcagtactttggcggtaaacagaaacataccaccgcgggaaatgcatcctatgacctggatgtaaatggtgtaatgctgggtggtgatacccgcttcatgactgaagatggtagctggctggccggtgtggcgatgtcttctgcgaaaggtgacatgactaccatgcagagcaaaggtgacactgaaggttacagcttccacgcttacctgagccgccagtataacaacggtatcttcattgatactgctgcacagtttggtcactacagcaacacggcagatgttcgcctgatgaatggtggcggtaccatcaaagctgactttaacaccaatggttttggtgcgatggttaaaggcggttacacatggaaagacggtaatggcctgtttattcagccatatgccaaactgtctgctctgactctggaaggtgtggattatcaactcaacggcgtggacgttcattctgacagctataactctgtgctgggtgaggccggtacgcgcgtgggttatgacttcgctgtgggcaacgcgaccgttaaaccttatctgaatctggccgcactgaacgaattctctgatggcaacaaagtccgtctgggtgatgagtctgtcaatgccagcattgacggtgcagcattccgcgtgggtgcaggtgtacaagctgatatcaccaaaaacatgggagcatatgcaagccttgactacaccaaaggtgacgacattgagaacccgctacagggtgtagttggtatcaatgtgacctgg

SEQ ID NO: 153 - DNA, 1980 bp, Escherichia coli
atgtcacgtccgcaatttacctcgttgcgtttgagtttgttggctttggctgtttctgccaccttgccaacgtttgcttttgctactgaaaccatgaccgttacggcaacggggaatgcacgtagttccttcgaagcgcctatgatggtcagcgttatcgacacttccgctcctgaaaatcaaactgctacttcagccactgatttgctgcgtcatgttcctggaattactcttgatggtaccggacgaaccaacggtcaggatgtaaatatgcgtggctatgatcatcgcggcgtgctggttcttgtcgatggtgttcgccagggaacggataccggacacctgaatggcacttttctcgatccggcgctgatcaagcgtgttgagattgttcgcggaccttcagcattactgtatggcagtggcgcgctgggtggagtgatctcctacgatacggtcgatgcaaaagatttattgcaggaaggacaaagcagtggttttcgtgtctttggtactggcggcacgggggaccatagcctgggattaggcgcgagcgcgtttgggcgaactgaaaatctggatggtattgtggcctggtccagtcgcgatcggggtgatttacgccagagcaatggtgaaaccgcgccgaatgacgagtccattaataacatgctggcgaaagggacctggcaaattgattcagcccagtctctgagcggtttagtgcgttactacaacaacgacgcgcgtgaaccaaaaaatccgcagaccgttgaagcttctgatagcagcaacccgatggtcgatcgttcaacaattcaacgcgatgcgcagctttcttataaactcgccccgcagggtaacgactggttaaatgcagatgcaaaaatttactggtcggaagtccgtattaatgcgcaaaacacggggagttcaggcgagtatcgtgaacagataacaaaaggagcaaggctggagaaccgttccactctatttgccgacagtttcgcttctcacttactgacatatggcggtgagtattatcgtcaggaacaacatccgggtggcgcgacgacgggcttcccgcaagcaaaaatcgattttagctctggttggctacaagatgagatcaccttacgcgatctgccgattaccctgcttggcggaacccgctatgacagttatcgcggtagcagcgacggctacaaagatgttgatgccgacaaatggtcatctcgtgcggggatgactatcaacccgaccaactggctgatgttatttggctcatatgctcaggcattccgcgccccgacgatgggcgaaatgtataacgattctaaacacttctcgattggtcgcttctataccaactattgggtgccaaacccgaacttacgtccggaaactaacgaaactcaggagtacggttttgggctgcgttttgatgacctgatgttgtccaatgatgctctggaatttaaagccagctactttgataccaaagcgaaagattatatctccacgaccgtcgatttcgcggcggcgacaactatgtcgtataacgtcccgaacgccaaaatctggggctgggatgtgatgacgaaatataccactgatctgtttagccttgatgtggcctataaccgtacccgcggcaaagacaccgataccggggaatatatctccagcattaacccggataccgttaccagtaccctgaatattccgatcgctcacagcggcttctctgttggttgggtcggtacgtttgccgatcgctcaacacatatcagcagcagctacagcaaacaacctggctatggtgtgaatgatttctacgtcagttatcaagggcagcaggcgctcaaaggcatgaccactactctggtattgggcaacgccttcgataaagagtactggtcgccgcaaggcatcccacaggatggtcgtaacggaaaaattttcgtgagttatcaatgg

SEQ ID NO: 154 - DNA, 2157 bp, Escherichia coli
atgagggatgaaatgttatataatataccttgtcgaatttatatcctttccactctgtcattatgcatttctgggatagtttctactgcaaccgcaacttcttcagaaacaaaaatcagcaacgaagagacgctcgtcgtgaccacgaatcgttcggcaagcaacctttgggaaagcccggcgactatacaggttattgaccaacaaacattgcagaactccaccaatgcctccatagccgataatttgcaggacatccccggagtagagataacagacaactccttggcaggccgtaaacaaatccgcattcgtggcgaagcatcctcccgtgttttaattctcattgatggtcaggaggtaacttatcagcgcgccggagataattatggtgtgggactgttgatagatgagtctgcgctggagcgtgttgaggtagtgaaaggtccatattccgtactgtacggttcacaggcaattggcggtattgttaacttcataaccaaaaagggaggtgacaaacttgcatctggagttgtgaaagctgtttataattccgcaacagcaggctgggaagaatcaatcgcggtccaggggagcatcggtggatttgattatcgcatcaacggtagttattctgatcagggcaatcgtgatacgccggatggacgtctgccgaataccaactatcgtaacaatagtcagggtgtatggttgggttataactccggaaaccatcgttttggcctctcgcttgatcgctacagactcgcgacgcaaacttactatgaggatccagacggaagctatgaggcatttagtgtcaaaatacctaaacttgaacgagagaaagttggggtattctatgacacagacgtggacggtgactatctaaaaaaaattcatttcgacgcgtatgagcagaccatccagcgccaatttgccaacgaagtaaaaacgacacagcctgttcccagtccgatgattcaggctctgaccgttcataacaagactgacacccatgataagcaatacactcaggcggtcacattgcagagtcacttttcgctgcctgctaataatgaacttgttaccggtgcacagtacaaacaagacagggtcagccaaaggtccggtggcatgacctcaagcaaatctctgaccggcttcattaataaggaaacacgaactcgctcctattatgagtcagagcaaagtacagtctcactattcgcacaaaatgactggcgattcgccgatcactggacatggacaatgggagttcgccaatactggctttcttcaaagttgacgcgtggtgacggagtatcatataccgcaggcattataagcgatacctctcttgccagagagtctgcgagtgatcacgaaatggtaacatctacaagcctgcgctattcaggtttcgataacttggagttacgcgctgcgttcgcgcaaggctacgtatttcccacactctcccagctttttatgcagacatctgcgggcggcagtgtcacatacggaaatcctgatcttaaggctgaacactccaataactttgaattaggtgcacgatataatggtaatacgtggctgattgacagcgcagtttactactcagaagctaaagattatattgcaagtctgatctgtgatggcagtatagtttgcaatggtaacaccaactcctcccgtagtagctactattattatgacaatattgatcgggcaaaaacatggggactggaaataagcgcggaatataatggctgggttttctcgccatatatcagtggcaatttaattcgtcggcaatatgaaacttcaacattaaaaacaactaatacaggagaaccagcgataaacggacgtatagggctgaaacatactcttgtgatgggtcaggccaacataatctctgatgtttttattcgtgctgcctctagtgcaaaagatgacagtaacggtaccgaaacaaatgttccgggctgggccactctcaactttgcagtaaatacagaattcggtaacgaggatcagtcccggattaacctagcactcaataacctgacagacaaacgctaccgtacagcacatgaaactattcctgcagcaggttttaatgcagctataggttttgtatggaatttc

SEQ ID NO: 155 - DNA, 600 bp, Escherichia coli
atgcgtaaagtttgtgcagtcattttgtccgcagccatctgtctgtccgtatccggtgcgcctgcatgggcgtctgaacatcagtccacactgagcgcggggtatcttcatgcccgtacgaacgctcccggcagcgataatctgaacgggattaacgtgaaataccgttatgagtttacggacgcgctggggctgattacgtccttcagttatgccaatgctgaggatgagcaaaaaacgcactacagcgatacccgctggcatgaagattccgtgcgtaaccgctggttcagcgtgatggcggggccgtctgtacgcgtgaatgaatggttcagcgcgtattcgatggcgggtgtggcttacagccgtgtgtcgactttctccggggattatctccgcgtaactgacaacaaggggaaaacgcacgatgtgctgaccggaagtgatgacggtcgccacagcaacacgtctctggcgtggggggctggcgtgcagtttaacccgaccgaatccgtgaccattgaccttgcttatgaaggttccggtagtggcgactggcgaacggatgcatttattgttggtatcggataccgtttctga

SEQ ID NO: 156 - DNA, 1368 bp, Escherichia coli
atgaaaaaatcgacattatctttagccatcggtttattattggcatgtagtaccggtatggcaaaaacacagcatttaacgctggaacaacgcctggaagcggcagaaatgcgggcagcaaaagcagaggggcaggttaaacagcttcagacacaacaagccgccgagatccgcgaaattaaaaccgcacagggcaacacgccggtaaacggtcaatcaacgacggagtcagagaagaaaaacgccaccccgcctaatctcctgctttcagggtatggcgatttaaaaatctacggtgacgtagaatttaatatggatgcggaaagtaatcatggcctgctggcaatgaccaacgctgatgtgaatagcgatcccactaatgaatggaatctcaatggtcgtattctgttaggttttgatggtatgcgaaaactggataatggctatttcgctgggttctccgcacaaccgctgggggatatgcacggttcagtaaatatcgatgatgcggttttcttctttggcaaagaaaacgactggaaggtcaaagtaggccgttttgaagcctacgatatgttcccgctgaatcaggatacctttgttgaacattccggtaatactgcgaacgatctttatgacgatggcagcggttatatctatatgatgaaagagggccgcggacgttctaacgctggcggtaatttcctcgtcagcaaacaactcgataactggtattttgaattaaacacgttactggaagacggaacatctttatataacgacggtaattatcatggacgcgatatggaacagcagaaaaatgttgcttatctgcgtccggtaattgcctggtcgccgacggaagaattcaccgtttccgcagcgatggaagcgaatgtggtaaataatgcttatggttataccgatagcaagggtaattttgtcgatcagtccgatcgtaccggttatggcatgagtatgacctggaatggcctgaaaaccgatccggaaaatggcatcgtggttaatcttaataccgcctatttagatgctaataatgaaaaagatttcacggcagggattaacgcgctgtggaaacgtttcgagctgggttatatctatgcacataataagattgatgaatttagtggcgtggtttgtgataacgattgctggattgatgatgaaggaacatacaacattcacaccattcatgcgtcttatcagttcgctaatgtgatggatatggagaactttaatatttacctcggcacgtattactccattctggatagcgacggcgataagatacacggcgacgatagtgatgaccgttacggcgcacgcgttcgctttaaatacttcttc

SEQ ID NO: 157 - DNA, 522 bp, Escherichia coli
atgaacggcaaagcgtttctggcctgcgttctgatgagcgtcgtattaactggctgtgaaacagcgaaaaaaatcagccaggtgatccgcaatccggatattcaggtcggaaagctgatggatcagtcaaccgagctgaccgtcacgctgctgaccgagccggacagcaacctgacggcggatggcgaagccgcgccggtggatgtccagttggtttatctgagcgacgactcaaaattccatgccgccgactacgaccaggttgccaccaccgcgctgcccgacgtgctggggaaaaactatatcgatcaccaggacttcaacctgttgccggataccgtaaaaacactgccgccgatcaagttggatgagaaaaccggttatatcggtgtcattgcctatttttcagacgaccaggccacagaatggaaacaaattgagtcggtagaaagtatcggccaccactatcgcctgctggtgcatatccgcgccagtgcgattgagatgaaaaaagaggaaaac

SEQ ID NO: 158 - DNA, 3432 bp, Escherichia coli
ctgacgctggcatggatttttctgctggtgtggatctggtggcagggtccaaaatggacgctctatgagcagcactggctggctccgctggcaaaccgctggctggcgaccgccgtctggggacttatcgctctggtctggctcacctggcgggtgatgaagcgtctgcaaaagctggaaaaacagcagaaacagcagcgggaggaagaaaaagatccgttgaccgtggaactccaccgccagcagcaatatctggatcactggctgctgcgcctgcgccgccatctggataaccgccgttatctgtggcagttgccgtggtatatggtcattggtcctgcgggtagcggcaaaagcacgctgctgcgcgagggctttccgtctgacattgtttacacgccggaaagcatccggggtgtggaataccacccgctgatcacaccgcgagtgggcaaccaggcggtaattttcgatgttgacggcgtactgaccactcccggcggggatgatctgctccgccgccgcctgcgcgaacactggctgggctggctgatgcaaacgcgcgctcgccagccgctcaacggtcttatcctgacgctcgatcttcccgatctgctgacggcggataaatcccgccgtgagacactggtacaaaatttgcgccagcaacttcaggagatccgtcagagcctgcactgccgtctgcccgtttacgtggtgctgacacggctggatctgctgaacggctttgccgcgctgttccattcactggataaaaaagaccgcgatgcgatcctcggcgtcacatttacccgccgcgcccatgaaagtgacggctggcgcagcgaactgggggctttctggcagacgtgggtacaacaggtgaacctggcgctgtcggatctggtgctcgcacaaaccggtgctgctccccgcagcgctgtgttcagcttctcccgtcagatgcagggaacaggagaaatcgtcaccgcactgctcgccgcattgctggacggtgagaacatggatgtaatgctgcgtggcgtctggctcacatcctcgctacagcgtggccaggtggatgatattttcacgcagtccgccgcccgccagtacggactgggtaacagctcgctggcaacctggcctctggtggagacgacgccgtattttactcgccgcctcttcccggaagtcctgctggctgagccgaacctggcgggtgaaaacagcgtctggctgaacagctcccggcgcaggctgaccgccttttccacctgtggcgcggcactggcggcattgatggtcggaagctggcaccattattacaatcagaactggcagtctggcgttaacgtactggcacaagctaaagcctttatggacgtaccaccaccgcagggaacggatgaattcggcaatctgcaattgccattgcttaacccggtacgcgatgccaccctggcctatggtgattatcgcgatcacggttttctggcggatatgggattgtaccagggcgcccgcgtagggccgtatgtggagcaaacctacattcagcttcttgagcagcgttatctcccctcgttaatgaacggcctgatccgggatctaaacattgccccgccagagagcgaagaaaagctcgctgtgctgcgcgtagtgcgcatgatggaagacaaaagtgggcgcaacaacgaggcggtaaaacagtacatggcacggcgctggagcaatgaatttcacggccagcgcgatattcaggcgcaactgatggtgcatctggactatgcgctggagcacaccgactggcacgcgcagcgccaaagcagcgacagcgatgctgtcagccgctggaccccctatgataaaccgatcattaatgcgcagcaggaactgagcaagctgcccatataccagcgtgtctaccagaccctgcgcaccaaagcattaagcgtgttgcccgccgatttgaatttgcgcgaccaggttggtcccaccttcgacaacgtgttcgtcgccggtaatgatgaaaaactggtgatcccgcagttcctcacccgctatggactgcaaagctattttgtcaaacagcgtgagggcctcgttgagctgaccgcgctggattcgtgggtactgaacctgacgcaaagcgtcgcctacagcgaggccgaccgtgaagagatccagcgccatatcaccgaacagtacatcagtgactataccgccacctggcgtgccggaatggataacctcaacgtccgtgactatgaggccatgtcggcgctgaccgacgcgctggagcagattatcagcggcgatcagccattccagcgtgcgctgacggcgctgcgcgataatacccacgcgctgacgctctccggcaaactggatgataaggcgagggaagcggcgataaatgagatggattaccgcctgttatcccggctggggcatgagttcgcaccggaaaacagcgcactggaggagcaaaaggacaaggcgagtacgctacaggccgtgtaccagcaactgaccgagctgcaccgttacctgctggcgatccagaactcgccagtgccggggaaatcggcgctgaaagcagtacagctacggctggatcaaaacagcagcgatccaatcttcgccacccgtcagatggcaaaaaccctgcctgcgcctcttaaccgctgggtaggtaagctcgcggatcaggcctggcatgtggtgatggtggaagccgttcgttacatggaagtggactggcgcgacaatgtagtgaaacccttcaacgagcagcttgccgataactatccgtttaatccgcgcgccacacaggatgcctcactggattcgtttgaacgtttctttaaaccggatggcattctggacaatttctacaagaacaacctgcgcctgttccttgaaaacgatctgacctttggcgacgacggcagagtgttaatccgtgaagatatccggcagcaactggataccgcgcagaaaatccgcgacatcttcttcagccagcagaacgggctgggcgcacagtttgccgtggaaaccgtatcgctttccggcaataagcggcgcagcgtacttaacctggacggccagttagtggactacagccagggacgcaactacaccgcccatctggtctggccgaacaacatgcgtgaaggcaatgaaagcaagctgacgctgattggcaccagcggcagagcaccgcgcagtatcgcgttcagtggaccgtgggcgcagttccgcctgttcggcgcgggccagttgaccaatgtgaccagtgacacctttaacgtgcgctttaacgtggacggcggcgcaatggtttaccaggtgcatgtggataccgaagataacccgttcaccggcggtctgttcagcctgttccgtttaccggatacgttgtat

(19) United States
(12) Patent Application Publication
(10) Pub. No.: US 2010/0260789 A1
(43) Pub. Date: Oct. 14, 2010

(54) PRODUCTS SPECIFIC TO PATHOGENIC STRAINS AND THEIR USE AS VACCINES AND IN IMMUNOTHERAPY

(75) Inventor: SONIA ESCAICH, PARIS (FR)

Correspondence Address: NIXON & VANDERHYE, PC, 901 NORTH GLEBE ROAD, 11TH FLOOR, ARLINGTON, VA, 22203, US

(73) Assignee: MUTABILIS, PARIS (FR)

(21) Appl. No.: 12/481,786

(22) Filed: Jun. 10, 2009

Related U.S. Application Data
(63) Continuation of application No. 10/506,666, filed on Oct. 27, 2004, now abandoned.

(30) Foreign Application Priority Data
Mar. 6, 2002 (EP) 02290556.6

(51) Int. Cl. A61K 39/108; C07K 14/00; C12Q 1/04; A61P 37/04; A61P 31/04

(52) U.S. Cl. 424/190.1; 530/350; 435/34

(57) ABSTRACT

The invention relates to isolated antigenic polypeptides obtainable by a process comprising the steps of: 1—selecting on the basis of sequence analysis those of the polypeptides which are either located in the outermembrane or secreted by the bacteria, 2—identifying the genes coding for said polypeptides which are conserved in B2/D clinical isolates, 3—purifying the polypeptides identified in step 1, which are found in step 2 to be conserved in B2/D isolates, 4—testing the polypeptides for immunogenicity using animals models. Application for making vaccines compositions and immunotherapies.

This application is a continuation of application Ser. No. 10/506,666 (U.S. Patent Application Publication No. 2006/0188514 A1), filed Oct. 27, 2004, which is a U.S. national phase of International Application No. PCT/EP03/02925 filed 6 Mar. 2003, which designated the U.S. and claims priority to European Application No. 02290556.6 filed 6 Mar. 2002, the entire contents of each of which is hereby incorporated by reference in this application.

The invention relates to new products specific to pathogenic strains, particularly to extra-intestinal E. coli strains.

It more particularly relates as products to antigenic polypeptides and antibodies directed against said polypeptides and to their use as vaccines and in immunotherapy, respectively.

Although Escherichia coli is probably the best known bacterial species and is one of the most common isolated in clinical microbiology laboratories, misconceptions abound regarding the various types of E. coli and the infections they cause.

E. coli strains of biological significance to humans can be broadly classified in 3 major groups:
1. Commensal strains, which are part of the normal flora.
2. Intestinal pathogenic strains, which are not part of the normal flora. This group contains various pathotypes (EPEC, EHEC, ETEC, EIEC) not including Shigella.
3. Extra-intestinal strains (ExPEC) which are responsible for infections outside the gastro-intestinal (GI) tract, but can also be part of the normal flora. All hosts, either immunocompromised or not are susceptible to these infections.

ExPEC strains are responsible for the majority of the urinary tract infections (UTI) particularly cystitis, pyelonephritis, and cathether associated infections.

They are also responsible for abdominal infections, nosocomial pneumoniae, neonatal meningitidis, soft tissue infections, and bone infections. Each one of these localizations can lead to bacteremia with a risk of sepsis in case of organ failure. ExPEC strains are indeed the most common Gram negative bacilli isolated from blood cultures.

750 000 cases of bacterial sepsis occur each year in the US, and are responsible for 225 000 deaths. In a recent study on 1690 cases of sepsis, it was shown that the main bacteria species identified is ExPEC (16% of the cases) and then S. aureus (14% of the cases).

These numbers demonstrate the importance of ExPEC strains in both hospital and community acquired infections.

ExPEC strains correspond to a homogenous subset of E. coli strains. Analysis of phylogenetic relationships among E. coli strains by MLEE has revealed that E. coli belong to 4 main phylogenetic groups designated A, B1, B2 and D.

The pathogenesis of ExPEC strains is that of extra-cellular microorganisms, i.e., they are well adapted to growth in the extra-cellular fluids and efficiently resist phagocytosis by polymorphonuclear. Initial studies have shown that virulence factors known to be important for the extra-cellular growth are mainly found in B2/D E. coli., thus suggesting that B2/D subgroups contain most of the ExPEC strains. This was reinforced by experiments performed on animals showing that B2/D strains are more virulent than A and B1 strains.

Subsequent epidemiological studies have indeed confirmed these hypotheses. B2/D isolates are those predominantly responsible for neonatal meningitidis (87%) and community or nosocomial acquired urosepsis, (93% and 85%, respectively).

Similar results have been reported for cystitis (70% are due to the sole B2 E. coli), thus demonstrating that the importance of ExPEC strains.

These recent findings demonstrate that the B2/D subgroup of strains is the E. coli core genome the best adapted to growth in extra-cellular fluids.

In addition to this core genome, ExPEC strains have various pathogenicity islands which encode virulence factors associated with the different pathogenesis of extra-intestinal E. coli infections (UTI, urosepsis, neonatal meningitidis . . . ). Among the main virulence factors are the capsule, which is well-known to be important for extra-cellular growth, and the iron chelation systems (aerobactin and enterochelin, for example). In addition, depending on the pathogenesis, these strains can produce toxins (CNF, hemolysin . . . ), adhesins (pap, sfa . . . ) and other iron chelation systems.

The notion that B2/D E. coli correspond to a distinct subset of pathogenic E. coli strains is reinforced by the fact that B2/D E. coli are not broadly isolated from the stools of humans. They were recovered from only 11% of individuals, whereas A and B1 subgroups are present in the stools of 74% of the individuals of a human population.

As mentioned above the pathogenesis of ExPEC strains relies on their ability to multiply in the extra-cellular fluids and to resist bactericidal activity of the complement and phagocytosis by polymorphonuclear. Therefore, as for other extra-cellular pathogens (Haemophilus influenzae, Streptococcus pneumonieae and Neisseria meningitidis) a protective antigen against ExPEC has to induce antibodies that promote opsonisation and/or the bactericidal activity of serum.

Considering the above statements, an efficient antigen has to be largely represented among the population of 132/D E. coli. Similarly to other extra-cellular pathogens, the capsular polysaccharide would be an ideal antigen, however most pathogenic B2 strains express the K1 polysaccharide. The latter has a structure identical to that of group B meningococcus, which is non-immunogenic and shares common antigens with the brain. Another possible target may be the lipopolysaccharide (LPS). However there are a large number of different LPS serotypes that are shared by various subgroups.

The inventors have now found that some specific components coded by the B2/D genome, but absent from A and B1 E. coli strains, are particularly useful as antigens and can specifically prevent the pathologies due to ExPEC strains. Homologs of these antigenic components can be found in other pathogenic bacterial species and therefore are useful to prevent the pathologies caused by these bacteria. Accordingly, any reference to products specific to ExPEC strains and to their uses will encompass components in these species.

For example homologuous antigens could be present in the following species and be as such used for prevention of disease due to the bacteria:

Pseudomonas aeruginosa, Escherichia coli O157:H7, Yersinia pestis, Vibrio cholerae, Legionella pneumophila, Salmonella enterica, Salmonella typhimurium, Haemophilus influenzae, Neisseria meningitidis, Neisseria gonorrhoeae, Bacillus anthracis, Burkholderia cepacia, Campylobacter jejuni, Chlamydia pneumoniae, Chlamydia trachomatis, Clostridium botulinum, Clostridium difficile, Cryptococcus neoformans, Enterobacter cloacae, Enterococcus faecalis, Helicobacter pylori, Klebsiella pneumoniae, Mycobacterium leprae, Mycobacterium tuberculosis, Pseudomonas aeruginosa, Salmonella paratyphi, Salmonella typhi, Staphylococcus aureus, Klebsiella pneumoniae, Listeria monocytogenes, Moxarella catarrhalis, Shigella dysenteriae, Shigella flexneri, Shigella sonnei, Staphylococcus epidermidis, Streptococcus pneumoniae, and any species falling within the genera of any of the above species.

It is then an object of the invention to provide new isolated antigenic polypeptides, and polynucleotides belonging to the core B2/D genome and not present in commensal E. coli.

Another object of the invention is to provide antibodies raised against such antigenic polypeptides, or peptidic fragments.

It is still another object of the invention to provide vectors and host cells containing said polynucleotides.

Another object of the invention is to provide vaccine compositions specific to extra intestinal infections caused by ExPEC and pathologies caused by other pathogenic strains expressing antigenic polypeptides homologous to the ExPEC antigenic polypeptides.

The invention also relates to means for detecting and treating a development of E. coli in a human or animal compartment which is extra-intestinal (systemic and non-diarrhoeal infections, such as septicaemia, pyelonephritis, or meningitis in the newborn).

The isolated antigenic polypeptides used according to the invention are selected among polypeptides specific to B2/D E. coli strains and not present in A and B1 isolates of E. coli. They are encoded by genes belonging to the core B2/D genome and are not present in commensal E. coli.

They have a sequence selected in the group comprising the sequences of SEQ ID NO:11 to NO:66 or 133-145 or homologous sequences with a minimum of 25% of identity with the whole sequences SEQ ID NO:11 to NO:66, or 133-145, respectively.

The isolated polypeptides having SEQ ID NO: 14, 15, 17, 21, 22, 23, 28, 29, 30, 32, 36, 38, 39, 41-44, 46, 49, 50, 52 to 55, 58, 60, 63, 133-138 are new polypeptides and therefore are part of the invention.

The invention also relates to homologous isolated antigenic peptides, comprising polypeptides having at least 25% identity to a polypeptide having a sequence SEQ ID NO: as above defined, more particularly having SEQ ID NOs: 14, 15, 17, 21, 22, 23, 28, 29, 30, 32, 36, 38, 39, 41-44, 46, 49, 50, 52 to 55, 58, 60, 63, 133-138, or at least 25% identity to a fragment comprising at least 5, at least 10, at least 20, at least 30, at least 40, at least 50, at least 60 or more than 60 consecutive amino acids of a polypeptide having a sequence corresponding to said SEQ ID NOs:, as determined using BLASTP or BLASTX with the default parameters.

Said polypeptides are obtainable by a process comprising the steps of:
a—selecting on the basis of sequence analysis those of the polypeptides which are either located in the outermembrane or secreted by the bacteria,
b—identifying the genes coding for said polypeptides which are conserved in B2/D clinical isolates,
c—purifying the polypeptides identified in step a, which are found in step 2 to be conserved in the B2/D isolates,
d—testing the polypeptides for immunogenicity using animals models.

By the term “conserved”, it is meant, according to the invention, that the genes coding for the polypeptides are present with a frequency of at least 50% in B2/D isolates, preferably greater than 60%, more preferably greater than 80% and even more preferably greater than 85%, and in less than 40% in A/B isolates, preferably in less than 20%, more preferably in less than 15%.

The animal models used in step c are infected adult animals, eventually immunodepressed.

The adult animals particularly mice, are infected intraperitoneally, the endpoint being the animal death and/or bacteremia measurement.

The animals can be immunodepressed by injection, for example, of cyclophosphamide which induces a neutropenia. Such a model will validate the use of the antigen for prevention of E. coli sepsis in immunodepressed patients. Another animal model could be for example 2 to 3 day old infant mice.

The variants or fractional sequences conserving the B2/D properties and which are antigenic as defined in step 4 of the above process are also part of the invention. The term “variant” is herein intended to mean any sequence having insertions and/or deletions and/or substitutions with respect to the parent sequence. The term “fractional” is herein intended to mean any fragment of the parent sequence.

The invention also relates to the use of isolated polynucleotides coding for a polypeptide such as above defined according to the universal genetic code and taking into account the degeneracy of this code. The term “polynucleotide” encompasses any nucleotidic sequence such as DNA, including cDNA, RNA, including mRNA.

Said polynucleotides have preferably sequences corresponding to SEQ ID NO:77 to SEQ ID NO:132 or 146 to 158.

More preferably, said polynucleotides have sequences corresponding to SEQ ID NO: 80, 81, 83, 87, 88, 89, 94, 95, 96, 98, 102, 104, 105, 107-110, 112, 115, 116, 118, 119, 126, 127, 130, 132, 135, 146-151.

The invention also relates to the homologs to said polynucleotides. Said homologs may have at least 25% identity to a polynucleotide having said sequences, or at least 25% identity to a fragment comprising at least 15, at least 30, at least 60, at least 90, at least 120, at least 150, at least 180 or more than 180 consecutive nucleotide of a polynucleotide having one of said SEQ ID NOs:, as determined using BLASTN with the default parameters, and are encompassed by the invention inasmuch as they are capable of coding for a polypeptide having the antigenic properties of those according to the invention.

The present application is also aimed towards any vector comprising at least one of said polynucleotides and also any cell transformed by genetic engineering, characterized in that it comprises, by transfection, at least one of said polynucleotides and/or at least one vector according to the invention, and/or in that said transformation induces the production by this cell of at least one polypeptide corresponding to a polynucleotide such as above-defined.

The invention also relates to a process for isolating and identifying antigenic polypeptides, therefore useful as vaccine for E. coli.

Such a process comprises the steps of
a—selecting on the basis of sequence analysis those of the polypeptides which are either located in the outermembrane or secreted by the bacteria,
b—identifying the genes coding for said polypeptides which are conserved in B2/D clinical isolates,
c—purifying the polypeptides identified in step a, which are found in step 2 to be conserved in B2/D isolates,
d—testing the polypeptides for immunogenicity using animals models.

The selected antigenic polypeptides, alone or in combination, are capable of inducing an antibody response for prevention of infections due to ExPEC strains regardless of the pathogenesis and of the infection site (UTI, pyelonephritis, sepsis, bacteremia, neonatal meningitis).

Such polypeptides particularly have sequences SEQ ID NO:1 to SEQ ID NO:66, or 133-145 or correspond to homologous sequences.

The invention thus relates to vaccine compositions specific to E. coli extra-intestinal infections, comprising an effective amount of at least one antigenic polypeptide or fragment thereof as above defined, with a carrier, particularly at least one polypeptide of SEQ ID NO:1 to SEQ ID NO:66, except SEQ ID NO:8, and 133-145 and the homologous polypeptides.

Such vaccine compositions are particularly useful for preventing urinary system infections, pyelonephritis, sepsis, bacteremia, neonatal meningitis.

The vaccine compositions of the invention are indicated for:
immunodepressed patients, ideally before the start of the immunosuppressive therapy: patients suffering from cancer, diabetes, leukaemia, transplant patients, patients receiving long-term steroids therapy.
Patients before surgery where there is a high risk of E. coli infections (abdominal surgery).
In all these cases, the E. coli vaccine of the invention could be administered in association with a Staphylococcus aureus vaccine,
Patients with recurrent UTI, especially after one episode of pyelonephritis.
The prevention of neonatal infections will require vaccination of the mother, implying vaccination long before pregnancy to avoid potential problem. Ideally such a vaccine should be associated with a Group B Streptococcus polysaccharide vaccine in order to also prevent late onset neonatal infections. It should be pointed out that the induction of a level of antibodies against B2/D E. coli in pregnant women would also prevent UTI, which are always a risk in the context of a pregnancy.

The formulation and the dose of said vaccine compositions can be developed and adjusted by those skilled in the art as a function of the indication targeted, of the method of administration desired, and of the patient under consideration (age, weight).

These compositions comprise one or more physiologically inert vehicles, and in particular any excipient suitable for the formulation and/or for the method of administration desired.

For example the vaccine could be a suspension of the purified polypeptide in sterile water with aluminium based mineral salt as adjuvant and be administered subcutaneously with a first and boosting injection.

The antibodies raised against the above-identified polypeptides are also part of the invention.

They are capable of binding to said polypeptides in physiological-type conditions (in vivo or mimicking in vivo) when administered to a human or animal organism, and ELISA-type conditions when said binding product is intended to be used in assays and methods in vitro. Such antibodies advantageously inhibit the extra-intestinal growth of ExPEX strains in human or animal.

They are particularly useful for immunotherapy applications with antibodies specific to polypeptidic antigens, for treatment and prevention of severe infections in at risk populations such as neonates or patients undergoing surgical procedures. For these applications specific human monoclonal antibody (Mab) will be derived from the peptides or polypeptides.

The methods for manufacturing such antibodies using the polypeptides according to the invention are available to those skilled in the art. They are conventional methods which comprise, in particular, the immunization of animals such as rabbits and the harvesting of the serum produced, followed optionally by the purification of the serum obtained. A technique suitable for the production of monoclonal antibodies is that of Köhler and Milstein (Nature 1975, 256:495-497).

Said antibodies do not recognize the cells of the human or animal to which it is intended.

In particular for immunotherapy applications with monoclonal antibodies specific to polypeptidic antigens, for treatment and prevention of severe infections in at risk populations such as neonates or patients undergoing surgical procedures. For these applications specific human monoclonal antibody will be derived from the peptides or polypeptides.

The antibodies or fragments thereof are advantageously humanized when intended for a human administration.

Alternatively, humanized Mab could be derived from murine or rat Mab specific of the antigen. These fully humanized Mab are constructed using conventional molecular techniques to graft complementarity-determining regions from the parent murine or rat antibacterial antibody into human IgG1 kappa heavy and light-chain frameworks.

The present invention is also aimed towards the use, in an effective amount, of at least one of polypeptides having SEQ ID NOs: 14, 15, 17, 21, 22, 23, 28, 29, 30, 32, 36, 38, 39, 41-44, 46, 49, 50, 52 to 55, 58, 60, 63, 133-138, antibodies or polynucleotides for the diagnosis of the presence or absence of undesirable extra-intestinal E. coli, and/or for the diagnosis of an extra-intestinal E. coli infection.

The detection of the presence or absence of such compounds can in particular be carried out by nucleotide hybridization, by PCR amplification or by detection of their polypeptide products. Detection of the presence of such compounds makes it possible to conclude that a B2/D E. coli strain is present.

The invention also relates to pharmaceutical compositions for alleviating and/or preventing and/or treating an undesirable growth of E. coli comprising an effective amount of at least one polypeptide as above defined, particularly having SEQ ID NOs:1-66 to 133-145, in combination with a pharmaceutically acceptable carrier.

Preferred pharmaceutical compositions comprise at least one polypeptide having SEQ ID NOs:14, 15, 17, 21, 22, 23, 28, 29, 30, 32, 36, 38, 39, 41-44, 46, 49, 50, 52 to 55, 58, 60, 63, 133-138,

The present application is also aimed towards any use of a polypeptide such as above defined for the manufacture of a composition, in particular of a pharmaceutical composition, intended to alleviate and/or to prevent and/or to treat an undesirable growth of E. coli, such as an E. coli infection, (for example systemic and non-diarrhoeal infections), the presence of extra-intestinal E. coli or a sanitary contamination.

### BRIEF DESCRIPTION. OF THE DRAWINGS

The present invention is illustrated by the examples which follow and which are given in a non limiting capacity and with reference to FIGS. 1 and 2, wherein

FIG. 1 represents a protein purification result after cloning and expression, and

FIG. 2 is a picture of the DNA array after hybridization with the genomic DNA from a B2/D reference strain.

### EXAMPLE 1

### Assay for the Immunogenicity of a Selected Polypeptide from Sequences 1-66 and 133-145 (Except SEQ ID NO:8)

Cloning Expression and Purification of the Selected Polypeptide.

The nucleic acid having SEQ ID NO:95 encoding the polypeptide corresponding to SEQ ID NO:28 was cloned without the signal sequence (coding the 16 first amino acids) in a prokaryotic expression vector according to classical methods for cloning. The recombinant plasmid was used to transform the E. coli strain BL21. Transformed cells containing the recombinant plasmid were selected in LB medium with 100 μg/ml ampicillin. Individual clones are picked and grown in presence of IPTG 1 mM to induce recombinant protein expression. Total protein content of the culture cells was extracted by cell lysis. Recombinant protein was purified by affinity columns.

Protein Purification after Cloning and Expression

Total cell lysat of IPTG-induced bacteria were mixed with Ni-NTA matrix (Qiagen®) for 60 min et 4° C. and loaded into a column. After washing the column to remove non specific binding, the recombinant protein was eluate 3 times with 1 ml elution buffer pH 5.9. The protein was then eluate 4 times with 1 ml elution buffer pH 4.5.

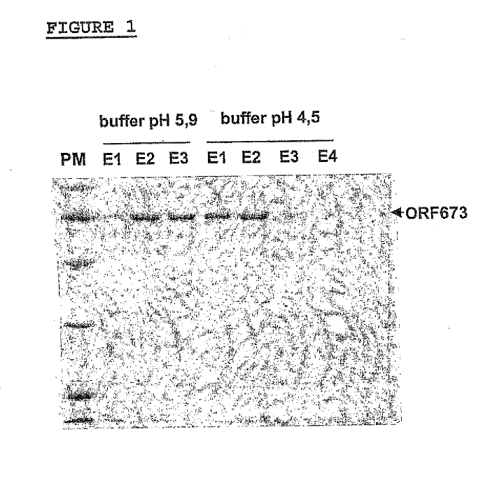

FIG. 1 represents a Coomassie blue stained SDS gel of recombinant protein after affinity column purification: PM: markers E1-4: sample collected from each purification fraction. Arrow indicate the band corresponding to the recombinant protein.

Test for Immunogenicity in an Animal Model

Polypeptide preparation from SEQ ID NO:28 was injected to Swiss mice to induce an antibody response as follows:

At d0 a first immunisation was done by injecting 20 μg of the protein at in 100 μg solution of PBS and complet Freund adjuvant (1:1). Control animals were injected with 100 μl solution of PBS and complet Freund adjuvant (1:1).

Boosting injection at d21 with 10 μg of protein in 100 μl PBS and complet Freund adjuvant (1:1).

Sera from vaccinated animals was prepared from blood drawn by puncture in the tail of the mice.

Detection of specific antibodies in animal sera, at d20 before the boosting injection, was performed by western blot according to standard protocol. Purified polypeptide was subjected to electrophoresis (10 μg per lane) and transferred to nitrocellulose membrane.

The membranes were then saturated by incubation 35 min with PBS/TWEEN20 (polyoxyethylene derivative of sorbitan monolaurate, and is distinguished from the other members in the Tween range by the length of the polyoxyethylene chain) 0.1%/powder milk 5%.

Diluted sera was incubated with the membrane for 45 min. Membranes were washed three time 5 min with PBS/tween. Bound antibodies were then recognized by an anti-mouse IgG coupled to horseradish peroxidase enzyme. After washing 3 times with PBS/Tween and 3 time with PBS, enzymatic activity was revealed by addition of chromogenic substrate DAB and hydrogen peroxyde.

Results: Sera from vaccinated animal, diluted at 1/100 revealed a unique band corresponding to the injected polypeptide. No antibody to the polypeptide could be detected in sera from control animals.

At d42, 300 μl of cyclophosphamide and 200 μl at d45 were injected IP in the mice to induce neutropenia in order to increase the susceptibility to the challenge infection.

At d46 vaccinated and control mice were challenged by intraperitoneal injection of the wt B2/D strain C5 of E. coli at a dose equal to 10 time the LD50 (lethal dose).

Immunogenicity of the selected polypeptide and protection conferred by vaccination with the selected polypeptide was assessed by the survival of vaccinated animals three days post challenge.

Another example of vaccination to demonstrate immunogenicity of polypeptides:
24 Balb/c mice, female, 6 weeks old were immunized by subcutaneous Injection of the protein in Complete Freund's adjuvant, and 14 control mice were injected with CFA and PBS
3 weeks later a boost injection of 10 μg of protein in with incomplete Freund's adjuvant.

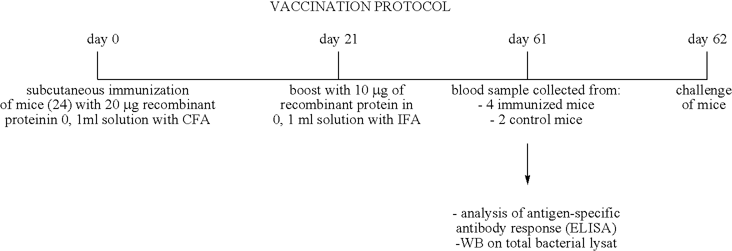

Before challenge at d62, sera was collected at d61 to analyze the antibody response in the vaccinated animals.
WB analysis of sera from immunized mice were performed to detect the antibody response to the recombinant protein used for immunization as described above.
An ELISA assay was set up to measure antibody titer in vaccinated animals:
Each recombinant protein was coated on a 96-well plates with (200 ng/100 μl coating buffer), plates are saturated with 3% BSA in PBS.
Twofold serial dilution of sera were made in PBS 1×/1% BSA from 1:500, to 1:1024000 and incubated on the plate, antibody binding was revealed using a rabbit Anti-mouse IgG conjugated with Peroxydase and OPD (o-phenylenediamine) chromogen substrate.
Read the OD₄₉₅ with Sanofi Diagnostics Pasteur PR2100®

Results:

TABLE 1
Sera titer against recombinant protein by ELISA

| SEQ | | | | | | recombinant |
|---|---|---|---|---|---|---|
| ID NOs: | serum1 | serum 2 | serum 3 | serum 4 | Control | protein |
| 2 | 128 | 64 | 128 | 512 | <0.5 | |
| 140 | >64 | >64 | >64 | >64 | <0.5 | + |
| 31 | >64 | >64 | >64 | >64 | <0.5 | + |
| 49 | >64 | >64 | >64 | >64 | <0.5 | + |
| 51 | >64 | >64 | >64 | >64 | <0.5 | + |
| 25 | >64 | >64 | >64 | >64 | <0.5 | + |
| 7 | 16 | 8 | >64 | <0.5 | <0.5 | + |
| 19 | >64 | >64 | >64 | >64 | 0.5 | + |
| 3 | >64 | >64 | >64 | >64 | <0.5 | + |
| 26 | 512 | 128 | 64 | 256 | <0.5 | + |
| 18 | >64 | >64 | >64 | >64 | 0.5 | + |
| 32 | >64 | >64 | >64 | >64 | 0.5 | + |
| 53 | >64 | >64 | >64 | >64 | 0.5 | + |
| 587 | >64 | 16 | 32 | 32 | <0.5 | + |
| 11 | >64 | 32 | 32 | 64 | <0.5 | + |
| 36 | 512 | 256 | 512 | 256 | <0.5 | + |
| 10 | 32 | 128 | 256 | 128 | 0.5 | + |
| 47 | 512 | 512 | 512 | 512 | 0.5 | + |
| 20 | 1024 | 256 | 256 | 512 | <0.5 | + |
| 17 | 1024 | 512 | 128 | 512 | <0.5 | + |

To assess the ability of sera to recognize the native antigen expressed by the bacteria, western blot was also performed on whole bacteria lysat.

To this end, bacteria were grown in LB medium supplemented or not with iron chelator until OD600.0.5-0.6 and pelleted by centrifugation 5 minutes at 10000 rpm. The pellet was lysed by resuspension in 1× loading buffer containing SDS and heated 5 min at 95° C. before migration on the gel. Western blot assay was then performed with sera from controls and vaccinated animals.

Results in table 2 shows the results obtained with Sera from vaccinated mice against recombinant protein and against E. coli lysat.

TABLE 2
reactivity in Western Blot of sera from mice vaccinated
with polypeptides encoded by the different ORFs

| SEQ ID | whole cell | recombinant |
|---|---|---|
| NO: | lysate | protein |
| 2 | + | + |
| 140 | + | + |
| 26 | + | + |

Protection assay, end point: mortality

At d62, 20 vaccinated and 10 control mice were challenged with an E. coli virulent strain belonging to B2 group at a dose equal to the LD 50 (5.10⁵ cfu/mice) by intraperitoneal injection. Mortality is recorded at 48 h, results in Table 3 are expressed as a percentage of protection representing the difference of survival in vaccinated versus control mice groups.

TABLE 3
Protection obtained in mice challenged after immunization
with proteines encoded by the corresponding ORFs.

| SEQ ID NO: | % protection |
|---|---|
| 2 | 52 |
| 26 | 66 |
| 36 | 46 |
| 10 | 30 |
| 47 | 60 |
| 20 | 25 |

Protection assay, end point: bacteremia

At d62, 10 vaccinated and 5 control mice were challenged with an Ecoli virulent strain belonging to B2 group at: a dose equal to the 1/5 of the LD 50 (1.10⁵ cfu/mice) by intraperitoneal injection. With this infectious dose the mice survived the infection at d48. At 48 h blood was collected for each mice in presence of heparin. To assess bacteremia, the blood was plated on LB media and colonies count measured after overnight culture.

### EXAMPLE 2

### Distribution of the DNA Sequence of ORFs Specific for B2/D Group of E. Coli in Clinical Isolates

To make a DNA arrays membrane specific for B2/D group of E. coli, DNA corresponding to ORFs that were identified as specific for B2/D core genome of E. coli was amplified by PCR and spotted on nylon membrane using standard methods to those skilled in the art.

Chromosomal DNA from 30 E. coli clinical isolate strains (of which 23 were from pathological conditions and 6 isolated from human normal flora), was prepared and radiolabelled with 33P.

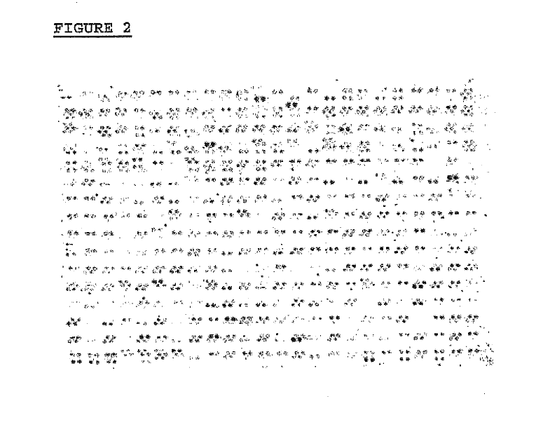

DNA from these clinical isolates was then hybridized to the B2/D specific DNA array, the results were read by a phosphoimager and spots reactivity was analyzed with an image analysis software. If hybridization gave a positive signal for a particular ORF, this ORf is considered to be present in the genome of the isolate. Quality control of the array is the hybridization of a probe DNA from a reference strain of E. coli as shown in FIG. 2, which represents a picture of the DNA array after hybridization with the genomic DNA from a B2/D reference strain.

The details of the method used for these experiments has been described previously in Tinsley et al. Methods Enzymol. 2002, 358; 188-207.

Results presented in Table 4 are expressed as the frequency of each ORF detection in the three different group A, B and D of E. coli strains.

TABLE 4
Presence of Orfs encoding antigens in
E. coli clinical isolate genomes

| | Frequency of positive signal % | | |
|---|---|---|---|
| | Clinical isolate groups | | |
| | A | D | B2 |
| SEQ ID NO: | N = 6 | n = 5 | n = 18 |
| 86 | 17 | 20 | 100 |
| 119 | 0 | 80 | 100 |
| 137 | 0 | 0 | 100 |
| 77 | 0 | 100 | 94 |
| 78 | 0 | 100 | 100 |
| 79 | 0 | 100 | 100 |
| 80 | 0 | 0 | 100 |
| 84 | 0 | 0 | 100 |
| 82 | 0 | 0 | 100 |
| 88 | 0 | 0 | 100 |
| 83 | 0 | 0 | 94 |
| 85 | 0 | 0 | 78 |
| 88 | 0 | 0 | 56 |
| 81 | 33 | 20 | 100 |
| 89 | 33 | 60 | 67 |
| 90 | 0 | 0 | 61 |
| 91 | 17 | 80 | 67 |
| 94 | 0 | 0 | 100 |
| 92 | 0 | 0 | 100 |
| 93 | 0 | 0 | 100 |
| 96 | 0 | 100 | 100 |
| 85 | 17 | 100 | 100 |
| 97 | 0 | 70 | 100 |
| 98 | 0 | 0 | 100 |
| 99 | 0 | 0 | 78 |
| 101 | 0 | 80 | 100 |
| 102 | 0 | 0 | 100 |
| 104 | 0 | 0 | 11 |
| 103 | 0 | 0 | 100 |
| 105 | 0 | 0 | 67 |
| 100 | 0 | 100 | 100 |
| 111 | 17 | 0 | 61 |
| 115 | 0 | 0 | 78 |
| 114 | 0 | 0 | 83 |
| 113 | 0 | 0 | 94 |
| 120 | 33 | 80 | 16 |
| 125 | 17 | 0 | 89 |
| 109 | 0 | 0 | 33 |
| 110 | 0 | 0 | 17 |
| 124 | 0 | 0 | 72 |
| 126 | 0 | 80 | 78 |
| 116 | 0 | 0 | 33 |
| 112 | 0 | 0 | 22 |
| 106 | 17 | 0 | 33 |
| 117 | 33 | 80 | 11 |
| 132 | 33 | 80 | 22 |
| 122 | 0 | 0 | 22 |
| 74 | 0 | 0 | 89 |
| 70 | 0 | 0 | 33 |
| 73 | 0 | 0 | 89 |
| 71 | 50 | 100 | 22 |
| 75 | 0 | 0 | 0 |
| 76 | 0 | 0 | 56 |
| 67 | 0 | 0 | 50 |
| 69 | 100 | 100 | 100 |
| 68 | 67 | 100 | 100 |
| 152 | 0 | 0 | 94 |
| 153 | 0 | 100 | 100 |
| 150 | 0 | 0 | 11 |
| 142 | 83 | 100 | 78 |
| 157 | 67 | 0 | 94 |
| 156 | 17 | 100 | 100 |
| 100 | 0 | 100 | 100 |
| 154 | 0 | 80 | 67 |
| 147 | 0 | 0 | 100 |
| 146 | 17 | 100 | 100 |
| 158 | 17 | 100 | 89 |
| 107 | 17 | 0 | 78 |
| 72 | 50 | 100 | 44 |
| 151 | 0 | 0 | 11 |
| 149 | 0 | 0 | 28 |
| 148 | 0 | 0 | 6 |

### EXAMPLE 3

### Vaccines Compositions Intended for Prevention of Any Form of Infection by ExPEC

The polypeptide coded by a sequence comprising SEQ ID NO:28 is conjugated with a toxin and added to a physiologically inert vehicle.

This conjugated peptide is optionally added to a childhood vaccine.

The composition is sterilized and can be injected parenterally, subcutaneously or intramuscularly.

Said composition can also be sprayed onto mucosa with the aid of a spray.

1. An isolated antigenic polypeptide obtainable by a process comprising the steps of:
a—selecting on the basis of sequence analysis polypeptides which are either located in the outermembrane or secreted by a bacteria,
b—identifying the genes coding for said polypeptides which are conserved in B2/D E. coli. clinical isolates,
c—preparing the polypeptides selected in step a, which are found in step b to be conserved in B2/D E. coli. isolates,
d—testing the polypeptides for immunogenicity using an animal model and isolating polypeptides which are antigenic.

2. An isolated polypeptide which is an antigenic fragment of SEQ ID NO:134, provided that said polypeptide is not SEQ ID NO:8.

3. A vaccine composition specific to E. coli extra-intestinal infections, comprising an effective amount of at least one polypeptide having the sequence set forth in SEQ ID NO:134 or a sequence which is an antigenic fragment of SEQ ID NO:134, provided that said antigenic fragment is not a peptide consisting of a sequence set forth in SEQ ID NO:8, said composition further comprising a pharmaceutically acceptable carrier.

4. The vaccine composition of claim 3 in a form administrable for treatment of a urinary system infection, pyelonephritis, sepsis, bacteremia, or neonatal meningitis.

5. The vaccine composition of claim 3, further comprising at least one component of S. aureus or a group B Streptococcus.

6. A method for detecting the presence or absence of undesirable extra-intestinal E. coli, and/or for the diagnosis of an extra-intestinal E. coli infection, comprising the use of the polypeptide having a sequence set forth in SEQ ID NO:134 or a polypeptide defined in claim 2, said polypeptide being optionally in combination with at least one polypeptide having a sequence selected from the group consisting of SEQ ID NOs:1-66 and 133-145.

7. Pharmaceutical composition for alleviating and/or preventing and/or treating an undesirable growth of E. coli comprising an effective amount of at least one polypeptide having a sequence set forth in SEQ ID NO:134 or a polypeptide according to claim 2, in combination with a pharmaceutically acceptable carrier.

8. A composition comprising a polypeptide having a sequence set forth in SEQ ID NO:134 or a polypeptide of claim 2, provided that said polypeptide does not consist of a sequence set forth in SEQ ID NO:8, with a pharmaceutically acceptable carrier.

9. The composition of claim 8 wherein said polypeptide has a sequence set forth in SEQ ID NO:134.

10. A method of making a composition of claim 8 comprising combining said polypeptide and said carrier.

11. The method of claim 10 wherein said polypeptide has the sequence set forth in SEQ ID NO:134.

12. A human vaccine specific to B2/D E. coli extra-intestinal infections, comprising an effective amount of a polypeptide having the sequence set forth in SEQ ID NO:134, and a pharmaceutically acceptable carrier.

13. The vaccine composition of claim 12 in a form administrable for treatment of a urinary system infection, pyelonephritis, sepsis, bacteremia, or neonatal meningitis.

14. The vaccine composition of claim 12, further comprising at least one component of S. aureus or a group B Streptococcus.

15. A pharmaceutical composition for treating an undesirable growth of B2/D E. coli comprising an effective amount of a polypeptide having a sequence set forth in SEQ ID NO:134, in combination with a pharmaceutically acceptable carrier.

16. A human vaccine specific to B2/D E. coli extra-intestinal infections, comprising an effective amount of a polypeptide having the sequence of an antigenic fragment of the sequence set forth in SEQ ID NO:134, and a pharmaceutically acceptable carrier.

17. The vaccine composition of claim 16 in a form administrable for treatment of a urinary system infection, pyelonephritis, sepsis, bacteremia, or neonatal meningitis.

18. The vaccine composition of claim 16, further comprising at least one component of S. aureus or a group B Streptococcus.

19. A pharmaceutical composition for treating an undesirable growth of B2/D E. coli comprising an effective amount of a polypeptide having the sequence of an antigenic fragment of the sequence set forth in SEQ ID NO:134, in combination with a pharmaceutically acceptable carrier.
